(19) United States
(12) Patent Application Publication
(10) Pub. No.: US 2009/0048252 A1
(43) Pub. Date: Feb. 19, 2009

(54) THIAZOLONES FOR USE AS PI3 KINASE INHIBITORS

(75) Inventors: Dashyant Dhanak, Collegeville, PA (US); Steven David Knight, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/281,181

(22) PCT Filed: Mar. 2, 2007
(86) PCT No.: PCT/US07/63113

§ 371 Date: Aug. 29, 2008

Related U.S. Application Data
(60) Provisional application No. 60/778,272, filed on Mar. 2, 2006.

(51) Int. Cl. A61K 31/427; A61K 31/5377; A61K 31/496; A61K 31/454

(52) U.S. Cl. 514/234.5; 514/369; 514/254.02; 514/322

(57) ABSTRACT

Invented is a method of inhibiting the activity/function of PI3 kinases using substituted thiazolones. Also invented is a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries by the administration of substituted thiazolones.

### FIELD OF THE INVENTION

This invention relates to the use of substituted thiazolones for the modulation, notably the inhibition of the activity or function of the phosphor-inositide-3′OH kinase family (hereinafter PI3 kinases), suitably, PI3Kα, PI3Kδ, PI3Kβ, and/or PI3Kγ. Suitably, the present invention relates to the use of substituted thiazolones in the treatment of one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

### BACKGROUND OF THE INVENTION

Cellular plasma membranes can be viewed as a large store of second messenger that can be enlisted in a variety of signal transduction pathways. As regards function and regulation of effector enzymes in phospholipids signaling pathways, these enzymes generate second messengers from the membrane phospholipids pool (class I PI3 kinases (e.g. PI3 Kgamma)) are dual-specific kinase enzymes, means they display both: lipid kinase (phosphorylation of phosphor-inositides) as well as protein kinase activity, shown to be capable of phosphorylation of other protein as substrates, including auto-phosphorylation as intramolecular regulatory mechanism. These enzymes of phospholipids signaling are activated in response to a variety of extra-cellular signals such as growth factors, mitogens, integrins (cell-cell interactions) hormones, cytokines, viruses and neurotransmitters such as described in Scheme 1 hereinafter and also by intra-cellular cross regulation by other signaling molecules (cross-talk, where the original signal can activate some parallel pathways that in a second step transmit signals to PI3Ks by intra-cellular signaling events), such as small GTPases, kinases or phosphatases for example. The inositol phospholipids (phosphoinositides) intracellular signaling pathway begins with binding of a signaling molecule (extra cellular ligands, stimuli, receptor dimerization, transactivation by heterologous receptor (e.g. receptor tyrosine kinase)) to a G-protein linked transmembrane receptor integrated into the plasma membrane.

PI3K converts the membrane phospholipids PIP(4,5)₂ into PIP(3,4,5)3 which in turn can be further converted into another 3′ phosphorylated form of phosphoinositides by 5′-specific phosphor-inositide phophatases, thus PI3K enzymatic activity results either directly or indirectly in the generation of two 3′-phosphoinositide subtypes that function as 2^{nd} messengers in intra-cellular signal transduction (Trends Biochem. Sci. 22(7) p. 267-72 (1997) by Vanhaesebroeck et al.: Chem. Rev. 101(8) p. 2365-80 (2001) by Leslie et al (2001); Annu. Rev. Cell. Dev. Biol. 17 p, 615-75 (2001) by Katso et al. and Cell. Mol. Life. Sci. 59(5) p. 761-79 (2002) by Toker et al.). Multiple PI3K isoforms categorized by their catalytic subunits, their regulation by corresponding regulatory subunits, expression patterns and signaling-specific functions (p110α, β, and γ) perform this enzymatic reaction (Exp. Cell. Res. 25 (1) p. 239-54 (1999) by Vanhaesebroeck and Katso et al., 2001, above).

The evolutionary conserved isoforms p110α and β are ubiquitously express, which δ and γ are more specifically expressed in the haematopoietic cell system, smooth muscle cells, myocytes and endothelial cells (Trends Biochem. Sci. 22(7) p. 267-72 (1997) by Vanhaesebroeck et al.). Their expression might also be regulated in an inducible manner depending on the cellular, tissue type and stimuli as well as disease context.

To date, eight mammalian PI3Ks have been identified, divided into three main classes (I, II, and III) on the basis of sequence homology, structure, binding partners, mode of activation, and substrate preference in vitro. Class I PI3Ks can phosphorylate phosphatidylinositol (PI), phosphatidylinositol-4-phosphate, m and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI (Vanhaesebrokeck et al., 1997, above; Vanhaesebroeck et al., 1999, above and Leslie et al, 2001, above) G-protein coupled receptors mediated phosphoinositide 3′OH-kinase activation via small GTPases such as Gβγ and Ras, and consequently PI3K signaling plays a central role in establishing and coordinating cell polarity and dynamic organization of the cytoskeleton—which together provides the driving force of cells to move.

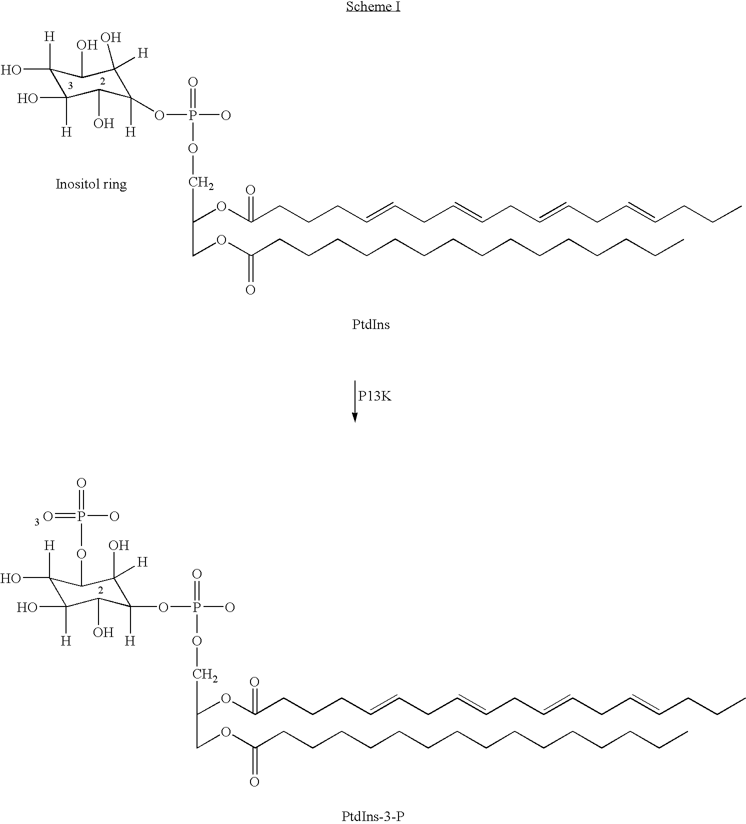

As illustrated in Scheme 1 above, Phosphoinositide 3-kinase (PI3K) is involved in the phosphorylation of Phosphatidylinositol (Ptdlns) on the third carbon of the inositol ring. The phosphorylation of Ptdlns to 3,4,5-triphosphate (Ptdlns(3,4,5)P₃), Ptdlns(3,4)P₂ and Ptdlns(3)P acts as second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Katso et al., 2001, above and Mol. Med. Today 6(9) p. 347-57 (2000) by Stein). Chemotaxis—the directed movement of cells toward a concentration gradient of chemical attractants, also called chemokines is involved in many important diseases such as inflammation/auto-immunity, neurodegeneration, antiogenesis, invasion/metastasis and wound healing (Immunol. Today 21(6) p. 260-4 (2000) by Wyman et al.; Science 287(5455) p. 1049-53 (2000) by Hirsch et al.; FASEB J. 15(11) p. 2019-21 (2001) by Hirsch et al. and Nat. Immunol. 2(2) p. 108-15 (2001) by Gerard et al.).

Recent advances using genetic approaches and pharmacological tools have provided insights into signalling and molecular pathways that mediate chemotaxis in response to chemoattractant activated G-protein coupled receptors PI3-Kinase, responsible for generating these phosphorylated signalling products, was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol. 2 p. 358-60 (1992)). However, more recent biochemical studies revealed that, class I PI3 kinases (e.g. class IB isoform PI3Kγ) are dual-specific kinase enzymes, means they display both: lipid kinase (phosphorylation of phospho-inositides) as well as protein kinase activity, shown to be capable of phosphorylation of other protein as substrates, including auto-phosphorylation as intra-molecular regulatory mechanism.

PI3-kinase activation, is therefore believe to be involved in a range of cellular responses including cell growth, differentiation, and apoptosis (Parker et al., Current Biology, 5 p. 577-99 (1995); Yao et al., Science, 267 p. 2003-05 (1995)). PI3-kinase appears to be involved in a number of aspects of leukocyte activation. A p85-associated PI3-kinase activity has been shown to physically associate with the cytoplasmic domain of CD28, which is an important costimulatory molecule for the activation of T-cells in response to antigen (Pages et al., Nature, 369 p. 327-29 (1994); Rudd, Immunity 4 p. 527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science 251 p. 313-16 (1991)). Mutation of CD28 such that it can longer interact with PI3-kinase leads to a failure to initiate IL2 production, suggesting a critical role for PI3-kinase in T cell activation. PI3Kγ has been identified as a mediator of G beta-gamma-dependent regulation of JNK activity, and G beta-gamma are subunits of heterotrimeric G proteins (Lopez-Ilasaca et al., J. Biol. Chem. 273(5) p. 2505-8 (1998)). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

Recently, (Laffargue et al., Immunity 16(3) p. 441-51 (2002)) it has been described that PI3Kγ relays inflammatory signals through various G(i)-coupled receptors and its central to mast cell function, stimuli in context of leukocytes, immunology includes cytokines, chemokines, adenosines, antibodies, integrins, aggregation factors, growth factors, viruses or hormones for example (J. Cell. Sci. 114(Pt 16) p. 2903-10 (2001) by Lawlor et al.; Laffargue et al., 2002, above and Curr. Opinion Cell Biol. 14(2) p. 203-13 (2002) by Stephens et al.).

Specific inhibitors against individual members of a family of enzymes provide invaluable tools for deciphering functions of each enzyme. Two compounds, LY294002 and wortmannin (cf. hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al., Ann. Rev. Biochem., 67, p. 481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates subsequent cellular response to the extracellular factor. For example, neutrophils respond to the chemokine fMet-Leu-Phe (fMLP) by stimulating PI3K and synthesizing Ptdlns (3, 4, 5)P₃. This synthesis correlates with activation of the respirators burst involved in neutrophil destruction of invading microorganisms. Treatment of neutrophils with wortmannin prevents the fMLP-induced respiratory burst response (Thelen et al., Proc. Natl. Acad. Sci. USA, 91, p. 4960-64 (1994)). Indeed, these experiments with wortmannin, as well as other experimental evidence, shows that PI3K activity in cells of hematopoietic lineage, particularly neutrophils, monocytes, and other types of leukocytes, is involved in many of the non-memory immune response associated with acute and chronic inflammation.

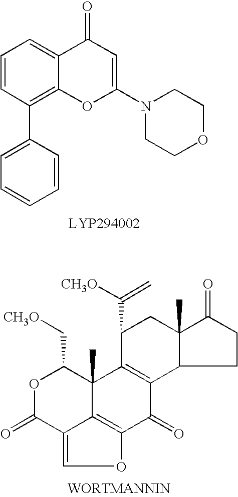

Based on studies using wortmannin, there is evidence that PI3-kinase function is also required for some aspects of leukocyte signaling through G-protein coupled receptors (Thelen et al., 1994, above). Moreover, it has been shown that wortmannin and LY294002 block neutrophil migration and superoxide release. Cyclooxygenase inhibiting benzofuran derivatives are disclosed by John M. Janusz et al., in J. Med. Chem. 1998; Vol. 41, No. 18.

It is now well understood that deregulation of onocogenes and tumour-suppressor genes contributes to the formation of malignant tumours, for example by way of increase cell proliferation or increased cell survival. It is also now known that signaling pathways mediated by the PI3k family have a central role in a number of cell processes including proliferation and survival, and deregulation of these pathways is a causative factor a wide spectrum of human cancers and other diseases (Katso et al., Annual Rev. Cell Dev. Biol. 2001, 17: 615-617 and Foster et al., J. Cell Science, 2003, 116: 3037-3040).

Class I PI3K is a heterodimer consisting of a p110 catalytic subunit and a regulatory subunit, and the family is further divided into class Ia and Class Ib enzymes on the basis of regulatory partners and mechanism of regulation. Class Ia enzymes consist of three distinct catalytic subunits (p110α, p110β, and p110δ) that dimerise with five distinct regulatory subunits (p85α, p55α, p50α, p85β, and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phosphor-tyrosine residues of the activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leukocytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh, et al., Nature Genetics, 1999, 21: 99-102) and cervix (Ma et al., Oncogene, 2000, 19: 2739-2744). More recently, activating mutations within p110α have been associated with various other tumors such as those of the colorectal region and of the breast and lung (Samuels, et al., Science, 2004, 304, 554). Tumor-related mutations in p85α have also been identified in cancers such as those of the ovary and colon (Philp et al., Cancer Research, 2001, 61, 7426-7429). In addition to direct effects, it is believed that activation of Class Ia PI3K contributes to tumourigenic events that occur upstream in signaling pathways, for example by way of ligan-dependent or ligand-independent activation of receptor tyrosine kinases, GPCR systems or integrins (Vara et al., Cancer Treatment Reviews, 2004, 30, 193-204). Examples of such upstream signaling pathways include over-expression of the receptor tyrosine kinase Erb2 in a variety of tumors leading to activation of PI3K-mediated pathways (Harari et al., Oncogene, 2000, 19, 6102-6114) and over-expression of the oncogene Ras (Kauffmann-Zeh et al., Nature, 1997, 385, 544-548). In addition, Class Ia PI3Ks may contribute indirectly to tumourigenesis caused by various downstream signaling events. For example, loss of the effect of the PTEN tumor-suppressor phosphatase that catalyses conversion of PI(3,4,5)P3 back to PI(4,5)P2 is associated with a very broad range of tumors via deregulation of PI3K-mediated production of PI(3,4,5)P3 (Simpson and Parsons, Exp. Cell Res., 2001, 264, 29-41). Furthermore, augmentation of the effects of other PI3K-mediated signaling events is believed to contribute to a variety of cancers, for example by activation of AKT (Nicholson and Andeson, Cellular Signaling, 2002, 14, 381-395).

In addition to a role in mediating proliferative and survival signaling in tumor cells, there is also good evidence that class Ia PI3K enzymes will also contribute to tumourigenesis via its function in tumor-associated stromal cells. For examples, PI3K signaling is known to play an important role in mediating angiogenic events in endothelial cells in response to pro-angiogenic factors such as VEGF (abid et al., Arterioscler, Thromb. Vasc. Biol., 2004, 24, 294-300). As Class I PI3K enzymes are also involved in motility and migration (Sawyer, Expert Opinion investing. Drugs, 2004, 13, 1-19), PI3K inhibitors should provide therapeutic benefit via inhibition of tumor cell invasion and metastasis.

### DESCRIPTION OF THE RELATED ART

International application No. PCT/US05/006022, filed Feb. 24, 2005, the entrie disclosure of which is hereby incorporated by reference, describes a group of thiazolidinone compounds which are indicated as having hYAK3 inhibitory activity and which are indicated as being useful in the treatment of deficiencies in hematopoietic cells, in particular in the treatment of deficiencies in erythroid cells.

International application No. PCT/US05/006022 does not disclose the use of any of the compounds described therein as inhibitors or inhibitors of PI3 kinases.

### SUMMARY OF THE INVENTION

This invention relates to a method of inhibiting one or more PI3 kinases selected from: PI3Kα, PI3Kδ, PI3Kβ and PI3Kγ, in a mammal in need thereof, which method comprises administrating to such mammal a therapeutically effective amount of a compound of Formula (I):

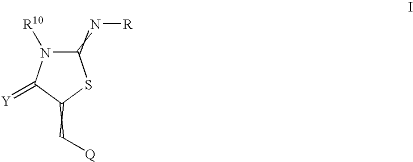

wherein:
R is selected from: hydrogen, aryl, substituted aryl, cycloalkyl, substituted cycloalkyl, C₁₋₆alkyl and substituted C₁₋₆alkyl;
R¹⁰ is selected from: hydrogen, C₁₋₆alkyl, —(CH₂)ₘOH and —(CH₂)ₘCOOH, where m is 0 to 6;
Y is selected from: ═O, ═S and ═NR¹¹,
   where R¹¹ is selected from: hydrogen, C₁₋₆alkyl, —(CH₂)ₚOH and —(CH₂)ₚCOOH,
   where p is 0 to 6; and
Q is a radical or substituted radical of the formula,

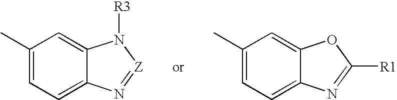

in which Z is N or C—R²;
wherein R² is hydrogen, —NH₂, —C₁₋₆alkyl, substituted —C₁₋₆alkyl, —CF₃, aryl or a radical or substituted radical of the formula

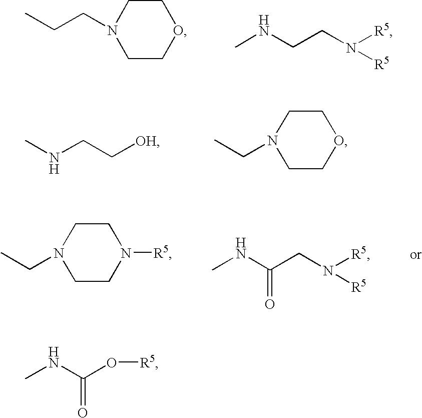

wherein R⁵ is selected from: hydrogen, —C₁₋₆alkyl and substituted —C₁₋₆alkyl; and
R³ is hydrogen, —C₁₋₆alkyl, substituted —C₁₋₆alkyl or C₃₋₁₂cycloalkyl; and
R¹ is hydrogen, —C₁₋₆alkyl, substituted —C₁₋₆alkyl, amino, mono substituted amino, disubstituted amino and trifluoromethyl,
   and/or a pharmaceutically acceptable salt, hydrate, solvate or pro-drug thereof.

This invention also relates to a method of treating cancer, which comprises administering to a subject in need thereof an effective amount of a compound of Formula (I).

This invention also relates to a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries, which comprises administering to a subject in need thereof an effective amount of a compound of Formula (I).

Included in the present invention are methods of co-administering the present PI3 kinase inhibiting compounds with further active ingredients.

### DETAILED DESCRIPTION OF THE INVENTION

The present invention relates to compounds of Formula (I) described above as PI3 kinase inhibitors. Suitably, the compounds of Formula (I) inhibit one or more PI3 kinases selected from: PI3Kα, PI3Kδ, PI3Kβ and PI3Kγ.

Included in the presently invented compounds of Formula I are those having Formula III, and/or pharmaceutically acceptable salts, hydrates, solvates, or pro-drugs thereof,

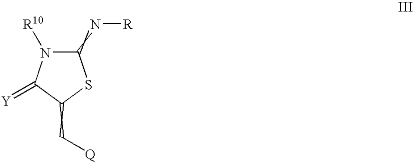

wherein:
R is selected from: hydrogen, C₁-C₁₂aryl, substituted C₁-C₁₂aryl, cycloalkyl, substituted cycloalkyl, C₁₋₆alkyl, —(CH₂)ₙ—NR^{k}R^{h}, —C(═NH)NH₂, —(CH₂)₂N(CH₃)₂, —C(═O)CH₃, —(CH₂)₂OCH₃, —(CH₂)₂OH, —(CH₂)₂C(CH₃)₃ and —(CH₂)CH(CH₃)₂, —C(═O)Ph, —C(═O)CH₂NHBOC, —(CH₂)₂CH(CH₃)₂,
where n is 0 to 6, and
R^{k} and R^{h} are independently selected form hydrogen, C₁₋₆alkyl and substituted C₁₋₆alkyl;
R¹⁰ is selected from: hydrogen, C₁₋₆alkyl, —(CH₂)ₘOH and —(CH₂)ₘCOOH, where m is 0 to 6;
Y is selected from: ═O, ═S and ═NR¹¹,
where R¹¹ is selected from: hydrogen, C₁₋₆alkyl, —(CH₂)ₚOH and —(CH₂)ₚCOOH,
where p is 0 to 6; and
Q is a radical of the formula,

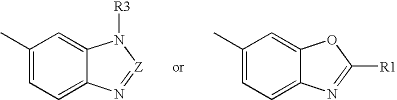

in which Z is N or C—R²;
wherein R² is hydrogen, —NH₂, —C₁₋₆alkyl, substituted —C₁₋₆alkyl, —CF₃, aryl or a radical of the formula

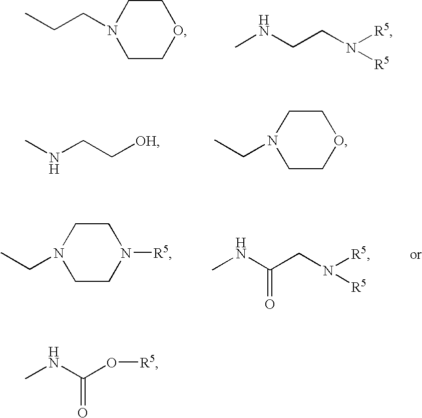

wherein R⁵ is selected from: hydrogen, —C₁₋₆alkyl and substituted —C₁₋₆alkyl; and R³ is hydrogen, —C₁₋₆alkyl, substituted —C₁₋₆alkyl or C₃₋₁₂cycloalkyl; and R¹ is hydrogen, —C₁₋₆alkyl, substituted —C₁₋₆alkyl, amino, mono substituted amino, disubstituted amino and trifluoromethyl.

Included in the presently invented compounds of Formula I are those having Formula IV, and/or pharmaceutically acceptable salts, hydrates, solvates, or pro-drugs thereof,

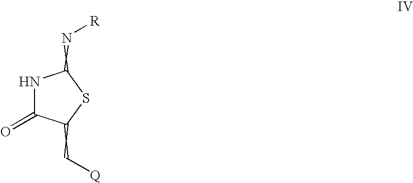

in which

### R is

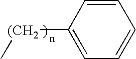

in which the phenyl radical is optionally and independently substituted with up to three substituents selected form: halogen, —C₁₋₆alkyl, —OC₁₋₆alkyl, —CF₃, —CN, —CO₂H, —SO₂NH₂, —CONH₂; or
R is a radical of the formula

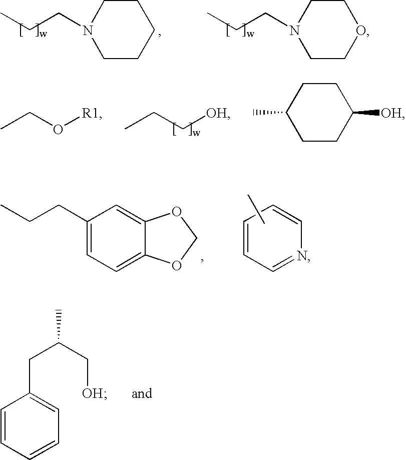

Q is a radical of the formula

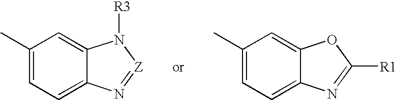

in which Z is N or C—R2;
wherein R2 is hydrogen, —NH₂, —C₁₋₆alkyl, —CF₃, or a radical of the formula

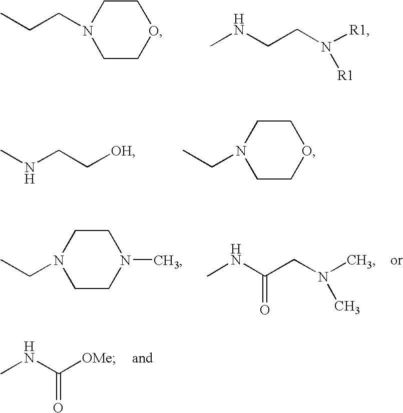

R3 is —C₁₋₆ alkyl, or a radical of the formula

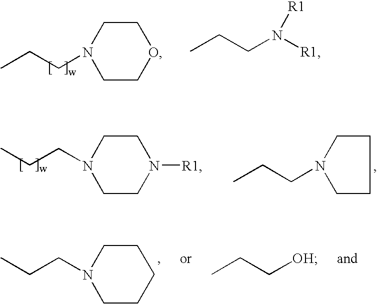

n equals zero to two;
w equals one to two; and
R1 is —C₁₋₆alkyl.
Included in the presently invented compounds of Formula IV are those in which R is phenyl optionally and independently substituted with up to three substituents selected form: halogen, —C₁₋₆alkyl, —OC₁₋₆alkyl, —CF₃, —CN, —CO₂H, —SO₂NH₂, —CONH₂.
Included in the presently invented compounds of Formula IV are those in which R is defined as a radical of the formula

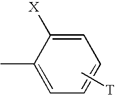

in which X is halogen or CF3; and T is selected from: hydrogen, halogen, —C₁₋₆alkyl, —OC₁₋₆alkyl, —CF₃, —CN, —CO₂H, —SO₂NH₂, —CONH₂.
Included in the presently invented compounds of Formula IV are those in which R is defined as a radical of the formula

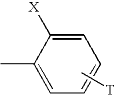

in which X is halogen or —CF3; and T is selected from: hydrogen, halogen, —C₁₋₆alkyl, —OC₁₋₆alkyl, —CF₃, —CN, —CO₂H, —SO₂NH₂, —CONH₂; and Q is

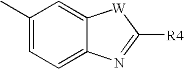

in which R⁴ is methyl or hydrogen, and W is O or N—R1, in which R1 is —C₁₋₆alkyl.
Included among the presently invented compounds are:
(5Z)-2-[(2-Chlorophenyl)amino]-5-[(1-methyl-1H-benzimidazol-6-yl)methylidene]-1,3-thiazol-4(5H)-one;
(5Z)-2-[(2,6-dichlorophenyl)amino]-5-[(1-methyl-1H-benzimidazol-6-yl)methylidene]-1,3-thiazol-4(5H)-one;
(5Z)-2-[(2,6-difluorophenyl)amino]-5-[(1-methyl-1H-benzimidazol-6-yl)methylidene]-1,3-thiazol-4(5H)-one;
(5Z)-2-[(2,4-dimethylphenyl)amino]-5-[(1-methyl-1H-benzimidazol-6-yl)methylidene]-1,3-thiazol-4(5H)-one;
(5Z)-5-[(1-methyl-1H-benzimidazol-6-yl)methylidene]-2-{[2-(methyloxy)phenyl]amino}-1,3-thiazol-4(5H)-one;
(5Z)-5-[(1-methyl-1H-benzimidazol-6-yl)methylidene]-2-{[2-(trifluoromethyl)phenyl]amino}-1,3-thiazol-4(5H)-one;
(5Z)-2-[(2,4-difluorophenyl)amino]-5-[(1-methyl-1H-benzimidazol-6-yl)methylidene]-1,3-thiazol-4(5H)-one;
(5Z)-2-[(2-chloro-4-fluorophenyl)amino]-5-[(1-methyl-1H-benzimidazol-6-yl)methylidene]-1,3-thiazol-4(5H)-one;
(5Z)-2-[(2-Chlorophenyl)-amino]-5-[(1,2-dimethyl-1H-benzimidazol-6-yl)-methylidene]-1,3-thiazol-4(5H)-one;
(5Z)-2-[(2,6-dichlorophenyl)amino]-5-[(1,2-dimethyl-1H-benzimidazol-6-yl)methylidene]-1,3-thiazol-4(5H)-one;
(5Z)-2-[(2,6-difluorophenyl)amino]-5-[(1,2-dimethyl-1H-benzimidazol-6-yl)methylidene]-1,3-thiazol-4(5H)-one;
(5Z)-5-[(1,2-dimethyl-1H-benzimidazol-6-yl)methylidene]-2-[(2,4-dimethylphenyl)amino]-1,3-thiazol-4(5H)-one;
(5Z)-2-[(2,4-difluorophenyl)amino]-5-[(1,2-dimethyl-1H-benzimidazol-6-yl)methylidene]-1,3-thiazol-4(5H)-one;
(5Z)-2-[(2-chloro-4-fluorophenyl)amino]-5-[(1,2-dimethyl-1H-benzimidazol-6-yl)methylidene]-1,3-thiazol-4(5H)-one;
(5Z)-2-[(2-Chlorophenyl)-amino]-5-({1-[2-(4-morpholinyl)ethyl]-1H-benzimidazol-6-yl}-methylidene)-1,3-thiazol-4(5H)-one;
(5Z)-2-[(2,6-dichlorophenyl)amino]-5-({1-[2-(4-morpholinyl)ethyl]-1H-benzimidazol-6-yl}methylidene)-1,3-thiazol-4(5H)-one;
(5Z)-2-[(2-chloro-4-fluorophenyl)amino]-5-({1-[2-(4-morpholinyl)ethyl]-1H-benzimidazol-6-yl}methylidene)-1,3-thiazol-4(5H)-one;
(5Z)-2-[(2-chlorophenyl)amino]-5-({1-[2-(dimethylamino)ethyl]-1H-benzimidazol-6-yl}methylidene)-1,3-thiazol-4(5H)-one;
(5Z)-2-[(2,6-dichlorophenyl)amino]-5-({1-[2-(dimethylamino)ethyl]-1H-benzimidazol-6-yl}methylidene)-1,3-thiazol-4(5H)-one;
(5Z)-2-[(2,4-difluorophenyl)amino]-5-({1-[2-(dimethylamino)ethyl]-1H-benzimidazol-6-yl}methylidene)-1,3-thiazol-4(5H)-one;
(5Z)-5-({1-[2-(dimethylamino)ethyl]-1H-benzimidazol-6-yl}methylidene)-2-(phenylamino)-1,3-thiazol-4(5H)-one;
(5Z)-2-[(2-chlorophenyl)amino]-5-({1-[2-(diethylamino)ethyl]-1H-benzimidazol-6-yl}methylidene)-1,3-thiazol-4(5H)-one;
(5Z)-2-[(2,6-dichlorophenyl)amino]-5-({1-[2-(diethylamino)ethyl]-1H-benzimidazol-6-yl}methylidene)-1,3-thiazol-4(5H)-one;
(5Z)-2-[(2-chlorophenyl)amino]-5-({1-[3-(4-morpholinyl)propyl]-1H-benzimidazol-6-yl}methylidene)-1,3-thiazol-4(5H)-one;
(5Z)-2-[(2,6-dichlorophenyl)amino]-5-({1-[3-(4-morpholinyl)propyl]-1H-benzimidazol-6-yl}methylidene)-1,3-thiazol-4(5H)-one;
(5Z)-2-[(2-chlorophenyl)amino]-5-({1-[3-(4-methyl-1-piperazinyl)propyl]-1H-benzimidazol-6-yl}methylidene)-1,3-thiazol-4(5H)-one;
(5Z)-2-[(2,6-dichlorophenyl)amino]-5-({1-[3-(4-methyl-1-piperazinyl)propyl]-1H-benzimidazol-6-yl}methylidene)-1,3-thiazol-4(5H)-one;
(5Z)-2-[(2-chlorophenyl)amino]-5-({1-[2-(1-pyrrolidinyl)ethyl]-1H-benzimidazol-6-yl}methylidene)-1,3-thiazol-4(5H)-one;
(5Z)-2-[(2,6-dichlorophenyl)amino]-5-({1-[2-(1-pyrrolidinyl)ethyl]-1H-benzimidazol-6-yl}methylidene)-1,3-thiazol-4(5H)-one;
(5Z)-2-[(2-chlorophenyl)amino]-5-({1-[2-(1-piperidinyl)ethyl]-1H-benzimidazol-6-yl}methylidene)-1,3-thiazol-4(5H)-one;
(5Z)-2-[(2,6-dichlorophenyl)amino]-5-({1-[2-(1-piperidinyl)ethyl]-1H-benzimidazol-6-yl}methylidene)-1,3-thiazol-4(5H)-one;
(5Z)-2-[(2,6-difluorophenyl)amino]-5-({1-[2-(1-piperidinyl)ethyl]-1H-benzimidazol-6-yl}methylidene)-1,3-thiazol-4(5H)-one;
(5Z)-2-[(2-Chlorophenyl)amino]-5-({1-[2-(dimethylamino)ethyl]-2-methyl-1H-benzimidazol-6-yl}methylidene)-1,3-thiazol-4(5H)-one;
(5Z)-2-[(2,6-dichlorophenyl)amino]-5-({1-[2-(dimethylamino)ethyl]-2-methyl-1H-benzimidazol-6-yl}methylidene)-1,3-thiazol-4(5H)-one;
(5Z)-2-[(2,4-difluorophenyl)amino]-5-({1-[2-(dimethylamino)ethyl]-2-methyl-1H-benzimidazol-6-yl}methylidene)-1,3-thiazol-4(5H)-one;
(5Z)-5-({1-[2-(dimethylamino)ethyl]-2-methyl-1H-benzimidazol-6-yl}methylidene)-2-(phenylamino)-1,3-thiazol-4(5H)-one;
(5Z)-2-[(2,6-dichlorophenyl)amino]-5-{[1-(2-hydroxyethyl)-2-methyl-1H-benzimidazol-6-yl]methylidene}-1,3-thiazol-4(5H)-one;
(5Z)-2-[(2-chlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-dichlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-difluorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2-chlorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2,6-dichlorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2,4-difluorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2-chlorophenyl)amino]-5-[(2-{[2-(dimethylamino)ethyl]amino}-1-methyl-1H-benzimidazol-6-yl)methylidene]-1,3-thiazol-4(5H)-one;
(5Z)-2-[(2-chlorophenyl)amino]-5-({2-[(2-hydroxyethyl)amino]-1-methyl-1H-benzimidazol-6-yl}methylidene)-1,3-thiazol-4(5H)-one;
(5Z)-2-[(2-chlorophenyl)amino]-5-{[1-methyl-2-(4-morpholinylmethyl)-1H-benzimidazol-6-yl]methylidene}-1,3-thiazol-4(5H)-one;
(5Z)-2-[(2,6-dichlorophenyl)amino]-5-{[1-methyl-2-(4-morpholinylmethyl)-1H-benzimidazol-6-yl]methylidene}-1,3-thiazol-4(5H)-one;
(5Z)-2-[(2-chlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2,6-dichlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2-Chlorophenyl)-amino]-5-{[1-methyl-2-(trifluoromethyl)-1H-benzimidazol-6-yl]-methylidene}-1,3-thiazol-4(5H)-one;
(5Z)-2-[(2,6-dichlorophenyl)amino]-5-{[1-methyl-2-(trifluoromethyl)-1H-benzimidazol-6-yl]methylidene}-1,3-thiazol-4(5H)-one;
(5Z)-2-[(2,6-dichlorophenyl)amino]-5-{[1-[2-(dimethylamino)ethyl]-2-(trifluoromethyl)-1H-benzimidazol-6-yl]methylidene}-1,3-thiazol-4(5H)-one;
(5Z)-2-[(2-chlorophenyl)amino]-5-{[2-(1,1-dimethylethyl)-1-methyl-1H-benzimidazol-6-yl]methylidene}-1,3-thiazol-4(5H)-one;
(5Z)-2-[(2,6-dichlorophenyl)amino]-5-{[2-(1,1-dimethylethyl)-1-methyl-1H-benzimidazol-6-yl]methylidene}-1,3-thiazol-4(5H)-one;
(5Z)-2-[(2-Chlorophenyl)amino]-5-[(1-methyl-1H-1,2,3-benzotriazol-6-yl)methylidene]-1,3-thiazol-4(5H)-one;
(5Z)-2-[(2,6-dichlorophenyl)amino]-5-[(1-methyl-1H-1,2,3-benzotriazol-6-yl)methylidene]-1,3-thiazol-4(5H)-one;
(5Z)-2-[(2-chlorophenyl)amino]-5-({1-[2-(dimethylamino)ethyl]-1H-1,2,3-benzotriazol-6-yl}methylidene)-1,3-thiazol-4(5H)-one;
(5Z)-2-[(2,6-dichlorophenyl)amino]-5-({1-[2-(dimethylamino)ethyl]-1H-1,2,3-benzotriazol-6-yl}methylidene)-1,3-thiazol-4(5H)-one;
2-(2,6-Dichloro-phenylimino)-5-(2-methyl-benzooxazol-6-yl-methylene)-thiazolidin-4-one;
2-(2,6-Difluoro-phenylimino)-5-(2-methyl-benzooxazol-6-yl-methylene)-thiazolidin-4-one;
2-(2-Fluoro-phenylimino)-5-(2-methyl-benzooxazol-6-yl-methylene)-thiazolidin-4-one;
2-(2-Chloro-phenylimino)-5-(2-methyl-benzooxazol-6-ylmethylene)-thiazolidin-4-one;
2-(2-Trifluromethyl-phenylimino)-5-(2-methyl-benzooxazol-6-ylmethylene)-thiazolidin-4-one;
2-(2,4-Difluoro-phenylimino)-5-(2-methyl-benzooxazol-6-ylmethylene)-thiazolidin-4-one;
2-(2,5-Dichloro-phenylimino)-5-(2-methyl-benzooxazol-6-ylmethylene)-thiazolidin-4-one;
2-(2,4-Dimethyl-phenylimino)-5-(2-methyl-benzooxazol-6-ylmethylene)-thiazolidin-4-one;
2-(4-Cyano-phenylimino)-5-(2-methyl-benzooxazol-6-ylmethylene)-thiazolidin-4-one;
4-[5-(2-Methyl-benzooxazol-6-ylmethylene)-4-oxo-thiazolidin-2-ylideneamino]-benzoic acid;
2-(2,4-Dichloro-phenylimino)-5-(2-methyl-benzooxazol-6-ylmethylene)-thiazolidin-4-one;
2-(2,5-Difluoro-phenylimino)-5-(2-methyl-benzooxazol-6-ylmethylene)-thiazolidin-4-one;
5-(2-Methyl-benzooxazol-6-ylmethylene)-2-phenylimino-thiazolidin-4-one;
5-(2-Methyl-benzooxazol-6-ylmethylene)-2-(2-piperidin-1-yl-ethylimino)-thiazolidin-4-one;
2-(2-Methoxy-ethylimino)-5-(2-methyl-benzooxazol-6-ylmethylene)-thiazolidin-4-one;
5-(2-Methyl-benzooxazol-6-ylmethylene)-2-(3-morpholin-4-yl-propylimino)-thiazolidin-4-one;
3-[5-(2-Methyl-benzooxazol-6-ylmethylene)-4-oxo-thiazolidin-2-ylideneamino]-benzenesulfonamide;
2-(4-Hydroxy-butylimino)-5-(2-methyl-benzooxazol-6-ylmethylene)-thiazolidin-4-one;
2-(trans-4-Hydroxy-cyclohexylimino)-5-(2-methyl-benzooxazol-6-ylmethylene)-thiazolidin-4-one;
5-(2-Methyl-benzooxazol-6-ylmethylene)-2-phenethylimino-thiazolidin-4-one;
4-{2-[5-(2-Methyl-benzooxazol-6-ylmethylene)-4-oxo-thiazolidin-2-ylideneamino]-ethyl}-benzenesulfonamide;
2-(2-Benzo[1,3]dioxol-5-yl-ethylimino)-5-(2-methyl-benzooxazol-6-ylmethylene)-thiazolidin-4-one;
2-(4-Chloro-phenylimino)-5-(2-methyl-benzooxazol-6-ylmethylene)-thiazolidin-4-one;
5-(2-Methyl-benzooxazol-6-ylmethylene)-2-(pyridin-3-ylimino)-thiazolidin-4-one;
3-[5-(2-Methyl-benzooxazol-6-ylmethylene)-4-oxo-thiazolidin-2-ylideneamino]-benzamide;
2-(2-Hydroxy-ethylimino)-5-(2-methyl-benzooxazol-6-ylmethylene)-thiazolidin-4-one;
2-(1-Hydroxymethyl-2-phenyl-ethylimino)-5-(2-methyl-benzooxazol-6-ylmethylene)-thiazolidin-4-one;
N-{6-[2-(2-Bromo-phenylimino)-4-oxo-thiazolidin-5-ylidenemethyl]-1H-benzoimidazol-2-yl}-2-dimethylamino-acetamide;
Methyl (5-{(Z)-[2-[(2-bromophenyl)amino]-4-oxo-1,3-thiazol-5(4H)-ylidene]methyl}-1H-benzimidazol-2-yl)carbamate;
(5Z)-2-[(2-chlorophenyl)amino]-5-{[1-(3,3-dimethylbutyl)-1H-benzimidazol-6-yl]methylidene}-1,3-thiazol-4(5H)-one;
(5Z)-2-[(2,6-dichlorophenyl)amino]-5-{[1-(3,3-dimethyl butyl)-1H-benzimidazol-6-yl]methylidene}-1,3-thiazol-4(5H)-one, trifluoroacetate salt;
(5Z)-5-{[1-(2-cyclopropylethyl)-1H-benzimidazol-6-yl]methylidene}-2-[(2,6-difluorophenyl)amino]-1,3-thiazol-4(5H)-one;
(5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt;
(2Z,5Z)-2-[(2,6-dichlorophenyl)imino]-5-[(2-phenyl-1H-benzimidazol-6-yl)methylidene]-1,3-thiazolidin-4-one, piperidine salt;
(5Z)-2-[(2-chlorophenyl)amino]-5-{[1-(2-cyclopropylethyl)-1H-benzimidazol-6-yl]methylidene}-1,3-thiazol-4(5H)-one;
(5Z)-5-{[1-(2-cyclopropylethyl)-1H-benzimidazol-6-yl]methylidene}-2-[(2,6-dichlorophenyl)amino]-1,3-thiazol-4(5H)-one;
(2Z,5Z)-2-[(2,6-dichlorophenyl)imino]-5-{[2-(1-methylethyl)-1H-benzimidazol-6-yl]methylidene}-1,3-thiazolidin-4-one;
(2Z,5Z)-2-[(2,6-dichlorophenyl)imino]-5-{[2-(2-methylpropyl)-1H-benzimidazol-6-yl]methylidene}-1,3-thiazolidin-4-one, piperidine salt;
(2Z,5Z)-2-[(2,6-dichlorophenyl)imino]-5-{[2-(3-pyridinyl)-1H-benzimidazol-6-yl]methylidene}-1,3-thiazolidin-4 one;
(2Z,5Z)-2-[(2,6-dichlorophenyl)imino]-5-{[2-(hydroxymethyl)-1H-benzimidazol-5-yl]methylidene}-1,3-thiazolidin-4-one;
(2Z,5Z)-2-[(2,6-dichlorophenyl)imino]-5-{[2-(2-hydroxyethyl)-1H-benzimidazol-5-yl]methylidene}-1,3-thiazolidin-4-one;
(2Z,5Z)-2-[(2,6-dichlorophenyl)imino]-5-{[2-(2-pyridinyl)-1H-benzimidazol-6-yl]methylidene}-1,3-thiazolidin-4-one;
(5Z)-5-{[1-(2-cyclopentylethyl)-1H-benzimidazol-6-yl]methylidene}-2-[(2,6-dichlorophenyl)amino]-1,3-thiazol-4(5H)-one;
(5Z)-2-[(2-chlorophenyl)amino]-5-{[1-(2-cyclopentylethyl)-1H-benzimidazol-6-yl]methylidene}-1,3-thiazol-4(5H)-one;
(2Z,5Z)-2-[(2,6-dichlorophenyl)imino]-5-[(2-methyl-1H-benzimidazol-6-yl)methylidene]-1,3-thiazolidin-4-one;
(2Z,5Z)-2-[(2,6-dichlorophenyl)imino]-5-{[2-(4-pyridinyl)-1H-benzimidazol-5-yl]methylidene}-1,3-thiazolidin-4-one;
(2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one;
(2Z,5Z)-2-[(2,6-dichlorophenyl)imino]-5-{[1-methyl-2-(3-pyridinyl)-1H-benzimidazol-5-yl]methylidene}-1,3-thiazolidin-4-one;
(2Z,5Z)-5-{[2-(aminomethyl)-1H-benzimidazol-5-yl]methylidene}-2-[(2,6-dichlorophenyl)imino]-1,3-thiazolidin-4-one;
(2Z,5Z)-5-(1H-benzimidazol-5-ylmethylidene)-2-[(2,6-dichlorophenyl)imino]-1,3-thiazolidin-4-one;
(2Z,5Z)-2-[(2,6-dichlorophenyl)imino]-5-{[2-(hydroxymethyl)-1-methyl-1H-benzimidazol-6-yl]methylidene}-1,3-thiazolidin-4-one;
(5Z)-2-[(2,6-dichlorophenyl)amino]-5-({1-[2-(3-pyridinyl)ethyl]-1H-benzimidazol-6-yl}methylidene)-1,3-thiazol-4(5H)-one;
(5Z)-5-{[1-(cyclopropylmethyl)-1H-benzimidazol-6-yl]methylidene}-2-[(2,6-dichlorophenyl)amino]-1,3-thiazol-4(5H)-one;
(5Z)-5-[(1-cyclopentyl-1H-benzimidazol-6-yl)methylidene]-2-[(2,6-dichlorophenyl)amino]-1,3-thiazol-4(5H)-one; and
(5Z)-5-(1,3-Benzoxazol-6-ylmethylidene)-2-[(2,6-dichlorophenyl)amino]-1,3-thiazol-4(5H)-one;
   and/or a pharmaceutically acceptable salt, hydrate, solvate or pro-drug thereof.

The invention also relates to a pharmaceutical composition including a therapeutically effective amount of a compound of formula I, II, III, IV, or a salt, solvate, or a physiologically functional derivative thereof and one or more of pharmaceutically acceptable carriers, diluents and excipients.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiohpene, tetrahydrobenzothiohpene and tetrazole.

The term “substituted” as used herein, unless otherwise defined, is meant that the subject chemical moiety has one or more substituents selected from the group consisting of: aryl,

aryl substituted with one or more substituents selected from alkyl, hydroxy, alkoxy, oxo, C₁-C₁₂aryl optionally substituted with one or more substituents selected from hydroxy, alkoxy oxo, cyano, amino, alkylamino, dialkylamino, alkyl and alkoxy, trifluoromethyl, —SO₂NR²¹R²², N-acylamino, —CO₂R²⁰, and halogen,
cycloalkyl substituted with one or more substituents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more substituents selected from alkyl, hydroxy, alkoxy, —SO₂NR²¹R²², amino, —CO₂R²⁰, trifluoromethyl, N-acylamino and halogen,
alkoxy substituted with one or more substituents selected form alkyl, —CO₂H, hydroxyl, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²², —C(O)NR²¹R²², —S(O)₂NR²¹R²², —(CH₂)_{g}N(R²⁰)C(O)ₙR²⁰, —(CH₂)_{g}N═C(H)R²⁰, —C(O)R²⁰, acyloxy, —SC₁-C₆alkyl, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}C(O)OR²⁰, —(CH₂)_{g}S(O)ₙR²³, nitro, cyano, oxo, halogen, trifluoromethyloxy and trifluoromethyl;
where g is 0 to 6, n is 0 to 2, R²³ is hydrogen or alkyl, each R²⁰ is independently selected form hydrogen, alkyl, C₁-C₆alkyloxyC₁-C₆alkyl, C₁-C₄alkylC(O)OC₁-C₄alkyl, amino, alkylamino, dialkylamino, aminoC₁-C₆alkyl, alkylaminoC₁-C₆alkyl, dialkylaminoC₁-C₆alkyl, —C(O)OH, alkoxy, aryloxy, arylamino, diarylamino, arylalkylamino, aryl, aryl substituted with one or more substituents selected from oxo, hydroxyl and alkyl, arylC₁-C₄alkyl optionally substituted with one or more substituents selected from oxo, hydroxy, halogen, alkoxy and alkyl, —CH₂C(O)cycloalkyl containing from 1 to 4 heteroatoms, cycloalkylC₁-C₄alkyl, C₁-C₄alkyl substituted with cycloalkyl containing from 1 to 4 heteroatoms, cycloalkyl, cycloalkyl substituted with one or more substituents selected from oxo, hydroxyl and alkyl, cycloalkyl containing from 1 to 4 heteroatoms, cycloalkyl containing from 1 to 4 heteroatoms substituted with one or more substituents selected from oxo, hydroxyl and alkyl, and trifluoromethyl, and R²¹ and R²² are independently selected form hydrogen, alkyl, C₁-C₆alkyl substituted with one or more substituents selected from hydroxy, amino, ═NH, and ≡N, —S(O)₂aryl, —S(O)₂alkyl, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

By the term “naphthyridin-6-yl” as used herein, is meant 1,5-naphthyridin-6-yl, 1,7-naphthyridin-6-yl, and 1,8-naphthyridin-6-yl.

By the term “alkoxy” as used herein is meant -Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazine, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant -Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include:
—CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, —C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “optionally” means that the subsequently described event(s) may or may not occur, and includes both event(s), which occur, and events that do not occur.

As used herein, the crisscrossed double bond indicated by the symbol

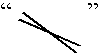

denotes Z and/or E stereochemistry around the double bond. In other words a compound of formula I or II can be either in the Z or E stereochemistry around this double bond, or a compound of formula I or II can also be in a mixture of Z and E stereochemistry around the double bond. However, in formulas I and II, the preferred compounds have Z stereochemistry around the double bond to which radical Q is attached.

The compounds of Formulas I and II naturally may exist in one tautomeric form or in a mixture of tautomeric forms. For example, for sake simplicity, compounds of formula I and II are expressed in one tautomeric form, usually as an exo form, i.e.

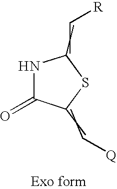

However, a person of ordinary skill can readily appreciate, the compounds of formulas I and II can also exist in endo forms.

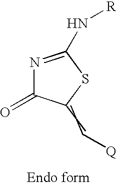

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I or II above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I or II.

Compounds of Formula (I) are included in the pharmaceutical compositions of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

It has now been found that compounds of the present invention are inhibitors of the Phosphatoinositides 3-kinases (PI3Ks). When the phosphatoinositides 3-kinase (PI3K) enzyme is inhibited by a compound of the present invention, PI3K is unable to exert its enzymatic, biological and/or pharmacological effects. The compounds of the present invention are therefore useful in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The compounds of Formula (I) are useful as medicaments in particular for the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries. According to one embodiment of the present invention, the compounds of Formula (I) are inhibitors of one or more phosphatoinositides 3-kinases (PI3Ks), suitably, Phosphatoinositides 3-kinase γ (PI3Kγ), Phosphatoinositides 3-kinase γ (PI3Kα), Phosphatoinositides 3-kinase γ (PI3Kβ), and/or Phosphatoinositides 3-kinase γ (PI3Kδ).

Compounds according to Formula (I) are suitable for the modulation, notably the inhibition of the activity of phosphatoinositides 3-kinases (PI3K), suitably phosphatoinositides 3-kinase (PI3Kγ). Therefore the compounds of the present invention are also useful for the treatment of disorders which are mediated by PI3Ks. Said treatment involves the modulation—notably the inhibition or the down regulation—of the phosphatoinositides 3-kinases.

Suitably, the compounds of the present invention are used for the preparation of a medicament for the treatment of a disorder selected from multiple sclerosis, psoriasis, rheumatoid arthritis, systemic lupus erythematosis, inflammatory bowel disease, lung inflammation, thrombosis or brain infection/inflammation, such as meningitis or encephalitis, Alzheimer's disease, Huntington's disease, CNS trauma, stroke or ischemic conditions, cardiovascular diseases such as athero-sclerosis, heart hypertrophy, cardiac myocyte dysfunction, elevated blood pressure or vasoconstriction.

Suitably, the compounds of Formula (I) are useful for the treatment of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

Suitably, the compounds of Formula (I) are useful for the treatment of neurodegenerative diseases including multiple sclerosis, Alzheimer's disease, Huntington's disease, CNS trauma, stroke or ischemic conditions.

Suitably, the compounds of Formula (I) are useful for the treatment of cardiovascular diseases such as atherosclerosis, heart hypertrophy, cardiac myocyte dysfunction, elevated blood pressure or vasoconstriction.

Suitably, the compounds of Formula (I) are useful for the treatment of chronic obstructive pulmonary disease, anaphylactic shock fibrosis, psoriasis, allergic diseases, asthma, stroke, ischemic conditions, ischemia-reperfusion, platelets aggregation/activation, skeletal muscle atrophy/hypertrophy, leukocyte recruitment in cancer tissue, angiogenesis, invasion metastasis, in particular melanoma, Karposi's sarcoma, acute and chronic bacterial and viral infections, sepsis, transplantation rejection, graft rejection, glomerulo sclerosis, glomerulo nephritis, progressive renal fibrosis, endothelial and epithelial injuries in the lung, and lung airway inflammation.

Because the pharmaceutically active compounds of the present invention are active as PI3 kinase inhibitors, particularly the compounds that inhibit PI3Kα, either selectively or in conjunction with one or more of PI3Kδ, PI3Kβ, and/or PI3Kγ, they exhibit therapeutic utility in treating cancer.

Suitably, the present invention relates to a method for treating or lessening the severity of a cancer selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, pancreatic, breast, prostate and leukemia.

When a compound of Formula (I) is administered for the treatment of cancer, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present PI3 kinase inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine[R—(R*, R*)-2,3-dihydroxybutanedioate(1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine[1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4-[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

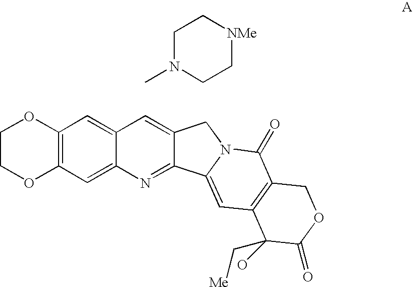

known by the chemical name “7-(4-methylpiperazino-methylene)-10,11-ethylenedioxy-20(R,S)-camptothecin (racemic mixture) or “7-(4-methyl piperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as PI3 kinase inhibitors, particularly the compounds that modulate/inhibit PI3Kγ, either selectively or in conjunction with one or more of PI3Kδ, PI3Kβ, and/or PI3Kα, they exhibit therapeutic utility in treating a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries.

When a compound of Formula (I) is administered for the treatment of a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection or lung injuries, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and/or lung injuries.

The pharmaceutically active compounds within the scope of this invention are useful as PI3 Kinase inhibitors in mammals, particularly humans, in need thereof.

The present invention therefore provides a method of treating diseases associated with PI3 kinase inhibition, particularly: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries and other conditions requiring PI3 kinase modulation/inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as PI3 inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a PI3K inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient. The above dosages relate to suitable amount of compound expressed as the free acid.

Optimal dosages to be administered may be readily determined by those skilled in the art, and will vary with the particular PI3 kinase inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing PI3 kinase inhibitory activity in mammals, including humans, comprises administering to a subject in need of such activity an effective PI3 kinase modulating/inhibiting amount of a pharmaceutically active compound of the present invention.

The invention also provides for the use of a compound of Formula (I) in the manufacture of a medicament for use as a PI3 kinase inhibitor.

The invention also provides for the use of a compound of Formula (I) in the manufacture of a medicament for use in therapy.

The invention also provides for the use of a compound of Formula (I) in the manufacture of a medicament for use in treating autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The invention also provides for a pharmaceutical composition for use as a PI3 inhibitor which comprises a compound of Formula (I) and a pharmaceutically acceptable carrier.

The invention also provides for a pharmaceutical composition for use in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, including compounds known to have utility when used in combination with a PI3 kinase inhibitor.

Without further elaboration, it is believed that one skilled in the art can, using the preceding description, utilize the present invention to its fullest extent. The following examples are, therefore, to be construed as merely illustrative and not a limitation of the scope of the present invention in any way.

For ease of illustration, the regiochemistry around the double bonds in the chemical formulas in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of either Scheme A or B or a variant thereof. Any person skilled in the art can readily adapt the process of either A or B, such the stoichemistry of the reagents, temperature, solvents, etc. to optimize the yield of the products desired.

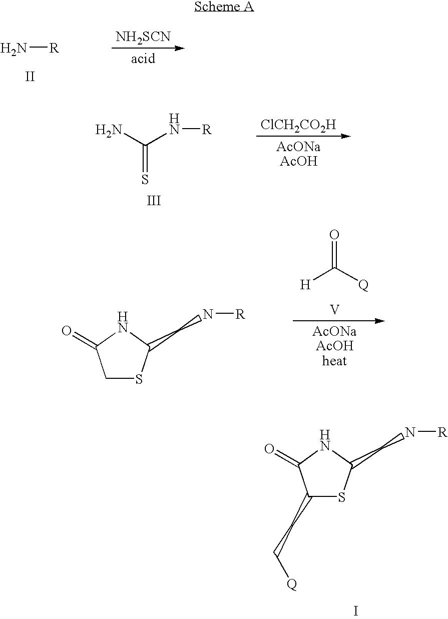

Briefly in Scheme A, a mixture of aniline derivative of formula II (1 equivalent) and NH4SCN (about 1.3 equivalent) in an acid (typically 4N—HCl) is heated to reflux at about 110° C. for 6 hours. After cooling, the mixture is treated with H₂O, which process usually forms a solid, followed by desiccation in vacuo to give a compound of formula III. (However, the compounds of formula III are often commercially available.)

A mixture of formula III compound, ClCH₂CO₂H (1 equivalent), and AcONa (1 equivalent) in AcOH is heated to reflux at around 110° C. for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), AcONa (3 equivalent) in AcOH is heated to reflux at about 110° C. for about 10 to 48 hours. After cooling, a small portion of water was added until the solid forms. The solid is filtered and washed with a solvent such as MeOH, followed by desiccation in vacuo to afford a target product of formula I.

As a variation of Scheme A, a compound of formula IV can also be synthesized according to Scheme A′ or Scheme A″.

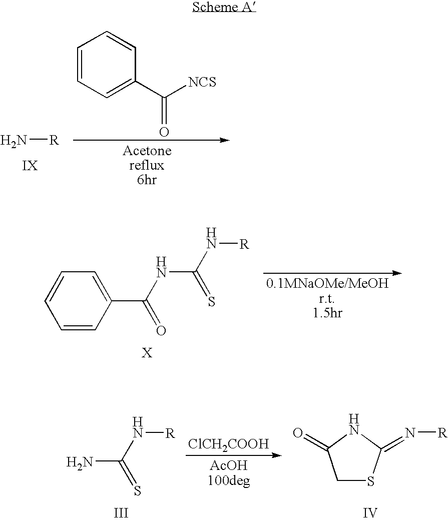

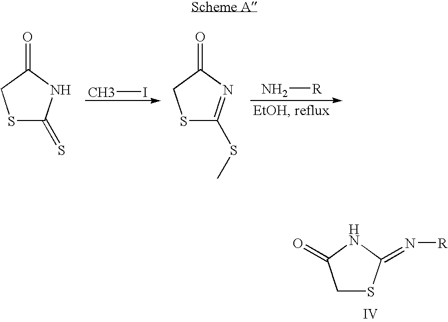

Compounds of formula V are known or can be made by standard organic chemical techniques. For example, Schemes 1 to 11 depict some of the ways to make a compound of formula V, and further ways to make a compound of formula I from a compound of formula V.

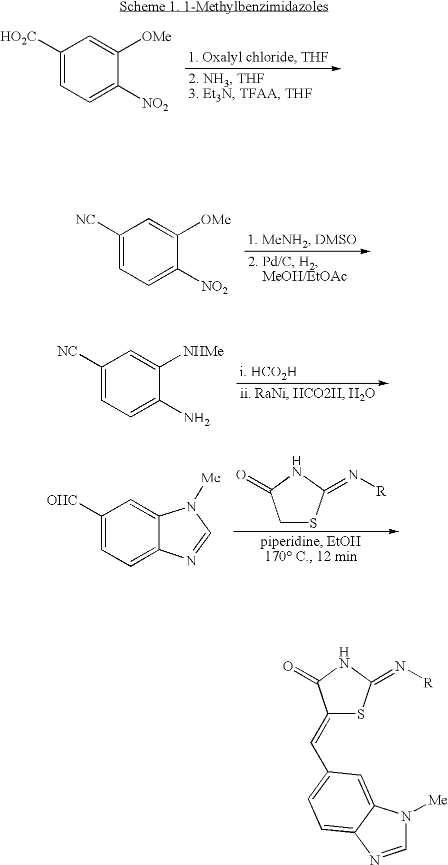

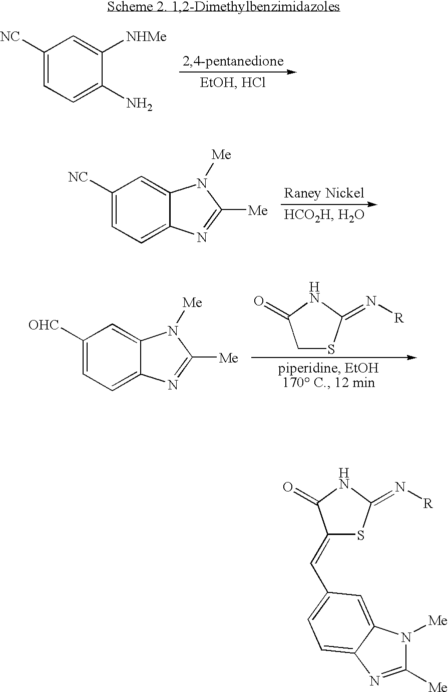

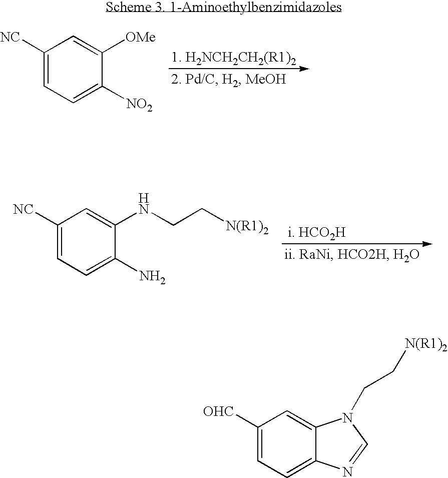

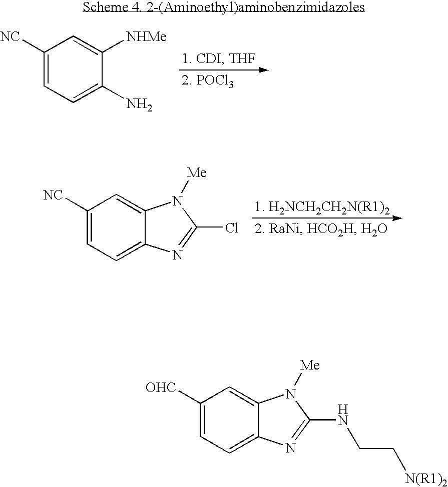

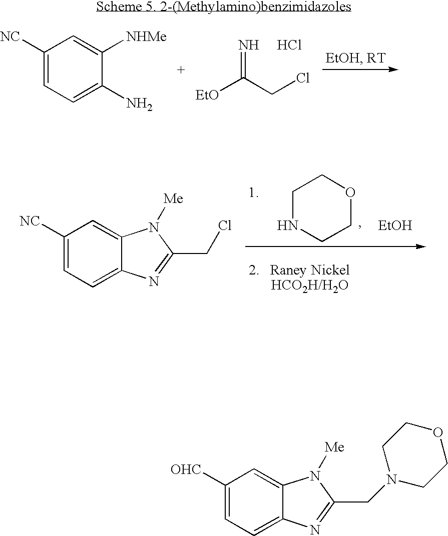

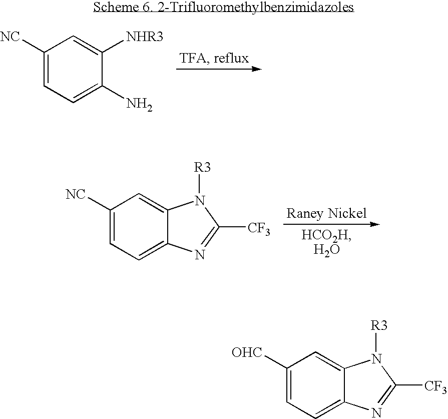

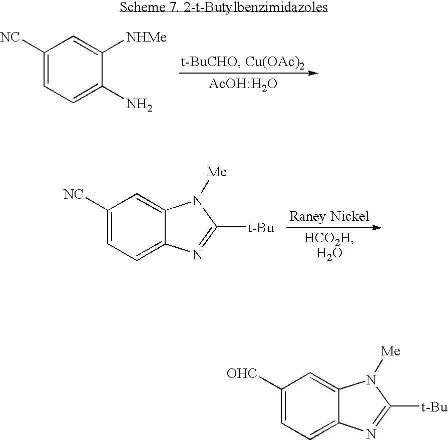

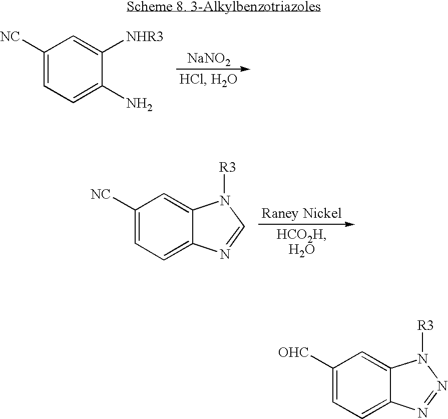

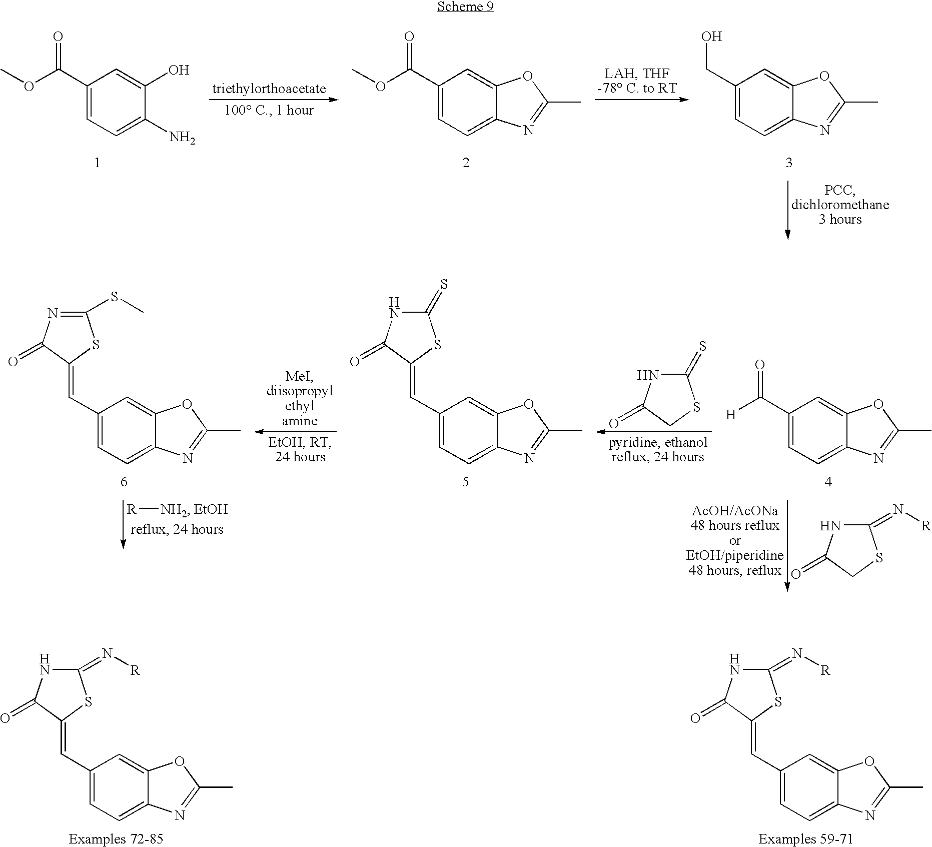

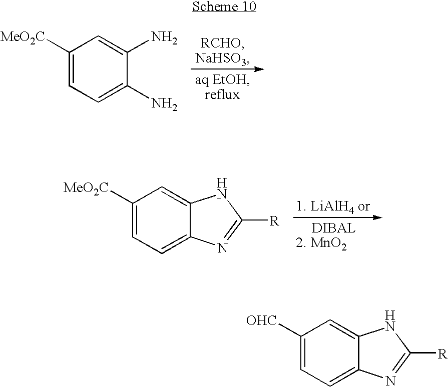

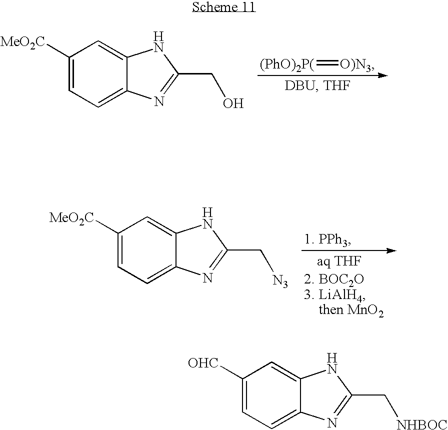

Briefly in Scheme 9, preparation of aldehyde 4 starts with cyclization of methyl 4-amino-3-hydroxy-benzoate 1. Benzoxazole 2 is formed by the reaction with triethylortho acetate. Other reagents, such us, but not limited to, acetamide, acetic anhydride, acetyl chloride, could be utilized in this reaction. Formed benzoxazole is then isolated from the reaction mixture by filtration. Reduction of the ester to the alcohol 3 is done using lithium aluminum hydride. Other reducing agents, such us, but limited to, DIBAL-H, diborane, sodium-ammonia, sodium borohydride can be used for this reaction. Oxidation of alcohol in the presence of PCC yields aldehyde 4. Other oxidative reagents, such us MnO₂ or Swern oxidation can be utilized in this case. Coupling of the aldehyde with thiazolidinone utilizing Knoevenagel reaction can proceed under acid or basis catalysis. When benzoxazole undergoes acid-catalyzed reaction, partial formation of the ring-opening product may be observed. Product is then purified by column chromatography. Coupling with rhodanine under basic conditions yields thiazolidinone 5, which was then methylated with MeI to give thiazolidinone 6. Other methylating agents suitable for this reaction are diazomethane, methyl sulfoxide or other suitable methylating agents. Displacement with a variety of alkyl and aryl amines is done in ethanol and pure product can be isolated by filtration.

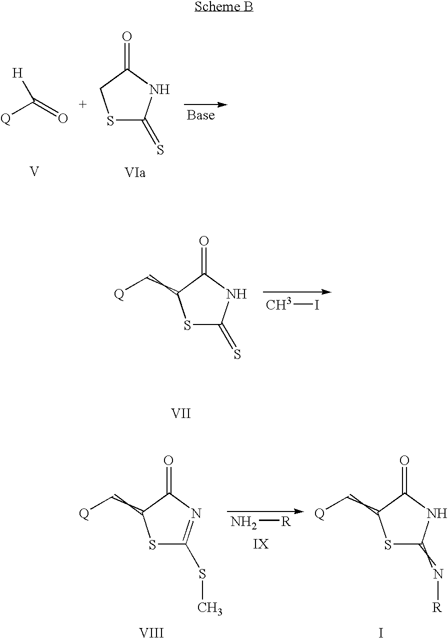

Scheme B is a variant of process of Scheme 9. Briefly in Scheme B, a mixture of an aldehyde of formula V (1 equivalent), rhodanine (1 equivalent), sodium acetate (about 3 equivalents), and acetic acid is heated at around 110° C. for about 48 h. The reaction mixture is cooled to room temperature to afford a product of formula VII.

Then, to a room temperature suspension of VII (1 equivalent) in a suitable solvent such as ethanol is added Hunig's base (about 2 equivalents) followed by iodomethane (about 5 equivalents). Stirring the resultant suspension at room temperature for 3.5 h will yield a compound of VIII. To a mixture of VIII (1 equivalent) and MS4A powder is added an amine of formula IX (1˜2 equivalent) and ethanol (dehydrated). The mixture was heated to afford a compound of formula I.

In the above Schemes, the meaning of R, R1, R3, and Q are as defined for Formula I.

### Biological Assays

The compounds of the present invention are tested to determine their inhibitory activity at PI3Kα, PI3Kδ, PI3Kβ and PI3Kγ according to the following.

For all PI3K isoforms:
1. Cloning, expression, purification, and characterization of the human Class Ia phosphoinositide 3-kinase isoforms: Meier, T. I.; Cook, J. A.; Thomas, J. E.; Radding, J. A.; Horn, C.; Lingaraj, T.; Smith, M. C. Protein Expr. Purif., 2004, 35(2), 218.
2. Competitive fluorescence polarization assays for the detection of phosphoinositide kinase and phosphatase activity: Drees, B. E.; Weipert, A.; Hudson, H.; Ferguson, C. G.; Chakravarty, L.; Prestwich, G. D. Comb. Chem. High Throughput. Screen., 2003, 6(4), 321.

### For PI3Kγ: WO 2005/011686 A1

### Experimental Details

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megahertz); | mol (moles); |
| mmol (millimoles); | rt (room temperature); |
| min (minutes); | h (hours); |
| mp (melting point); | TLC (thin layer chromatography); |
| Tr (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N-dimethylformamide); |
| DMPU (N,N′-dimethylpropyleneurea); | (CDl (1,1-carbonyldiimidazole); |
| IBCF (isobutyl chloroformate); | HOAc (acetic acid); |
| HOSu (N-hydroxysuccinimide); | HOBT (1-hydroxybenzotriazole); |
| mCPBA (meta-chloroperbenzoic acid; | EDC (ethylcarbodiimide hydrochloride); BOC |
| (tert-butyloxycarbonyl); | FMOC (9-fluorenylmethoxycarbonyl); DCC |
| (dicyclohexylcarbodiimide); | CBZ (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | BSA (bovine serum albumin) |
| ATP (adenosine triphosphate); | HRP (horseradish peroxidase); |
| DMEM (Dulbecco′s modified Eagle medium); | |
| HPLC (high pressure liquid chromatography); | |
| BOP (bis(2-oxo-3-oxazolidinyl)phosphinic | |
| chloride); | |
| TBAF (tetra-n-butylammonium fluoride); | |
| HBTU (O-Benzotriazole-1-yl-N,N,N′,N′- | |
| tetramethyluronium hexafluorophosphate). | |
| HEPES (4-(2-hydroxyethyl)-1-piperazine | |
| ethane sulfonic acid); | |
| DPPA (diphenylphosphoryl azide); | |
| fHNO3 (fumed HNO3); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### EXAMPLE 1

### (5Z)-2-[(2-Chlorophenyl)amino]-5-[(1-methyl-1H-benzimidazol-6-yl)methylidene]-1,3-thiazol-4(5H)-one

(a) 3-(Methyloxy)-4-nitrobenzonitrile. Following the procedure of Mackman et al. in J. Med. Chem. 2001, 44, 2753-2771, 3-methoxy-4-nitrobenzoic acid (11.52 g, 58.4 mmol) was dissolved in THF (158 mL) and cooled to 0° C. Oxalyl chloride (5.6 mL, 64.3 mmol) was added dropwise under a nitrogen atmosphere, followed by a few drops of DMF. The reaction mixture was allowed to warm to room temperature and stirred for 1 h. The mixture was then concentrated to dryness under reduced pressure, and the residue redissolved in THF (158 mL) and cooled to 0° C. Ammonia gas was bubbled through the solution for 10 min, leading to the formation of a yellow precipitate. The ice bath was removed, and the mixture was sealed and allowed to stir overnight. After the addition of EtOAc (100 mL), the solids were filtered off, washed with water, and dried to provide 3-(methyloxy)-4-nitrobenzamide (10.10 g, 88%) as a yellow solid. Additional product could be recovered from the filtrate by removal of the organic solvent under reduced pressure, then redissolving the residue in EtOAc. The organic layer was washed with 1N HCl (2×100 mL), brine (2×100 mL), then dried (Na₂SO₄), filtered and concentrated to afford an additional 1.07 g (9%). ¹H NMR (d₆-DMSO): □ 8.25 (bs, 1H), 7.96 (d, J=8.0 Hz, 1H), 7.75 (d, J=1.6 Hz, 1H), 7.73 (s, 1H), 7.57 (dd, J=1.6, 8.4 Hz, 1H), 3.98 (s, 3H).

To a suspension of 3-(methyloxy)-4-nitrobenzamide (11.17 g, 56.7 mmol) in THF (150 mL) was added Et₃N (10.3 mL, 73.7 mmol), followed by the dropwise addition of TFAA (8.67 mL, 62.4 mmol). After stirring for 1.5 h, the solvent was removed in vacuo and the mixture dissolved in EtOAc (400 mL). The solution was washed with 1N HCl (1×200 mL), brine (2×250 mL), dried over Na₂SO₄, filtered and concentrated to yield 3-(methyloxy)-4-nitrobenzonitrile (9.98 g, 96% overall) as a yellow solid. ¹H NMR (d₆-DMSO): □ 8.06 (d, J=8.4 Hz, 1H), 7.96 (d, J=1.2 Hz, 1H), 7.64 (dd, J=1.2, 8.4 Hz, 1H), 3.98 (s, 3H).

(b) 3-(Methylamino)-4-nitrobenzonitrile. Following the procedure of Mackman et al. in J. Med. Chem. 2001, 44, 2753-2771, 3-methoxy-4-nitrobenzonitrile (1.0 g, 5.62 mmol) was dissolved in DMSO (7 mL) in a pressure tube and a 40% solution of MeNH₂ in water (1 mL) was added. The tube was sealed and heated to 75° C. for 4 h, then cooled and poured onto an ice/water mixture. The precipitate was filtered, rinsed with water, and dried to afford 3-(methylamino)-4-nitrobenzonitrile (0.95 g, 95%) as an orange solid. ¹H NMR (CDCl₃): [symbol] 8.26 (d, J=8.8 Hz, 1H), 8.05 (bs, 1H), 7.15 (d, J=1.2 Hz, 1H), 6.89 (dd, J=1.6, 8.8 Hz, 1H), 3.06 (d, J=5.2 Hz, 3H).

(c) 4-Amino-3-(methylamino)benzonitrile. To a mixture of 3-(methylamino)-4-nitrobenzonitrile (0.655 g, 3.70 mmol) in MeOH (9.5 mL) and EtOAc (9.5 mL) was added 10% Pd/C (65 mg). After stirring under a hydrogen atmosphere for 4 h, the reaction mixture was filtered through a pad of Celite, rinsed with MeOH, and concentrated under reduced pressure to afford 4-amino-3-(methylamino)benzonitrile (0.542 g, 100%) as a beige solid. ¹H NMR (CDCl₃): [symbol] 7.02 (dd, J=1.6, 8.0 Hz, 1H), 6.84 (d, J=1.2 Hz, 1H), 6.68 (d, J=8.0 Hz, 1H), 3.74 (bs, 2H), 3.32 (bs, 1H), 2.87 (s, 3H).

(d) 1-Methyl-1H-benzimidazole-6-carbaldehyde. A mixture of 4-amino-3-(methylamino)benzonitrile (0.40 g, 2.72 mmol) in HCO₂H (9 mL) was heated to 100° C. for 2 h. The mixture of crude benzimidazole was then cooled, Raney nickel (0.4 g) and H₂O (2 mL) were added, and the mixture was heated again to 100° C. for 1 h. The hot mixture was then filtered through Celite, rinsed with MeOH and concentrated under reduced pressure. Water (1 mL) was added to the residue, which was then treated carefully with sat. aq. NaHCO₃. The solid which precipitated was filtered, rinsed with H₂O and dried to afford 1-methyl-1H-benzimidazole-6-carbaldehyde (0.412 g, 95%) as a tan solid, which was used directly in the next reaction. ¹H NMR (CDCl₃): [symbol] 10.12 (s, 1H), 8.05 (s, 1H), 8.00 (d, J=0.8 Hz, 1H), 7.92 (d, J=8.0 Hz, 1H), 7.84 (dd, J=1.2, 8.0 Hz, 1H), 3.94 (s, 3H).

(e) (5Z)-2-[(2-Chlorophenyl)amino]-5-[(1-methyl-1H-benzimidazol-6-yl)methylidene]-1,3-thiazol-4(5H)-one. A solution of 1-methyl-1H-benzimidazole-6-carbaldehyde (15 mg, 0.094 mmol), 2-[(2-chlorophenyl)amino]-1,3-thiazol-4(5H)-one (21.3 mg, 0.094 mmol), and piperidine (18.5 □L, 0.188 mmol) in EtOH (0.5 mL) was heated in a microwave reactor at 170° C. for 720 s. The solvent was then removed under reduced pressure and the crude product purified by precipitation from a mixture of CH₂Cl₂/hexanes. Alternatively, the product could be purified by column chromatography.

| Example | Compound name | NMR (400 MHz) |
|---|---|---|
| 1 | (5Z)-2-[(2- | (CDCl₃): 7.95 (s, 1H), 7.92 (s, 1H), |
| | Chlorophenyl)amino]- | 7.81 (d, J = 8.8 Hz, 1H), 7.48 (m, |
| | 5-[(1-methyl-1H- | 2H), 7.39 (dd, J = 0.8, 8.8 Hz, |
| | benzimidazol-6- | 1H), 7.31 (dt, J = 1.6, 7.6 Hz, |
| | yl)methylidene]-1,3- | 1H), 7.17 (dt, J = 1.6, 7.6 Hz, |
| | thiazol-4(5H)-one | 1H), 7.08 (m, 1H), 3.86 (s, 3H) |

### EXAMPLES 2-8

The following compounds were prepared according to the procedure of Example 1, except substituting the appropriately substituted thiazolone for 2-[(2-chlorophenyl)amino]-1,3-thiazol-4(5H)-one:

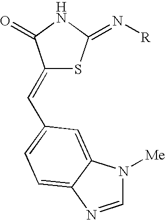

| Example | Compound name | R | NMR (400 MHz) |
|---|---|---|---|
| 2 | (5Z)-2-[(2,6-dichlorophenyl)amino]-5-[(1-methyl-1H-benzimidazol-6-yl)methylidene]-1,3-thiazol-4(5H)-one | 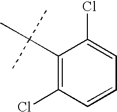 | (d₆-acetone): 8.11 (s, 1H),7.70 (m, 3 H), 7.43 (m, 3 H),7.12 (m, 1 H), 3.91 (s, 3 H) |
| 3 | (5Z)-2-[(2,6-difluorophenyl)amino]-5-[(1-methyl-1H-benzimidazol-6-yl)methylidene]-1,3-thiazol-4(5H)-one | 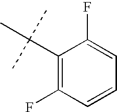 | (CDCl₃): 7.95 (s, 1 H), 7.92 (s,1 H), 7.81 (d, J = 8.0 Hz, 1 H),7.46 (s, 1 H), 7.38 (m, 1 H),7.12 (m, 1 H), 7.00 (t, J = 7.6Hz, 2 H), 3.89 (s, 3 H) |
| 4 | (5Z)-2-[(2,4-dimethylphenyl)amino]-5-[(1-methyl-1H-benzimidazol-6-yl)methylidene]-1,3-thiazol-4(5H)-one | 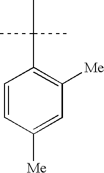 | (CDCl₃): 7.93 (s, 1 H), 7.90 (s,1 H), 7.80 (d, J = 7.6 Hz, 1 H),7.45 (s, 1 H), 7.39 (dd, J = 0.8,8.8 Hz, 1 H), 7.10 (s, 1 H), 7.05(dd, J = 2.0, 8.0 Hz, 1 H), 6.91(d, J = 8.8 Hz, 1 H), 3.89 (s,3 H), 2.36 (s, 3 H), 2.22 (s, 3 H) |
| 5 | (5Z)-5-[(1-methyl-1H-benzimidazol-6-yl)methylidene]-2-{[2-(methoxy)phenyl]amino}-1,3-thiazol-4(5H)-one | 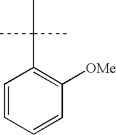 | (CDCl₃): 7.95 (s, 1 H), 7.84 (m,1 H), 7.51 (m, 2 H), 6.99 (m,2 H), 3.91 (s, 3 H), 3.89 (s, 3 H) |
| 6 | (5Z)-5-[(1-methyl-1H-benzimidazol-6-yl)methylidene]-2-{[2-(trifluoromethyl)-phenyl]-amino}-1,3-thiazol-4(5H)-one | 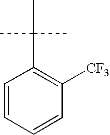 | (CDCl₃): 7.94 (s,1 H), 7.89 (s,1 H), 7.80 (dd, J = 0.8, 8.4 Hz,1 H), 7.71 (d, J = 7.6 Hz, 1 H),7.56 (m, 2 H), 7.45 (m, 1 H),7.38 (m, 1 H), 7.10 (m, 1 H),3.85 (s, 3 H) |
| 7 | (5Z)-2-[(2,4-difluorophenyl)amino]-5-[(1-methyl-1H-benzimidazol-6-yl)methylidene]-1,3-thiazol-4(5H)-one | 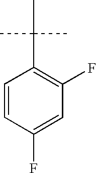 | (CDCl₃): 7.95 (s, 1 H), 7.88 (m,1 H), 7.43 (m, 1 H), 7.35 (m,2 H), 6.92 (m, 2 H), 3.74 (s,3 H), 2.63 (s, 3 H) |
| 8 | (5Z)-2-[(2-chloro-4-fluorophenyl)-amino]-5-[(1-methyl-1H-benzimidazol-6-yl)methylidene]-1,3-thiazol-4(5H)-one | 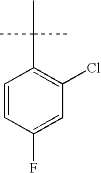 | (CD₃OD): 9.33 (s, 1 H), 8.01(s, 1 H), 7.88 (m, 2 H), 7.74 (dd,J = 1.6, 8.4 Hz, 1 H), 7.33 (d, J =8.4 Hz, 1 H), 7.11 (d, J = 6.4Hz, 2 H), 4.11 (s, 3 H) |

### EXAMPLE 9

### (5Z)-2-[(2-Chlorophenyl)amino]-5-[(1,2-dimethyl-1H-benzimidazol-6-yl)methylidene]-1,3-thiazol-4(5H)-one

(a) 1,2-Dimethyl-1H-benzimidazole-6-carbonitrile. 4-Amino-3-(methylamino)benzonitrile (from Example 1(c); 0.500 g, 3.40 mmol) and 2,4-pentanedione (0.700 mL, 6.80 mmol) were dissolved in EtOH (8.4 mL) and cooled to 0° C. 6N HCl (2.8 mL) was added dropwise and the solution turned deep red. Stirring was continued for 20 min, after which time the mixture was carefully poured onto ice/sat. aq. NaHCO₃, making sure the aqueous layer remained basic. The solid product which precipitated was filtered off, rinsed with H₂O and dried to afford the crude 1,2-dimethyl-1H-benzimidazole-6-carbonitrile (0.355 g, 61%). ¹H NMR (CDCl₃): [symbol] 7.72 (d, J=8.4 Hz, 1H), 7.62 (d, J=1.2 Hz, 1H), 7.50 (dd, J=1.2, 8.4 Hz, 1H), 3.78 (s, 3H), 2.66 (s, 3H).

(b) 1,2-Dimethyl-1H-benzimidazole-6-carbaldehyde. A mixture of 1,2-dimethyl-1H-benzimidazole-6-carbonitrile (0.355 g, 2.08 mmol) and Raney nickel (150 mg) was suspended in HCO₂H (7 mL) and H₂O (3 mL) and heated to 100° C. for 2 h. The mixture was then filtered hot through Celite, rinsed with MeOH, and concentrated. To the resulting residue was added H₂O (1 mL) followed by sat. aq. NaHCO₃ until basic. The aqueous layer was extracted with CH₂Cl₂ (2×30 mL), dried over Na₂SO₄, and concentrated to yield the product aldehyde (0.284 g, 79%) as a beige solid. ¹H NMR (CDCl₃): [symbol] 10.07 (s, 1H), 7.88 (t, J=1.2H, 1H), 7.77 (d, J=1.2 Hz, 2H), 3.81 (s, 3H), 2.61 (s, 3H).

(c) (5Z)-2-[(2-Chlorophenyl)amino]-5-[(1,2-dimethyl-1H-benzimidazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 9 | (5Z)-2-[(2-Chlorophenyl)- | (d₆-DMSO): 12.58 (s, 1H), |
| | amino]-5- | 7.81 (s, 1H), 7.72 (s, 1H), 7.59 |
| | [(1,2-dimethyl-1H- | (d, J = 8.4 Hz, 1H), 7.55 (d, |
| | benzimidazol-6-yl)- | J = 8.0 Hz, 1H), 7.38 (m, 1H), |
| | methylidene]- | 7.23 (m, 3H), 3.72 (s, 3H), |
| | 1,3-thiazol-4(5H)-one | 2.53 (s, 3H) |

### EXAMPLES 10-14

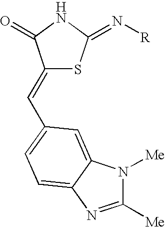

The following compounds were prepared according to the procedure of Example 9, except substituting the appropriate thiazolone for 2-[(2-chlorophenyl)amino]-1,3-thiazol-4(5H)-one:

| Example | Compound name | R | NMR (400 MHz) |
|---|---|---|---|
| 10 | (5Z)-2-[(2,6-dichlorophenyl)amino]-5-[(1,2-dimethyl-1H-benzimidazol-6-yl)methylidene]-1,3-thiazol-4(5H)-one | 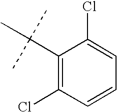 | (d₆-DMSO): 7.57 (s, 1 H), 7.52(d, J = 7.6 Hz, 1 H), 7.41 (m,2 H), 7.26 (m, 1 H), 6.97 (m,1 H), 3.29 (s, 3 H), 2.5 (s, 3 H) |
| 11 | (5Z)-2-[(2,6-difluorophenyl)amino]-5-[(1,2-dimethyl-1H-benzimidazol-6-yl)methylidene]-1,3-thiazol-4(5H)-one | 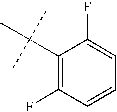 | (CD₃OD): 7.85 (s, 1 H), 7.56(m, 2 H), 7.33 (dd, J = 1.2, 8.4Hz, 1 H), 7.20 (m, 1 H), 7.05 (t,J = 8.0 Hz, 2 H), 3.76 (s, 3 H),2.59 (s, 3 H) |
| 12 | (5Z)-5-[(1,2-dimethyl-1H-benzimidazol-6-yl)methylidene]-2-[(2,4-dimethylphenyl)amino]-1,3-thiazol-4(5H)-one | 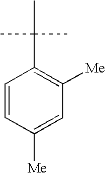 | (d₆-DMSO): 7.76 (d, J = 3.6Hz, 1 H), 7.71 (s, 1 H), 7.58 (d,J = 8.4 Hz, 1 H), 7.22 (dd, J =1.6, 8.4 Hz, 1 H), 7.10 (s, 1 H),7.02 (m, 1 H), 3.71 (s, 3 H),3.29 (s, 3 H), 2.53 (s, 3 H), 2.29(s, 3 H) |
| 13 | (5Z)-2-[(2,4-difluorophenyl)amino]-5-[(1,2-dimethyl-1H-benzimidazol-6-yl)methylidene]-1,3-thiazol-4(5H)-one | 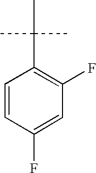 | (CD₃OD): 7.83 (s, 1 H), 7.58(m, 2 H), 7.36 (m, 1 H), 7.06(m, 3 H), 3.77 (s, 3 H), 2.60 (s,3 H) |
| 14 | (5Z)-2-[(2-chloro-4-fluorophenyl)amino]-5-[(1,2-dimethyl-1H-benzimidazol-6-yl)methylidene]-1,3-thiazol-4(5H)-one | 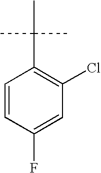 | (CD₃OD): 7.82 (s, 1 H), 7.57(m, 2 H), 7.35 (dd, J = 0.8, 8.8Hz, 1 H), 7.31 (d, J = 8.8 hz,1 H), 7.12 (d, J = 5.6 Hz, 2 H),3.77 (s, 3 H), 2.60 (s, 3 H) |

### EXAMPLE 15

### (5Z)-2-[(2-Chlorophenyl)amino]-5-({1-[2-(4-morpholinyl)ethyl]-1H-benzimidazol-6-yl}methylidene)-1,3-thiazol-4(5H)-one

(a) 4-Amino-3-{[2-(4-morpholinyl)ethyl]amino}benzonitrile. A mixture of 3-methoxy-4-nitrobenzonitrile (from Example 1(a); 0.178 g, 1.0 mmol) and 4-(2-aminoethyl)morpholine (0.65 mL, 5.0 mmol) were heated to 80° C. for 20 h. The reaction mixture was cooled, diluted with EtOAc (50 mL), and washed with H₂O (3×30 mL) and brine (1×30 mL). The organic layer was dried (Na₂SO₄), filtered, and concentrated to afford 3-{[2-(4-morpholinyl)ethyl]amino}-4-nitrobenzonitrile (0.246 g, 89%) as a red solid. This material was dissolved in MeOH (3 mL), 10% Pd/C (24 mg) was added, and the mixture stirred under a hydrogen atmosphere overnight. Filtration through Celite and removal of the solvent provided the desired diaminobenzonitrile (0.219 g, 100%) as a red oil.

(b) 1-[2-(4-Morpholinyl)ethyl]-1H-benzimidazole-6-carbaldehyde. According to the procedure in Example 1(d), 4-amino-3-{[2-(4-morpholinyl)ethyl]amino}benzonitrile was converted to the benzimidazole carboxaldehyde in 72% yield. ¹H NMR (CDCl₃): [symbol] 10.11 (s, 1H), 8.20 (s, 1H), 8.02 (d, J=0.8 Hz, 1H), 7.92 (d, J=8.4 Hz, 1H), 7.82 (dd, J=1.2, 8.4 Hz, 1H), 4.34 (t, J=6.0 Hz, 2H), 3.69 (t, J=4.4 Hz, 4H), 2.98 (t, J=6.0 Hz, 2H), 2.50 (t, J=4.8 Hz, 4H).

(c) (5Z)-2-[(2-Chlorophenyl)amino]-5-({1-[2-(4-morpholinyl)ethyl]-1H-benzimidazol-6-yl}methylidene)-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 15 | (5Z)-2-[(2-Chlorophenyl)- | (CD₃OD): 8.30 (s, 1H), 7.84 (s, 1H), 7.72 (m, |
| | amino]-5- | 2H), 7.48 (dd, J = 1.6, 8.0 Hz, 1H), 7.41 (m, |
| | ({1-[2-(4-morpholinyl)ethyl]- | 1H), 7.33 (dt, J = 1.6, 7.6 Hz, 1H), 7.19 (dt, J = 1.2, |
| | 1H-benzimidazol-6-yl}- | 7.6 Hz, 1H), 7.09 (m, 1H), 4.39 (t, J = 6.4 Hz, |
| | methylidene)-1,3-thiazol- | 2H), 3.59 (m, 4H), 2.74 (t, J = 6.4 Hz, 2H), |
| | 4(5H)-one | 2.43 (m, 4H) |

### EXAMPLES 16-32

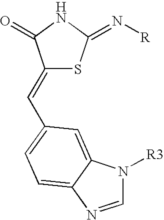

The following compounds were prepared according to the procedure of Example 15, using the requisite amine for 4-(2-aminoethyl)morpholine and substituting the appropriate thiazolone for 2-[(2-chlorophenyl)amino]-1,3-thiazol-4(5H)-one:

| | Compound | | | |
|---|---|---|---|---|
| Example | name | R3 | R | NMR (400 MHz) |
| 16 | (5Z)-2-[(2,6-dichlorophenyl)amino]-5-({1-[2-(4-morpholinyl)ethyl]-1H-benzimidazol-6-yl}methylidene)-1,3-thiazol-4(5H)-one | 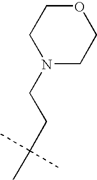 | 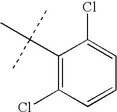 | (CD₃OD): 8.30 (s, 1 H), 7.87 (s,1 H), 7.72 (m, 2 H), 7.44 (d, J =8.0 Hz, 2 H), 7.39 (d, J = 8.0 Hz,1 H), 7.15 (m, 1 H), 4.39 (t, J = 5.6Hz, 2 H), 3.60 (m, 4 H), 2.74 (t, J =5.6 Hz, 2 H), 2.44 (m, 4 H) |
| 17 | (5Z)-2-[(2-chloro-4-fluorophenyl)amino]-5-({1-[2-(4-morpholinyl)ethyl]-1H-benzimidazol-6-yl}methylidene)-1,3-thiazol-4(5H)-one | 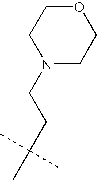 | 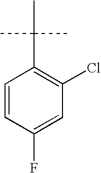 | (CD₃OD): 8.30 (s, 1 H), 7.85 (s,1 H), 7.72 (m, 2 H), 7.41 (dd, J =1.2, 7.6 Hz, 1 H), 7.33 (dd, J =1.2, 9.2 Hz, 1 H), 7.12 (d, J = 7.2Hz, 2 H), 4.40 (t, J = 6.4 Hz, 2 H),3.59 (m, 4 H), 2.75 (t, J = 6.0 Hz,2 H), 2.44 (m, 4 H) |
| 18 | (5Z)-2-[(2-chlorophenyl)amino]-5-({1-[2-(dimethylamino)ethyl]-1H-benzimidazol-6-yl}methylidene)-1,3-thiazol-4(5H)-one | 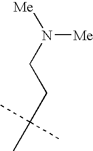 | 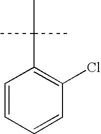 | (CD₃OD): 8.28 (s, 1 H), 7.84 (s,1 H), 7.74 (s, 1 H), 7.70 (d, J = 8.4Hz, 1 H), 7.47 (d, J = 8.4 Hz, 1 H),7.41 (d, J = 8.4 Hz, 1 H), 7.32 (t,J = 7.6 Hz, 1 H), 7.18 (t, J = 7.6Hz, 1 H), 7.09 (d, J = 7.6 Hz, 1 H),4.37 (t, J = 6.8 Hz, 2 H), 2.73 (t, J =6.8 Hz, 2 H) |
| 19 | (5Z)-2-[(2,6-dichlorophenyl)amino]-5-({1-[2-(dimethylamino)ethyl]-1H-benzimidazol-6-yl}methylidene)-1,3-thiazol-4(5H)-one | 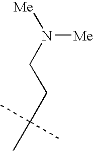 | 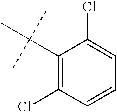 | (CD₃OD): 8.24 (s, 1 H), 7.71 (m,3 H), 7.40 (m, 3 H), 7.10 (t, J = 8.0Hz, 1 H), 4.35 (t, J = 6.4 Hz, 2 H),2.71 (t, J = 6.4 Hz, 2 H), 2.19 (s,6 H). |
| 20 | (5Z)-2-[(2,4-difluorophenyl)amino]-5-({1-[2-(dimethylamino)ethyl]-1H-benzimidazol-6-yl}methylidene)-1,3-thiazol-4(5H)-one | 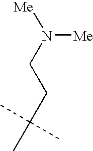 | 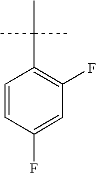 | (CD₃OD): 8.30 (s, 1 H), 7.84 (s,1 H), 7.74 (s, 1 H), 7.71 (d, J = 8.4Hz, 1 H), 7.43 (dd, J = 1.2, 8.8Hz, 1 H), 7.10 (m, 2 H), 6.98 (m,1 H), 4.39 (t, J = 6.8 Hz, 2 H),2.75 (t, J = 6.8 Hz, 2 H), 2.23 (s,6 H) |
| 21 | (5Z)-5-({1-[2-(dimethylamino)ethyl]-1H-benzimidazol-6-yl}methylidene)-2-(phenylamino)-1,3-thiazol-4(5H)-one | 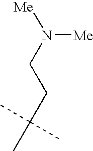 | 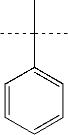 | (CD₃OD, mixture of twoisomers): 8.35 (s, 1 H), 8.29 (s,1 H), 7.98 (s, 1 H), 7.70-7.88 (m,8 H), 7.57 (m, 1 H), 7.41 (m, 5 H),7.23 (m, 2 H), 7.10 (d, J = 7.6 Hz,2 H), 4.48 (m, 2 H), 4.38 (m, 2 H),2.86 (m, 2 H), 2.74 (m, 2 H), 2.35(s, 6 H), 2.22 (s, 6 H) |
| 22 | (5Z)-2-[(2-chlorophenyl)amino]-5-({1-[2-(diethylamino)ethyl]-1H-benzimidazol-6-yl}methylidene)-1,3-thiazol-4(5H)-one | 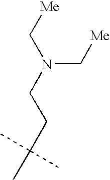 | 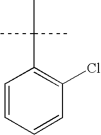 | (CD₃OD): 8.27 (s, 1 H), 7.84 (s,1 H), 7.71 (m, 2 H), 7.48 (dd, J =1.6, 8.0 Hz, 1 H), 7.41 (d, J = 8.4Hz, 1 H), 7.32 (t, J = 8.0 Hz, 1 H),7.18 (t, J = 7.6 Hz, 1 H), 7.08 (dd,J = 0.8, 8.0 Hz, 1 H), 4.32 (t, J =6.0 Hz, 2 H), 2.80 (t, J = 6.4 Hz,2 H), 2.48 (q, J = 7.2 Hz, 4 H),0.86 (t, J = 7.2 Hz, 6 H) |
| 23 | (5Z)-2-[(2,6-dichlorophenyl)amino]-5-({1-[2-(diethylamino)ethyl]-1H-benzimidazol-6-yl}methylidene)-1,3-thiazol-4(5H)-one | 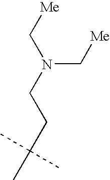 | 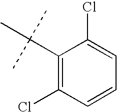 | (CD₃OD): 8.27 (s, 1 H), 7.85 (s,1 H), 7.70 (m, 2 H), 7.43 (d, J =8.0 Hz, 2 H), 7.40 (d, J = 8.8 Hz,1 H), 7.14 (t, J = 8.0 Hz, 1 H),4.32 (t, J = 6.4 Hz, 2 H), 2.80 (t, J =6.4 Hz, 2 H), 2.47 (q, J = 6.8Hz, 4 H), 0.86 (t, J = 6.8 Hz, 6 H) |
| 24 | (5Z)-2-[(2-chlorophenyl)amino]-5-({1-[3-(4-morpholinyl)propyl]-1H-benzimidazol-6-yl}methylidene)-1,3-thiazol-4(5H)-one | 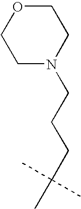 | 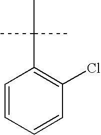 | (CD₃OD): 8.29 (s, 1 H), 7.86 (s,1 H), 7.73 (s, 1 H), 7.71 (d, J = 8.4Hz, 1 H), 7.48 (d, J = 7.6 Hz, 1 H),7.40 (d, J = 8.4 Hz, 1 H), 7.33 (t,J = 7.6 Hz, 1 H), 7.19 (t, J = 7.6Hz, 1 H), 7.09 (d, J = 7.6 Hz, 1 H),4.36 (t, J = 6.8 Hz, 2 H), 3.60 (bs,4 H), 2.32 (bs, 4 H), 2.26 (t, J =6.8 Hz, 2 H), 2.04 (t, J = 6.8 Hz,2 H) |
| 25 | (5Z)-2-[(2,6-dichlorophenyl)amino]-5-({1-[3-(4-morpholinyl)propyl]-1H-benzimidazol-6-yl}methylidene)-1,3-thiazol-4(5H)-one | 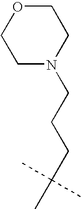 | 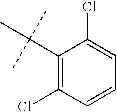 | (CD₃OD): 9.09 (m, 1 H), 8.01 (bs,1 H), 7.91 (s, 1 H), 7.85 (m, 1 H),7.61 (m, 1 H), 7.45 (d, J = 8.0 Hz,2 H), 7.16 (t, J = 8.0 Hz, 1 H),4.58 (bs, 2 H), 4.02 (m, 2 H), 3.75(m, 2 H), 3.47 (m, 2 H), 3.25 (bs,2 H), 3.12 (m, 2 H), 2.42 (bs, 2 H) |
| 26 | (5Z)-2-[(2-chlorophenyl)amino]-5-({1-[3-(4-methyl-1-piperazinyl)propyl]-1H-benzimidazol-6-yl}methylidene)-1,3-thiazol-4(5H)-one | 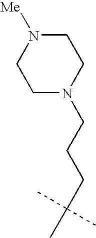 | 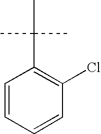 | (CD₃OD): 8.27 (s, 1 H), 7.81 (s,1 H), 7.74 (s, 1 H), 7.70 (d, J = 8.8Hz, 1 H), 7.47 (d, J = 8.0 Hz, 1 H),7.39 (d, J = 8.4 Hz, 1 H), 7.32 (t,J = 7.2 Hz, 1 H), 7.18 (t, J = 7.2Hz, 1 H), 7.09 (d, J = 7.6 Hz, 1 H),4.34 (t, J = 6.4 Hz, 2 H), 2.46 (bs,8 H), 2.30 (s, 3 H), 2.27 (t, J = 6.4Hz, 2 H), 2.03 (t, J = 6.4 Hz, 2 H) |
| 27 | (5Z)-2-[(2,6-dichlorophenyl)amino]-5-({1-[3-(4-methyl-1-piperazinyl)propyl]-1H-benzimidazol-6-yl}methylidene)-1,3-thiazol-4(5H)-one | 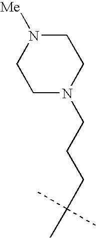 | 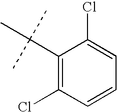 | (CD₃OD): 9.37 (s, 1 H), 8.09 (s,1 H), 7.93 (s, 1 H), 7.89 (d, J = 8.4Hz, 1 H), 7.69 (d, J = 8.0 Hz, 1 H),7.35 (d, J = 8.4 Hz, 2 H), 7.16 (t,J = 8.0 Hz, 1 H), 4.59 (m, 2 H),2.89 (s, 3 H), 2.83 (m, 8 H), 2.62(bs, 2 H), 2.22 (t, J = 6.4 Hz, 2 H) |
| 28 | (5Z)-2-[(2-chlorophenyl)amino]-5-({1-[2-(1-pyrrolidinyl)ethyl]-1H-benzimidazol-6-yl}methylidene)-1,3-thiazol-4(5H)-one | 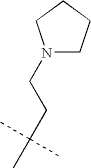 | 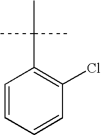 | (CD₃OD): 8.29 (s, 1 H), 7.84 (s,1 H), 7.71 (m, 2 H), 7.47 (dd, J =0.8, 8.0 Hz, 1 H), 7.41 (d, J = 8.0Hz, 1 H), 7.32 (dt, J = 0.8, 7.6 Hz,1 H), 7.18 (dt, J = 0.8, 7.6 Hz,1 H), 7.09 (dd, J = 1.6, 8.0 Hz,1 H), 4.40 (t, J = 6.4 Hz, 2 H),2.93 (t, J = 6.8 Hz, 2 H), 2.53 (bs,4 H), 1.76 (bs, 4 H) |
| 29 | (5Z)-2-[(2,6-dichlorophenyl)amino]-5-({1-[2-(1-pyrrolidinyl)ethyl]-1H-benzimidazol-6-yl}methylidene)-1,3-thiazol-4(5H)-one | 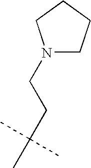 | 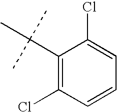 | (CD₃OD): 8.30 (s, 1 H), 7.87 (s,1 H), 7.72 (m, 2 H), 7.44 (d, J =8.0 Hz, 2 H), 7.40 (dd, J = 0.4,9.2 Hz, 1 H), 7.14 (t, J = 8.0 Hz,1 H), 4.43 (t, J = 6.4 Hz, 2 H),2.96 (m, 2 H), 2.56 (bs, 4 H), 1.78(bs, 4 H) |
| 30 | (5Z)-2-[(2-chlorophenyl)amino]-5-({1-[2-(1-piperidinyl)ethyl]-1H-benzimidazol-6-yl}methylidene)-1,3-thiazol-4(5H)-one | 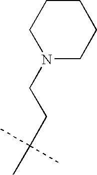 | 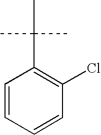 | (CD₃OD): 8.29 (s, 1 H), 7.89 (s,1 H), 7.85 (m, 2 H), 7.47 (d, J =6.8 Hz, 1 H), 7.41 (d, J = 7.2 Hz,1 H), 7.33 (t, J = 8.0 Hz, 1 H),7.19 (t, J = 7.6 Hz, 1 H), 7.09 (d,J = 8.0 Hz, 1 H), 4.40 (2 H, 6.4 Hz,2 H), 2.74 (t, J = 6.4 Hz, 2 H),2.44 (bs, 4 H), 1.54 (m, 6 H) |
| 31 | (5Z)-2-[(2,6-dichlorophenyl)amino]-5-({1-[2-(1-piperidinyl)ethyl]-1H-benzimidazol-6-yl}methylidene)-1,3-thiazol-4(5H)-one | 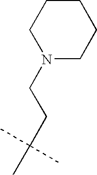 | 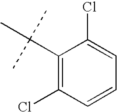 | (CD₃OD): 8.29 (s, 1 H), 7.86 (s,1 H), 7.71 (m, 2 H), 7.44 (d, J =8.0 Hz, 2 H), 7.41 (d, J = 8.8 Hz,1 H), 7.14 (t, J = 8.0 Hz, 1 H),4.40 (t, J = 6.0 Hz, 2 H), 2.73 (t, J =6.0 Hz, 2 H), 2.44 (bs, 4 H),1.54 (m, 6 H) |
| 32 | (5Z)-2-[(2,6-difluorophenyl)amino]-5-({1-[2-(1-piperidinyl)ethyl]-1H-benzimidazol-6-yl}methylidene)-1,3-thiazol-4(5H)-one | 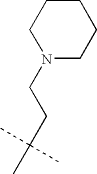 | 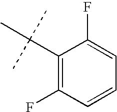 | (CD₃OD): 8.32 (s, 1 H), 7.84 (s,1 H), 7.80 (s, 1 H), 7.70 (d, J = 8.8Hz, 1 H), 7.38 (d, J = 8.4 Hz, 1 H),7.21 (m, 1 H), 7.05 (t, J = 8.0 Hz,2 H), 4.50 (t, J = 6.8 Hz, 2 H),2.90 (t, J = 6.8 Hz, 2 H), 2.60 (bs,4 H), 1.62 (m, 4 H), 1.49 (m, 2 H) |

### EXAMPLE 33

### (5Z)-2-[(2-Chlorophenyl)amino]-5-({1-[2-(dimethylamino)ethyl]-2-methyl-1H-benzimidazol-6-yl}methylidene)-1,3-thiazol-4(5H)-one

4-Amino-3-{[2-(dimethylamino)ethyl]amino}benzonitrile. Following the procedure outlined in Example 15(a), 3-methoxy-4-nitrobenzonitrile was converted into 4-amino-3-{[2-(dimethylamino)ethyl]amino}benzonitrile in quantitative yield using N,N-dimethylethylenediamine as the nucleophile. ¹H NMR (CDCl₃): [symbol] 7.01 (dd, J=1.6, 8.0 Hz, 1H), 6.80 (d, J=1.6 Hz, 1H), 6.65 (d, J=8.0 Hz, 1H), 3.98 (m, 2H), 3.12 (m, 2H), 2.62 (t, J=6.0 Hz, 2H), 2.26 (s, 3H).

(b) 1-[2-(Dimethylamino)ethyl]-2-methyl-1H-benzimidazole-6-carbaldehyde. The title compound was synthesized according to the procedure in Example 9(a) and 9(b), starting from 4-amino-3-{[2-(dimethylamino)ethyl]amino}benzonitrile. ¹H NMR (CDCl₃): [symbol] 10.07 (s, 1H), 7.88 (d, J=1.2 Hz, 1H), 7.77 (s, 2H0, 4.25 (m, 2H), 2.68 (s, 3H), 2.67 (m, 2H), 2.31 (s, 6H).

(c) (5Z)-2-[(2-Chlorophenyl)amino]-5-({1-[2-(dimethylamino)ethyl]-2-methyl-1H-benzimidazol-6-yl}methylidene)-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 33 | (5Z)-2-[(2- | (CD₃OD): 7.81 (s, 1H), 7.60 (m, 2H), 7.47 (d, J = 8.0 Hz, |
| | Chlorophenyl)amino]-5-({1- | 1H), 7.36 (d, J = 8.0 Hz, 1H), 7.32 (t, J = 7.6 Hz, |
| | [2-(dimethylamino)ethyl]-2- | 1H), 7.18 (t, J = 7.6 Hz, 1H), 7.08 (d, J = 7.6 Hz, |
| | methyl-1H-benzimidazol-6- | 1H), 4.28 (t, J = 7.2 Hz, 2H), 2.64 (t, J = 7.2 Hz, |
| | yl}methylidene)-1,3-thiazol- | 2H), 2.61 (s, 3H), 2.21 (s, 6H) |
| | 4(5H)-one | |

### EXAMPLES 34-40

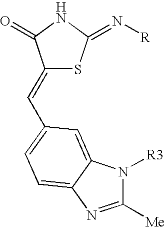

The following compounds were prepared according to the procedure of Example 33, using the requisite amine for N,N-dimethylethylenediamine and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 34 | (5Z)-2-[(2,6-dichlorophenyl)amino]-5-({1-[2-(dimethylamino)ethyl]-2-methyl-1H-benzimidazol-6-yl}methylidene)-1,3-thiazol-4(5H)-one | 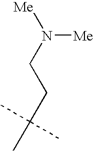 | 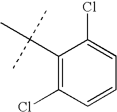 | (CD₃OD): 7.84 (s, 1 H), 7.58(m, 2 H), 7.44 (d, J = 8.0 Hz,2 H), 7.36 (d, J = 8.8 Hz, 1 H),7.15 (t, J = 8.0 Hz, 1 H), 4.28(t, J = 6.8 Hz, 2 H), 2.64 (t, J =6.8 Hz, 2 H), 2.62 (s, 3 H), 2.21(s, 6 H) |
| 35 | (5Z)-2-[(2,4-difluorophenyl)amino]-5-({1-[2-(dimethylamino)ethyl]-2-methyl-1H-benzimidazol-6-yl}methylidene)-1,3-thiazol-4(5H)-one | 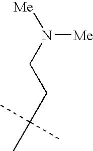 | 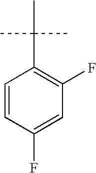 | (CD₃OD): 7.84 (s, 1 H), 7.61(m, 2 H), 7.38 (dd, J = 1.2, 8.0Hz, 1 H), 6.99-7.14 (m, 3 H),4.34 (t, J = 7.6 Hz, 2 H), 2.74(t, J = 7.6 Hz, 2 H), 2.63 (s,3 H), 2.31 (s, 6 H) |
| 36 | (5Z)-5-({1-[2-(dimethylamino)ethyl]-2-methyl-1H-benzimidazol-6-yl}methylidene)-2-(phenylamino)-1,3-thiazol-4(5H)-one | 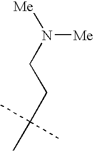 | 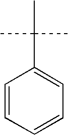 | (CD₃OD, mixture of twoisomers): 7.09-7.95 (m, 9 H),4.28-4.39 (m, 2 H), 2.61-2.76(m, 5 H), 2.37 (s, 3 H), 2.23 (s,3 H) |
| 37 | (5Z)-2-[(2,6-dichlorophenyl)amino]-5-{[1-(2-hydroxyethyl)-2-methyl-1H-benzimidazol-6-yl]methylidene}-1,3-thiazol-4(5H)-one | 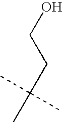 | 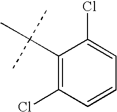 | (CD₃OD): 7.85 (s, 1 H), 7.63(s, 1 H), 7.58 (d, J = 7.6 Hz,1 H), 7.35 (d, J = 8.0 Hz, 2 H),7.32 (d, J = 8.0 Hz, 1 H), 7.15(t, J = 8.0 Hz, 1 H), 4.32 (t, J =4.4 Hz, 2 H), 3.85 (t, J = 4.4Hz, 2 H), 2.64 (s, 3 H) |
| 38 | (5Z)-2-[(2-chlorophenyl)amino]-5-({2-methyl-1-[2-(1-piperidinyl)ethyl]-1H-benzimidazol-6-yl}methylidene)-1,3-thiazol-4(5H)-one | 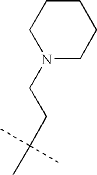 | 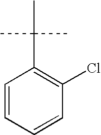 | (CD₃OD): 7.83 (s, 1 H), 7.60(m, 2 H), 7.48 (d, J = 8.0 Hz,1 H), 7.44 (m, 2 H), 7.18 (t, J =8.0 Hz, 1 H), 7.09 (d, J = 7.2Hz, 1 H), 4.31 (t, J = 6.4 Hz,2 H), 2.65 (t, J = 6.4 Hz, 2 H),2.63 (s, 3 H), 2.43 (bs, 4 H),1.54 (m, 4 H), 1.46 (m, 2 H) |
| 39 | (5Z)-2-[(2,6-dichlorophenyl)amino]-5-({2-methyl-1-[2-(1-piperidinyl)ethyl]-1H-benzimidazol-6-yl}methylidene)-1,3-thiazol-4(5H)-one | 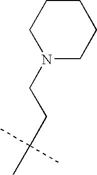 | 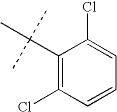 | (CD₃OD): 7.85 (s, 1 H), 7.68(m, 2 H), 7.44 (d, J = 8.4 Hz,2 H), 7.34 (dd, J = 1.2, 8.0 Hz,1 H), 7.14 (t, J = 8.0 Hz, 1 H),4.31 (t, J = 6.4 Hz, 2 H), 2.65(t, J = 6.0 Hz, 2 H), 2.63 (s,3 H), 2.44 (bs, 4 H), 1.54 (m,4 H), 1.46 (m, 2 H) |
| 40 | (5Z)-2-[(2,6-difluorophenyl)amino]-5-({2-methyl-1-[2-(1-piperidinyl)ethyl]-1H-benzimidazol-6-yl}methylidene)-1,3-thiazol-4(5H)-one | 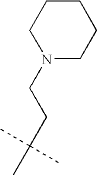 | 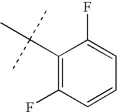 | (CD₃OD): 7.85 (s, 1 H), 7.59(m, 2 H), 7.35 (dd, J = 1.2, 8.4Hz, 1 H), 7.19 (m, 1 H), 7.05 (t,J = 8.0 Hz, 2 H), 4.33 (t, J =6.8 Hz, 2 H), 2.69 (t, J = 6.8Hz, 2 H), 2.63 (s, 3 H), 2.47 (bs,1 H), 1.55 (m, 4 H), 1.47 (m,2 H) |

### EXAMPLE 41

### (5Z)-2-[(2-Chlorophenyl)amino]-5-[(1-methyl-2-{[2-(4-morpholinyl)ethyl]amino}-1H-benzimidazol-6-yl)methylidene]-1,3-thiazol-4(5H)-one

(a) 3-Methyl-2-oxo-2,3-dihydro-1H-benzimidazole-5-carbonitrile. A suspension of 4-amino-3-(methylamino)benzonitrile (0.500 g, 3.40 mmol) and 1,1′-carbonyldiimidazole (1.10 g, 6.80 mmol) in THF (8.5 mL) was stirred at room temperature for 2 days. The reaction mixture was filtered, the collected solid rinsed with small amounts of H₂O and EtOAc, and dried to afford the cyclic urea (0.489 g, 83%) as a pink solid. Additional material could be recovered from the filtrate by separating the organic layer, washing with 0.1N HCl (1×30 mL), brine (1×30 mL), and drying over Na₂SO₄. After filtration, removal of solvent yielded 40 mg (7%) of additional product. ¹H NMR (d₆-acetone): [symbol] 10.23 (bs, 1H), 7.46 (s, 1H), 7.42 (dd, J=1.6, 8.0 Hz, 1H), 7.20 (d, J=8.0 Hz, 1H), 3.40 (s, 3H).

(b) 2-Chloro-1-methyl-1H-benzimidazole-6-carbonitrile. A mixture of 3-methyl-2-oxo-2,3-dihydro-1H-benzimidazole-5-carbonitrile (0.219 g, 1.26 mmol) in POCl₃ (2.5 mL) was heated to 105° C. overnight. Upon cooling, the excess POCl₃ was removed under reduced vacuum, the residue diluted with H₂O and CH₂Cl₂, and the solution made basic with 1N NaOH. The layers were separated and the aqueous layer further extracted with CH₂Cl₂ (2×30 mL). The combined organic layers were dried (Na₂SO₄), filtered, and concentrated to afford the 2-chlorobenzimidazole (0.191 g, 80%) as a tan solid. ¹H NMR (CDCl₃): [symbol] 7.76 (dd, J=0.8, 8.0 Hz, 1H), 7.65 (dd, J=0.8, 1.6 Hz, 1H), 7.55 (dd, J=1.6, 8.0 Hz, 1H), 3.85 (s, 3H).

(c) 1-Methyl-2-{[2-(4-morpholinyl)ethyl]amino}-1H-benzimidazole-6-carbonitrile. A solution of 2-chloro-1-methyl-1H-benzimidazole-6-carbonitrile (50 mg, 0.262 mmol) and 4-(2-aminoethyl)morpholine (136 mg, 1.05 mmol) in EtOH (1.3 mL) was heated in a sealed vial to 80° C. for 24 h. The cooled reaction mixture was concentrated and the crude residue purified by column chromatography to yield the 2-aminobenzimidazole (66 mg, 88%). ¹H NMR (CDCl₃): [symbol] 7.45 (d, J=8.4 Hz, 1H), 7.39 (dd, J=1.2, 8.4 Hz, 1H), 7.32 (d, J=0.8 Hz, 1H), 5.25 (m, 1H), 3.74 (t, J=4.8 Hz, 4H), 3.64 (m, 2H), 3.53 (s, 3H), 2.70 (t, J=5.6 Hz, 2H), 2.53 (t, J=4.4 Hz, 4H).

(d) 1-Methyl-2-{[2-(4-morpholinyl)ethyl]amino}-1H-benzimidazole-6-carbaldehyde. Following the procedure of Example 9(b), the benzimidazole-6-carbonitrile from Example 41(c) was reduced to the aldehyde in 73% yield. ¹H NMR (CDCl₃): [symbol] 9.56 (s, 1H), 7.63 (m, 2H), 7.50 (d, J=8.0 Hz, 1H), 5.27 (bs, 1H), 3.74 (t, J=4.8 Hz, 4H), 3.66 (app. quartet, J=4.8, 11.2 Hz, 2H), 3.56 (s, 3H), 2.71 (t, J=5.6 Hz, 2H), 2.54 (t, J=4.4 Hz, 4H).

(e) (5Z)-2-[(2-Chlorophenyl)amino]-5-[(1-methyl-2-{[2-(4-morpholinyl)ethyl]amino}-1H-benzimidazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 41 | (5Z)-2-[(2-Chloro- | (d₆-acetone): 7.76 (s, 1H), 7.51 (m, |
| | phenyl)- | 1H), 7.37 (t, J = 7.2 Hz, 1H), 7.30 |
| | amino]-5-[(1-methyl- | (m, 2H), 7.21 (m, 2H), 7.16 (m, |
| | 2-{[2-(4-morpholinyl)- | 1H), 3.60 (m, 10H), 2.64 (m, 2H), |
| | ethyl]-amino}-1H- | 2.49 (bs, 4H) |
| | benzimidazol- | |
| | 6-yl)methylidene]-1,3- | |
| | thiazol-4(5H)-one | |

### EXAMPLES 42-45

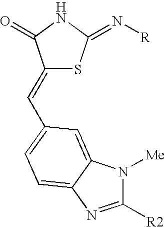

The following compounds were prepared according to the procedure of Example 41, using the requisite amine for 4-(2-aminoethyl)morpholine and substituting the appropriate thiazolone for 2-[(2-chlorophenyl)amino]-1,3-thiazol-4(5H)-one:

| | Compound | | | |
|---|---|---|---|---|
| Example | name | R2 | R | NMR (400 MHz) |
| 42 | (5Z)-2-[(2,6-dichlorophenyl)amino]-5-[(1-methyl-2-{[2-(4-morpholinyl)ethyl]amino}-1H-benzimidazol-6-yl)methylidene]-1,3-thiazol-4(5H)-one | 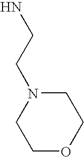 | 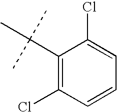 | (CD₃OD): 7.67 (s, 1 H), 7.41 (d,J = 8.4 Hz, 2 H), 7.28 (m, 1 H),7.19 (m, 2 H), 7.11 (m, 1 H), 3.71(m, 4 H), 3.59 (m, 2 H), 3.50 (s,3 H), 3.08 (t, J = 7.2 Hz, 2 H),2.68 (m, 2 H), 2.55 (m, 4 H) |
| 43 | (5Z)-2-[(2,4-difluorophenyl)amino]-5-[(1-methyl-2-{[2-(4-morpholinyl)ethyl]amino}-1H-benzimidazol-6-yl)methylidene]-1,3-thiazol-4(5H)-one | 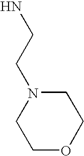 | 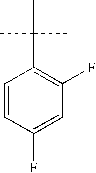 | (CD₃OD): 7.74 (s, 1 H), 7.37 (m,1 H), 7.29 (m, 1 H), 7.22 (m, 1 H),7.06 (m, 3 H), 3.70 (bs, 4 H), 3.59(m, 2 H), 3.52 (s, 3 H), 2.68 (m,2 H), 2.56 (bs, 4 H). |
| 44 | (5Z)-2-[(2-chlorophenyl)amino]-5-[(2-{[2-(dimethylamino)ethyl]amino}-1-methyl-1H-benzimidazol-6-yl)methylidene]-1,3-thiazol-4(5H)-one | 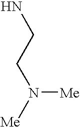 | 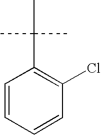 | (CD₃OD): 7.71 (s, 1 H), 7.47 (d,J = 7.6 Hz, 1 H), 7.32 (m, 2 H),7.17 (m, 3 H), 7.10 (m, 1 H), 3.61(t, J = 6.0 Hz, 2 H), 3.51 (s, 3 H),2.73 (t, J = 6.4 Hz, 2 H), 2.39 (s,6 H) |
| 45 | (5Z)-2-[(2-chlorophenyl)amino]-5-({2-[(2-hydroxyethyl)amino]-1-methyl-1H-benzimidazol-6-yl}methylidene)-1,3-thiazol-4(5H)-one | 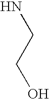 | 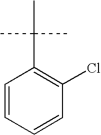 | (CD₃OD): 7.76 (s, 1 H), 7.48 (d,J = 8.0 Hz, 1 H), 7.33 (t, J = 7.6Hz, 1 H), 7.28 (dd, J = 0.8, 7.2Hz, 1 H), 7.20 (m, 3 H), 7.09 (m,1 H), 3.76 (t, J = 5.2 Hz, 2 H),3.56 (t, J = 5.2 Hz, 2 H), 3.51 (s,3 H) |

### EXAMPLE 46

### (5Z)-2-[(2-Chlorophenyl)amino]-5-{[1-methyl-2-(4-morpholinylmethyl)-1H-benzimidazol-6-yl]methylidene}-1,3-thiazol-4(5H)-one

(a) 2-(Chloromethyl)-1-methyl-1H-benzimidazole-6-carbonitrile. To a mixture of 4-amino-3-(methylamino)benzonitrile (0.20 g, 1.36 mmol) in EtOH (6.8 mL) was added ethyl 2-chloroethanimidoate hydrochloride (0.43 g, 2.72 mmol; prepared according to Stillings et al. in J. Med. Chem. 1986, 29, 2280-2284). The mixture was stirred overnight and the solvent removed. The residue was diluted with H₂O and extracted with CH₂Cl₂ (3×20 mL). The combined organic layers were dried (Na₂SO₄), filtered and concentrated under reduced vacuum to afford the crude benzimidazole (0.266 g, 95%). ¹H NMR (CDCl₃): [symbol] 7.82 (d, J=8.4 Hz, 1H), 7.72 (s, 1H), 7.56 (dd, J=1.2, 8.4 Hz, 1H), 4.86 (s, 2H), 3.93 (s, 3H).

(b) 1-Methyl-2-(4-morpholinylmethyl)-1H-benzimidazole-6-carbonitrile. To a mixture of 2-(chloromethyl)-1-methyl-1H-benzimidazole-6-carbonitrile (60 mg, 0.293 mmol) in EtOH (1 mL) was added morpholine (0.1 mL, 1.17 mmol). The mixture was heated to reflux for 1 h, cooled and concentrated to dryness. The residue was taken up in CH₂Cl₂ and treated with sat. aq. NaHCO₃. The layers were separated and the aqueous layer further extracted with CH₂Cl₂ (2×). The combined organic layers were dried over Na₂SO₄, filtered and the solvent removed in vacuo to provide the 2-methylmorpholino benzimidazole (73 mg, 100%). ¹H NMR (CDCl₃): [symbol] 7.78 (d, J=8.4 Hz, 1H), 7.69 (s, 1H), 7.52 (dd, J=1.2, 8.4 Hz, 1H), 3.93 (s, 3H), 3.85 (s, 2H), 3.70 (t, J=4.8H, 4H), 2.54 (t, J=4.8 Hz, 4H).

(c) 1-Methyl-2-(4-morpholinylmethyl)-1H-benzimidazole-6-carbaldehyde. Following the procedure of Example 9(b), the benzimidazole-6-carbonitrile from Example 46(b) was reduced to the aldehyde in 100% yield. ¹H NMR (CDCl₃): [symbol] 10.10 (s, 1H), 7.95 (s, 1H), 7.83 (d, J=8.0 Hz, 1H), 7.79 (dd, J=1.2, 8.0 Hz, 1H), 3.97 (s, 3H), 3.86 (s, 2H), 3.71 (t, J=4.8 Hz, 4H), 2.55 (t, J=4.8 Hz, 4H).

(d) (5Z)-2-[(2-Chlorophenyl)amino]-5-{[1-methyl-2-(4-morpholinylmethyl)-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 46 | (5Z)-2-[(2-Chloro- | (CD₃OD): 7.81 (s, 1H), 7.62 (m, 2H), |
| | phenyl)- | 7.47 (dd, J = 1.2, 8.0 Hz, 1H), 7.38 (d, J = 8.4 Hz, |
| | amino]-5-[(1-methyl- | 1H), 7.32 (dt, J = 1.2, 7.6 Hz, |
| | 2-{[2-(4-morpholinyl)ethyl]- | 1H), 7.18 (dt, J = 1.2, 8.0 Hz, 1H), |
| | amino}-1H-benzimidazol-6- | 7.09 (dd, J = 0.8, 7.6 Hz, 1H), 3.91 (s, 3H), |
| | yl)methylidene]-1,3- | 3.83 (s, 2H), 3.66 (bs, 4H), 2.50 (bs, |
| | thiazol-4(5H)-one | 4H) |

### EXAMPLES 47-49

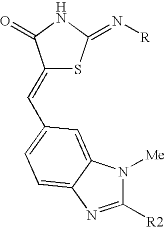

The following compounds were prepared according to the procedure of Example 46, using the requisite amine nucleophile and substituting the appropriate thiazolone for 2-[(2-chlorophenyl)amino]-1,3-thiazol-4(5H)-one:

| Example | Compound name | R2 | R | NMR (400 MHz) |
|---|---|---|---|---|
| 47 | (5Z)-2-[(2,6-dichlorophenyl)amino]-5-{[1-methyl-2-(4-morpholinylmethyl)-1H-benzimidazol-6-yl]methylidene}-1,3-thiazol-4(5H)-one | 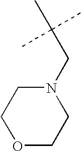 | 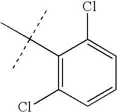 | (CD₃OD): 7.68 (m, 1 H), 7.60(m, 2 H), 7.40 (m, 3 H), 7.09 (m,1 H), 3.90 (s, 3 H), 3.82 (s, 2 H),3.67 (bs, 4 H), 2.50 (bs, 4 H) |
| 48 | (5Z)-2-[(2-chlorophenyl)amino]-5-({1-methyl-2-[(4-methyl-1-piperazinyl)methyl]-1H-benzimidazol-6-yl}methylidene)-1,3-thiazol-4(5H)-one | 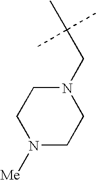 | 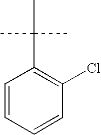 | (CD₃OD): 7.90 (m, 1 H), 7.87 (s,1 H), 7.80 (m, 1 H), 7.64 (m, 1 H),7.47 (d, J = 7.6 Hz, 1 H), 7.30 (t,J = 6.8 Hz, 1 H), 7.19 (t, J = 7.2Hz, 1 H), 7.08 (m, 1 H), 4.17 (m,2 H), 3.99 (s, 3 H), 3.50 (m, 2 H),3.12 (m, 4 H), 2.92 (s, 3 H), 2.70(m, 2 H) |
| 49 | (5Z)-2-[(2,6-dichlorophenyl)amino]-5-({1-methyl-2-[(4-methyl-1-piperazinyl)methyl]-1H-benzimidazol-6-yl}methylidene)-1,3-thiazol-4(5H)-one | 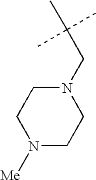 | 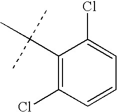 | (CD₃OD): 7.90 (s, 1 H), 7.89 (s,1 H), 7.78 (d, J = 8.4 Hz, 1 H),7.57 (t, J = 7.6 Hz, 1 H), 7.44 (d,J = 8.0 Hz, 2 H), 7.15 (t, J = 8.4Hz, 1 H), 4.14 (s, 2 H), 3.98 (s,3 H), 3.50 (m, 2 H), 3.12 (m, 4 H),2.91 (s, 3 H), 2.68 (m, 2 H) |

### EXAMPLE 50

### (5Z)-2-[(2-Chlorophenyl)amino]-5-{[1-methyl-2-(trifluoromethyl)-1H-benzimidazol-6-yl]methylidene}-1,3-thiazol-4(5H)-one

(a) 1-Methyl-2-(trifluoromethyl)-1H-benzimidazole-6-carbonitrile. 4-Amino-3-(methylamino)benzonitrile (80 mg, 0.544 mmol) and trifluoroacetic acid (1.1 mL) were heated to reflux for 6 h. Upon cooling, the excess TFA was removed under reduced pressure and sat. aq. NaHCO₃ carefully added. A beige solid precipitated, which was filtered, rinsed with H₂O and dried to afford the 2-trifluoromethyl benzimidazole (100 mg, 82%). ¹H NMR (CDCl₃): [symbol] 7.98 (d, J=8.0 Hz, 1H), 7.84 (s, 1H), 7.65 (dd, J=1.2, 8.0 Hz, 1H), 4.01 (s, 3H).

(b) 1-Methyl-2-(trifluoromethyl)-1H-benzimidazole-6-carbaldehyde. Following the procedure of Example 9(b), the benzimidazole-6-carbonitrile from Example 50(a) was reduced to the aldehyde in 70% yield. ¹H NMR (CDCl₃): [symbol] 10.15 (s, 1H), 8.06 (s, 1H), 8.01 (d, J=8.4 Hz, 1H), 7.92 (d, J=8.0 Hz, 1H), 4.04 (s, 3H).

(c) (5Z)-2-[(2-Chlorophenyl)amino]-5-{[1-methyl-2-(trifluoromethyl)-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 50 | (5Z)-2-[(2-Chlorophenyl)- | (CD₃OD): 7.88 (s, 1H), 7.82 |
| | amino]-5-{[1-methyl-2- | (m, 2H), 7.52 (d, J = 8.8 Hz, |
| | (trifluoromethyl)- | 1H), 7.48 (d, J = 8.0 Hz, 1H), |
| | 1H-benzimidazol-6-yl]- | 7.33 (t, J = 7.6 Hz, 1H), |
| | methylidene}-1,3- | 7.19 (t, J = 7.6 Hz, 1H), 7.10 (d, |
| | thiazol-4(5H)-one | J = 7.6 Hz, 1H), 3.99 (s, 3H) |

### EXAMPLES 51-52

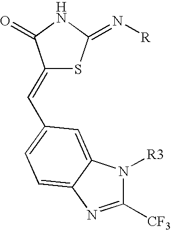

The following compounds were prepared according to the procedure of Example 50, using the requisite diaminobenzonitrile starting material and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 51 | (5Z)-2-[(2,6-dichlorophenyl)amino]-5-{[1-methyl-2-(trifluoromethyl)-1H-benzimidazol-6-yl]methylidene}-1,3-thiazol-4(5H)-one | Me | 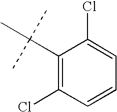 | (CD₃OD): 7.90 (s1 H), 7.81 (m, 2 H),7.50 (dd, J = 0.8, 8.4Hz, 1 H), 7.44 (d, J =8.4 Hz, 2 H), 7.15 (d,J = 8.0 Hz, 1 H), 4.00(s, 3 H) |
| 52 | (5Z)-2-[(2,6-dichlorophenyl)amino]-5-{[1-[2-(dimethylamino)ethyl]-2-(trifluoromethyl)-1H-benzimidazol-6-yl]methylidene}-1,3-thiazol-4(5H)-one | 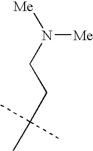 | 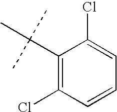 | (CD₃OD): 7.90 (m,3 H), 7.55 (d, J = 8.0Hz, 1 H), 7.46 (d, J =8.0 Hz, 2 H), 7.17 (t, J =8.0 Hz, 1 H), 3.59(m, 2 H), 3.06 (m,2 H), 3.02 (s, 6 H) |

### EXAMPLE 53

### (5Z)-2-[(2-Chlorophenyl)amino]-5-{[2-(1,1-dimethylethyl)-1-methyl-1H-benzimidazol-6-yl]methylidene}-1,3-thiazol-4(5H)-one

(a) 2-(1,1-Dimethylethyl)-1-methyl-1H-benzimidazole-6-carbonitrile. To a mixture of Cu(OAc)₂ (800 mg, 4.76 mmol) in AcOH (3.6 mL) and H₂O (1.2 mL), heated to 55° C., was added 4-amino-3-(methylamino)benzonitrile (70 mg, 0.48 mmol) and pivaldehyde (57 □L, 0.52 mmol). Heating was continued for 2 h, then the solvent was removed under reduced pressure. EtOAc (50 mL) was added, which was washed with sat. aq. NaHCO₃. The layers were separated, the aqueous layer extracted with EtOAc (20 mL), and the combined organic layer washed with brine (2×20 mL), dried (Na₂SO₄), filtered and concentrated to afford the 2-t-butyl benzimidazole (86 mg, 85%). ¹H NMR (CDCl₃): [symbol] 7.78 (d, J=8.4 Hz, 1H), 7.62 (d, J=1.2 Hz, 1H), 7.50 (dd, J=1.2, 8.4 Hz, 1H), 3.95 (s, 3H), 1.58 (s, 9H).

(b) 2-(1,1-Dimethylethyl)-1-methyl-1H-benzimidazole-6-carbaldehyde. Following the procedure of Example 9(b), the benzimidazole-6-carbonitrile from Example 53(a) was reduced to the aldehyde in 93% yield. ¹H NMR (CDCl₃): [symbol] 10.08 (s, 1H), 7.89 (s, 1H), 7.84 (d, J=8.4 Hz, 1H), 7.77 (dd, J=1.2, 8.4 Hz, 1H), 3.99 (s, 3H), 1.59 (s, 9H).

(c) (5Z)-2-[(2-Chlorophenyl)amino]-5-{[2-(1,1-dimethylethyl)-1-methyl-1H-benzimidazol-6-yl]methylidene}-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 53 | (5Z)-2-[(2- | (CD₃OD): 7.97 (s, 1H), 7.87 (s, 1H), 7.80 (d, J = 8.8 Hz, |
| | chlorophenyl)amino]-5-{[2- | 1H), 7.70 (d, J = 8.4 Hz, 1H), 7.47 (d, J = 8.0 Hz, |
| | (1,1-dimethylethyl)-1- | 1H), 7.32 (dt, J = 0.8, 7.6 Hz, 1H), |
| | methyl-1H-benzimidazol-6- | 7.19 (dt, J = 1.2, 8.0 Hz, 1H), 7.08 (dd, J = 1.2, 7.6 Hz, |
| | yl]methylidene}-1,3-thiazol- | 1H), 4.17 (s, 3H), 1.66 (s, 9H) |
| | 4(5H)-one | |

### EXAMPLE 54

### (5Z)-2-[(2,6-Dichlorophenyl)amino]-5-{[2-(1,1-dimethylethyl)-1-methyl-1H-benzimidazol-6-yl]methylidene}-1,3-thiazol-4(5H)-one

The title compound was prepared according to the procedure of Example 53, except substituting 2-[(2,6-dichlorophenyl)amino]-1,3-thiazol-4(5H)-one for 2-[(2-chlorophenyl)amino]-1,3-thiazol-4(5H)-one:

| Example | Compound name | NMR (400 MHz) |
|---|---|---|
| 54 | (5Z)-2-[(2,6- | (CD₃OD): 7.94 (s, 1H), 7.91 (s, 1H), 7.81 (d, J = 8.4 Hz, |
| | dichlorophenyl)amino]-5- | 1H), 7.68 (d, J = 8.4 Hz, 1H), 7.44 (d, J = 8.4 Hz, |
| | {[2-(1,1-dimethylethyl)-1- | 2H), 7.15 (t, J = 8.4 Hz, 1H), 4.18 (s, 3H), |
| | methyl-1H-benzimidazol-6- | 1.66 (s, 9H) |
| | yl]methylidene}-1,3-thiazol- | |
| | 4(5H)-one | |

### EXAMPLE 55

### (5Z)-2-[(2-Chlorophenyl)amino]-5-[(1-methyl-1H-1,2,3-benzotriazol-6-yl)methylidene]-1,3-thiazol-4(5H)-one

(a) 1-Methyl-1H-1,2,3-benzotriazole-6-carbonitrile. To a 0° C. mixture of 4-amino-3-(methylamino)benzonitrile (60 mg, 0.408 mmol) in conc. HCl (0.55 mL) was added NaNO₂ (31 mg, 0.449 mmol) in H₂O (0.2 mL). The mixture was allowed to warm to room temperature and stirred for 1 h. After recooling to 0° C., the mixture was treated with 6N NaOH until basic, the precipitate filtered, rinsed with H₂O and dried to afford the benzotriazole nitrile (50 mg, 77%). ¹H NMR (CDCl₃): [symbol] 8.19 (d, J=8.4 Hz, 1H), 7.95 (s, 1H), 7.62 (d, J=8.4 Hz, 1H), 4.38 (s, 3H).

(b) 1-Methyl-1H-1,2,3-benzotriazole-6-carbaldehyde. Following the procedure of Example 9(b), the benzimidazole-6-carbonitrile from Example 55(a) was reduced to the aldehyde in 92% yield. ¹H NMR (CDCl₃): [symbol] 10.20 (s, 1H), 8.20 (d, J=8.8 Hz, 1H), 8.11 (t, J=1.2 Hz, 1H), 7.93 (dd, J=1.2, 8.8 Hz, 1H), 4.41 (s, 3H).

(c) (5Z)-2-[(2-Chlorophenyl)amino]-5-[(1-methyl-1H-1,2,3-benzotriazol-6-yl)methylidene]-1,3-thiazol-4(5H)-one. Knoevenagel coupling with 2-[(2-chlorophenyl)amino]-1,3-thiazol-4(5H)-one was accomplished using the procedure of Example 1(e).

| Example | Compound name | NMR (400 MHz) |
|---|---|---|
| 55 | (5Z)-2-[(2- | (CD₃OD): 8.01 (d, J = 9.2 Hz, |
| | Chlorophenyl)amino]- | 1H), 7.87 (s, 2H), 7.53 (d, J = 9.6 |
| | 5- | Hz, 1H), 7.48 (d, J = 7.6 Hz, |
| | [(1-methyl-1H-1,2,3- | 1H), 7.33 (dt, J = 0.8, 7.2 Hz, |
| | benzotriazol-6- | 1H), 7.19 (m, 1H), 7.09 (d, |
| | yl)methylidene]- | J = 8.0 Hz, 1H), 4.32 (s, 3H) |
| | 1,3-thiazol-4(5H)-one | |

### EXAMPLES 56-58

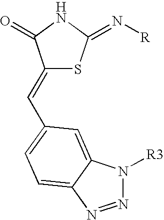

The following compounds were prepared according to the procedure of Example 55, using the requisite diaminobenzonitrile starting material and substituting the appropriate thiazolone for 2-[(2-chlorophenyl)amino]-1,3-thiazol-4(5H)-one:

| Example | Compound name | R3 | R | NMR (400 MHz) |
|---|---|---|---|---|
| 56 | (5Z)-2-[(2,6-dichlorophenyl)amino]-5-[(1-methyl-1H-1,2,3-benzotriazol-6-yl)methylidene]-1,3-thiazol-4(5H)-one | Me | 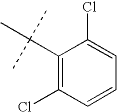 | (CD₃OD): 8.01 (d, J = 8.8 Hz,1 H), 7.91 (s, 1 H), 7.87 (s, 1 H),7.52 (d, J = 8.8 Hz, 1 H), 7.44 (d,J = 8.0 Hz, 2 H), 7.16 (t, J = 8.0Hz, 1 H), 4.32 (s, 3 H) |
| 57 | (5Z)-2-[(2-chlorophenyl)amino]-5-({1-[2-(dimethylamino)ethyl]-1H-1,2,3-benzotriazol-6-yl}methylidene)-1,3-thiazol-4(5H)-one | 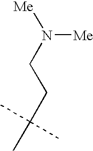 | 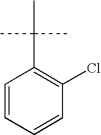 | (CD₃OD): 8.10 (d, J = 8.8 Hz,1 H), 7.96 (s, 1 H), 7.89 (s, 1 H),7.60 (d, J = 10.0 Hz, 1 H), 7.48(d, J = 8.0 Hz, 1 H), 7.33 (t, J =7.6 Hz, 1 H), 7.19 (dt, J = 1.2,7.6 Hz, 1 H), 7.08 (d, J = 8.0 Hz,1 H), 5.15 (t, J = 6.0 Hz, 2 H),3.87 (t, J = 5.6 Hz, 2 H), 3.01 (s,6 H) |
| 58 | (5Z)-2-[(2,6-dichlorophenyl)amino]-5-({1-[2-(dimethylamino)ethyl]-1H-1,2,3-benzotriazol-6-yl}methylidene)-1,3-thiazol-4(5H)-one | 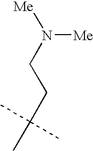 | 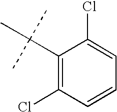 | (CD₃OD): 8.10 (d, J = 8.4 Hz,1 H), 7.97 (s, 1 H), 7.91 (s, 1 H),7.57 (d, J = 8.4 Hz, 1 H), 7.44 (d,J = 8.0 Hz, 2 H), 7.16 (t, J = 8.0Hz, 1 H), 5.16 (t, J = 6.0 Hz, 2 H),3.88 (t, J = 6.0 Hz, 2 H), 3.01 (s,6 H) |

### EXAMPLE 59

### 2-(2,6-Dichloro-phenylimino)-5-(2-methyl-benzooxazol-6-yl-methylene)-thiazolidin-4-one

### a) 2-Methyl-benzooxazole-6-carboxylic acid methyl ester

A suspension of methyl 4-amino-3-hydroxy-benzoate (30 g, 0.18 mol) in triethylorthoacetate (90 mL) was heated to 100° C. for 3 hours. Ethanol (150 mL) was added followed by water (50 mL). The reaction mixture was filtered to yield 25 g (72% yield) of pure 2-methyl-benzooxazole-6-carboxylic acid methyl ester. ¹H-NMR (CDCl₃): [symbol] 2.67 (s, 3H), 3.94 (s, 3H), 7.65 (d, 1H, J=8.1 Hz), 8.02 (dd, 1H, J=8.1 Hz, J′=1.5 Hz), 8.15 (d, 1H, J=1.5 Hz). LC MS (m/e)=192.2 (MH+). Rt=1.70 min

### b) (2-Methyl-benzooxazol-6-yl)-methanol

To the solution of 2-methyl-benzooxazole-6-carboxylic acid methyl ester (25 g, 0.13 mol) in THF (500 mL) at −20° C. was added a solution of lithium aluminum hydride (4.81 g, 130 mL of 1 M solution in THF, 0.13 mmol, 1 eq) and the reaction mixture was allowed to warm up to the room temperature overnight. Water (5 mL) followed by 1 M NaOH solution (5 mL) followed by water (15 mL) was added and the reaction mixture was stirred for 15 min at the room temperature. The suspension was filtered, liquid evaporated and purified by column chromatography (1:3 ethyl acetate:dichloromethane) to give 12.5 g (58% yield) of pure (2-methyl-benzooxazol-6-yl)-methanol. ¹H-NMR (CDCl₃): [symbol] 2.64 (s, 3H), 4.82 (s, 2H), 7.29 (d, 1H, J=8 Hz), 7.53 (s, 1H), 7.62 (d, 1H, J=8 Hz). LC MS (m/e)=164.2 (MH+). Rt=1.03 min.

### c) 2-Methyl-benzooxazole-6-carbaldehyde

To the solution (2-methyl-benzooxazol-6-yl)-methanol (12.5 g, 76 mmol) in dichloromethane (200 mL) was added pyridinium chlorochromate (20 g, 93 mmol, 1.2 eq) and the reaction mixture was stirred for 1 hour at the room temperature. Celite (10 g) was added followed by decolorizing carbon (2 g) and the reaction mixture was filtered after 15 min of stirring. After evaporation the crude product was purified column chromatography (1:10 ethyl acetate:dichloromethane) of give 8.2 g (66% yield) of pure 2-methyl-benzooxazole-6-carbaldehyde. ¹H-NMR (CDCl₃): [symbol] 2.73 (s, 3H), 7.79 (d, 1H, J=8.1 Hz), 7.88 (dd, 1H, J=8.1 Hz, J′=1.2 Hz), 8.03 (d, 1H, J=1.2 Hz), 10.09 (s, 1H). LC MS (m/e)=162.2 (MH+). Rt=1.47 min.

### d) 2-(2,6-Dichloro-phenylimino)-5-(2-methyl-benzooxazol-6-yl-methylene)-thiazolidin-4-one

To the solution of 2-(2,6 dichloro-phenylimino)-thiazolidin-4-one (483 mg, 1.85 mmol) in acetic acid (8 mL) was added 2-methyl-benzooxazole-6-carbaldehyde (300 mg, 1.85 mmol, 1 eq) followed by sodium acetate (0.8 g). The reaction mixture was refluxed for 48 hours and water (10 mL) was added. Solid was filtered and purified by column chromatography (1:5 ethyl acetate:dichloromethane) to give 110 mg (15% yield) of pure 2-(2,6-dichloro-phenylimino)-5-(2-methyl-benzooxazol-6-yl-methylene)-thiazolidin-4-one. ¹H-NMR (CDCl₃): [symbol] 2.69 (s, 3H), 7.12 (t, 1H, J=8.1 Hz), 7.36 (d, 3H, J=7.8 Hz), 7.56 (s, 1H), 7.70 (d, 1H, J=8.1 Hz), 7.88 (s, 1H), 9.69 (s, 1H). LC MS (m/e)=404.0 (MH+). Rt=2.36 min.

### EXAMPLE 60

### 2-(2,6-Difluoro-phenylimino)-5-(2-methyl-benzooxazol-6-yl-methylene)-thiazolidin-4-one

Following the procedure of example 59 (d), starting from 2-(2,6-difluoro-phenylimino)-thiazolidin-4-one, the title compound was prepared as a yellow solid (82 mg, 22%). ¹H-NMR (CDCl₃): [symbol] 2.69 (s, 3H), 7.30 (t, 2H, J=7.9 Hz), 7.15 (m, 1H), 7.41 (d, 1H, J=8.3 Hz), 7.57 (s, 1H), 7.70 (d, 1H, J=8.1 Hz), 7.81 (s, 1H), LC MS (m/e)=372.0 (MH+). Rt=2.13 min.

### EXAMPLE 61

### 2-(2-Fluoro-phenylimino)-5-(2-methyl-benzooxazol-6-yl-methylene)-thiazolidin-4-one

To the solution of 2-(2,6-difluoro-phenylimino)-thiazolidin-4-one (105 mg, 0.5 mmol) in ethanol (5 mL) was added 2-methyl-benzooxazole-6-carbaldehyde (80 mg, 0.5 mmol, 1 eq) followed by piperidine (0.1 mL). The reaction mixture was refluxed for 48 hours and diethyl ether (3 mL) was added. Solid was filtered to give 58 mg (33% yield) of pure 2-(2-fluoro-phenylimino)-5-(2-methyl-benzooxazol-6-yl-methylene)-thiazolidin-4-one. LC MS (m/e)=354.2 (MH+). Rt=2.11 min.

### EXAMPLES 62-71

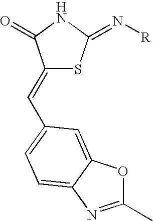

The following compounds were prepared according to the procedure of Example 61, except substituting the appropriately substituted thiazolidinone for 2-(2,6-difluoro-phenylimino)-thiazolidin-4-one.

| | | Thiazolidinone | LC MS | |
|---|---|---|---|---|
| Example | Product name | substituted | (m/e) | Rt (min) |
| 62 | 2-(2-Chloro-phenylimino)-5-(2-methyl-benzooxazol-6-ylmethylene)-thiazolidin-4-one | 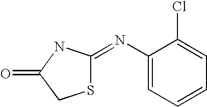 | 370.0 | 2.23 |
| 63 | 2-(2-Trifluromethyl-phenylimino)-5-(2-methyl-benzooxazol-6-ylmethylene)-thiazolidin-4-one | 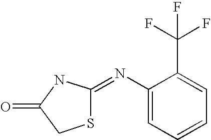 | 404.0 | 2.34 |
| 64 | 2-(2,4-Difluoro-phenylimino)-5-(2-methyl-benzooxazol-6-ylmethylene)-thiazolidin-4-one | 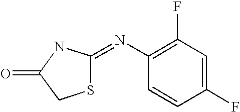 | 372.0 | 2.16 |
| 65 | 2-(2,5-Dichloro-phenylimino)-5-(2-methyl-benzooxazol-6-ylmethylene)-thiazolidin-4-one | 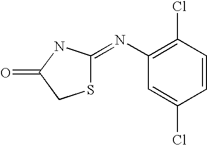 | 404.2 | 2.46 |
| 66 | 2-(2,4-Dimethyl-phenylimino)-5-(2-methyl-benzooxazol-6-ylmethylene)-thiazolidin-4-one | 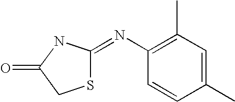 | 364.2 | 2.31 |
| 67 | 2-(4-Cyano-phenylimino)-5-(2-methyl-benzooxazol-6-ylmethylene)-thiazolidin-4-one | 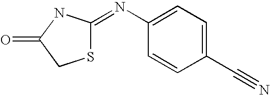 | 361.0 | 2.07 |
| 68 | 4-[5-(2-Methyl-benzooxazol-6-ylmethylene)-4-oxo-thiazolidin-2-ylideneamino]-benzoicacid | 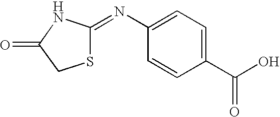 | 380.0 | 1.99 |
| 69 | 2-(2,4-Dichloro-phenylimino)-5-(2-methyl-benzooxazol-6-ylmethylene)-thiazolidin-4-one | 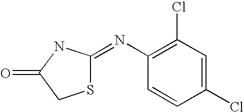 | 404.0 | 2.52 |
| 70 | 2-(2,5-Difluoro-phenylimino)-5-(2-methyl-benzooxazol-6-ylmethylene)-thiazolidin-4-one | 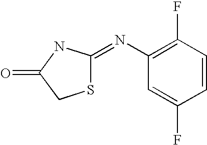 | 372.0 | 2.20 |
| 71 | 5-(2-Methyl-benzooxazol-6-ylmethylene)-2-phenylimino-thiazolidin-4-one | 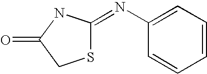 | 336.2 | 2.11 |

### EXAMPLE 72

### 5-(2-Methyl-benzooxazol-6-ylmethylene)-2-(2-piperidin-1-yl-ethylimino)-thiazolidin-4-one

### a) 5-(2-Methyl-benzooxazol-6-ylmethylene)-2-thioxo-thiazolidin-4-one

To the solution of rhodanine (1.21 g, 10 mmol) in ethanol (50 mL) was added 2-methyl-benzooxazole-6-carbaldehyde (1.61 mg, 10 mmol, 1 eq) followed by pyridine (1 mL). The reaction mixture was refluxed for 24 hours cooled to the room temperature. Solid was filtered to give 1.3 g (47% yield) of pure 5-(2-methyl-benzooxazol-6-ylmethylene)-2-thioxo-thiazolidin-4-one. ¹H-NMR (DMSO): [symbol] 2.67 (s, 3H), 2.85 (s, 3H), 7.66 (dd, 1H, J=8.3 Hz, J′=1.7 Hz), 7.82 (d, 1H, J=8.3 Hz), 8.00 (s, 1H), 8.02 (d, 1H, J=1.7 Hz). LC MS (m/e)=277.0 (MH+). Rt=2.02 min.

### b) 5-(2-Methyl-benzooxazol-6-ylmethylene)-2-methylsulfanyl-thiazol-4-one

To the solution of 5-(2-methyl-benzooxazol-6-ylmethylene)-2-thioxo-thiazolidin-4-one (200 mg, 0.72 mmol) in ethanol (5 mL) was added diisopropyl ethyl amine (0.185 mL, 1.44 mmol, 2 eq) followed by iodomethane (0.216 mL, 3.5 mmol, 5 eq). The reaction mixture was stirred overnight, then filtered. Solid was washed with cold ethanol to give 193 mg (92% yield) of pure 5-(2-methyl-benzooxazol-6-ylmethylene)-2-methylsulfanyl-thiazol-4-one. ¹H-NMR (DMSO): [symbol] 2.67 (s, 3H), 7.59 (dd, 1H, J=8.3 Hz, J′=1.5 Hz), 7.80 (s, 1H), 7.82 (d, 1H, J=8.3 Hz), 7.96 (d, 1H, J=1.5 Hz). LC MS (m/e)=291.0 (MH+). Rt=2.41 min.

### c) 5-(2-Methyl-benzooxazol-6-ylmethylene)-2-(2-piperidin-1-yl-ethylimino)-thiazolidin-4-one

To the solution of 5-(2-methyl-benzooxazol-6-ylmethylene)-2-methylsulfanyl-thiazol-4-one (40 mg, 0.14 mmol) in ethanol (3 mL) was added 2-piperidin-1-yl-ethylamine (25 mg, 0.2 mmol, 1.4 eq) and the reaction mixture was heated under reflux for 24 hours. Diethyl ether (3 mL) was added and product isolated by filtration to give 27 mg (53% yield) of pure 5-(2-methyl-benzooxazol-6-ylmethylene)-2-(2-piperidin-1-yl-ethylimino)-thiazolidin-4-one. LC MS (m/e)=371.0 (MH+). Rt=1.40 min.

### EXAMPLES 73-85

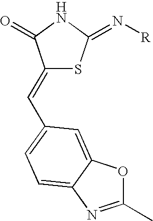

The following compounds were prepared according to the procedure of Example 72 (c), except substituting the appropriate amine listed below for 2-piperidin-1-yl-ethylamine.

| | | | LC MS | |
|---|---|---|---|---|
| Example | Product name | Amine used | (m/e) | Rt (min) |
| 73 | 2-(2-Methoxy-ethylimino)-5-(2-methyl-benzooxazol-6-ylmethylene)-thiazolidin-4-one | 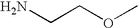 | 318.0 | 1.52 |
| 74 | 5-(2-Methyl-benzooxazol-6-ylmethylene)-2-(3-morpholin-4-yl-propylimino)-thiazolidin-4-one | 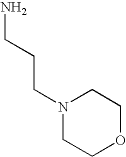 | 387.2 | 1.31 |
| 75 | 3-[5-(2-Methyl-benzooxazol-6-ylmethylene)-4-oxo-thiazolidin-2-ylideneamino]-benzenesulfonamide | 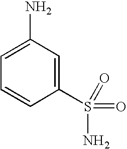 | 415.2 | 1.68 |
| 76 | 2-(4-Hydroxy-butylimino)-5-(2-methyl-benzooxazol-6-ylmethylene)-thiazolidin-4-one | 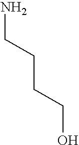 | 332.2 | 1.49 |
| 77 | 2-(trans-4-Hydroxy-cyclohexylimino)-5-(2-methyl-benzooxazol-6-ylmethylene)-thiazolidin-4-one | 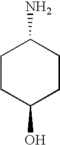 | 358.0 | 1.45 |
| 78 | 5-(2-Methyl-benzooxazol-6-ylmethylene)-2-phenethylimino-thiazolidin-4-one | 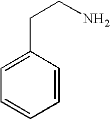 | 363.8 | 2.00 |
| 79 | 4-{2-[5-(2-Methyl-benzooxazol-6-ylmethylene)-4-oxo-thiazolidin-2-ylideneamino]-ethyl}-benzenesulfonamide | 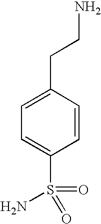 | 443.2 | 1.63 |
| 80 | 2-(2-Benzo[1,3]dioxol-5-yl-ethylimino)-5-(2-methyl-benzooxazol-6-ylmethylene)-thiazolidin-4-one | 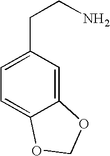 | 408.2 | 1.97 |
| 81 | 2-(4-Chloro-phenylimino)-5-(2-methyl-benzooxazol-6-ylmethylene)-thiazolidin-4-one | 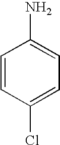 | 370.0 | 2.31 |
| 82 | 5-(2-Methyl-benzooxazol-6-ylmethylene)-2-(pyridin-3-ylimino)-thiazolidin-4-one | 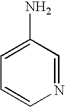 | 337.2 | 1.40 |
| 83 | 3-[5-(2-Methyl-benzooxazol-6-ylmethylene)-4-oxo-thiazolidin-2-ylideneamino]-benzamide | 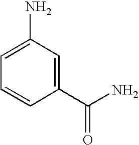 | 379.2 | 1.61 |
| 84 | 2-(2-Hydroxy-ethylimino)-5-(2-methyl-benzooxazol-6-ylmethylene)-thiazolidin-4-one | 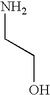 | 304.0 | 1.33 |
| 85 | 2-(1-Hydroxymethyl-2-phenyl-ethylimino)-5-(2-methyl-benzooxazol-6-ylmethylene)-thiazolidin-4-one | 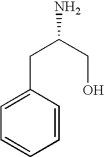 | 394.2 | 1.76 |

### EXAMPLE 86

### N-{6-[2-(2-Bromo-phenylimino)-4-oxo-thiazolidin-5-ylidenemethyl]-1H-benzoimidazol-2-yl}-2-dimethylamino-acetamide

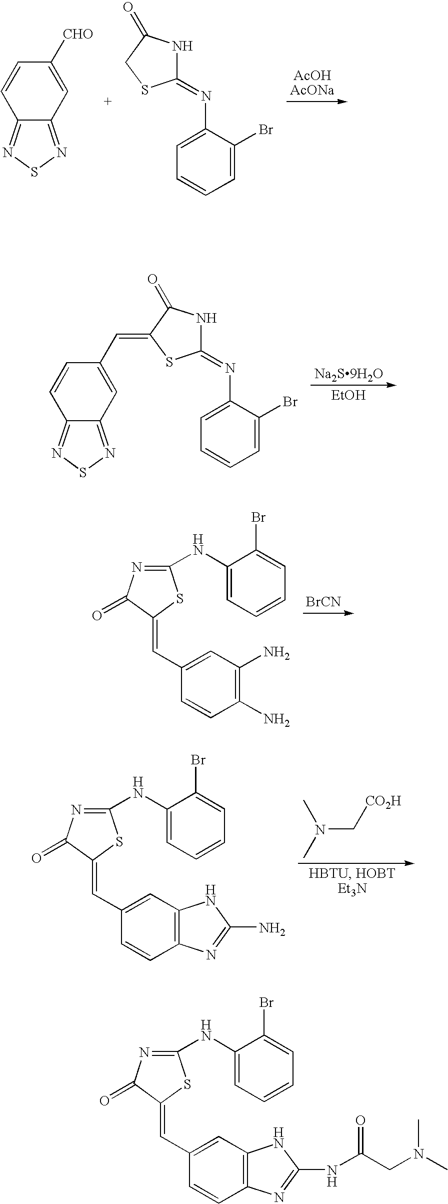

### a. 5-Benzo[1,2,5]thiadiazol-5-ylmethylene-2-(2-bromophenylimino)-thiazolidin-4-one

A mixture of benzo[1,2,5]thiadiazole-5-carbaldehyde (70 mg, 0.43 mmol), (2-bromophenylimino)-thiazolidin-4-one (110 mg, 0.40 mmol), AcONa (100 mg) in AcOH (2 mL) was heated to reflux at 120 degree for 48 hours. After cooling, a small portion of water was added until the solid forms. It was filtered and washed with MeOH, followed by desiccation in vacuo to afford a target product (104 mg, 0.25 mmol). ¹H NMR (DMSO-d₆) δ 7.15 (m, 2H), 7.43 (t, 1H), 7.71 (d, 1H), 7.83 (dd, 1H), 7.89 (s, 1H), 8.16 (d, 1H), 8.22 (s, 1H), 12.83 (sbr, 1H): LC/MS: m/z 417 (M), 419 (M+2)

### b. 2-(2-Bromo-phenylamino)-5-(3,4-diamino-benzylidene)-thiazol-4-one

A mixture 5-benzo[1,2,5]thiadiazol-5-ylmethylene-2-(2-bromophenylimino)-thiazolidin-4-one (380 mg) and Na₂S-9H₂O (600 mg) in ethanol was irradiated by a microwave reactor at 120° C. for 5 hours. The mixture was poured onto aq. NH₄Cl and the formed orange precipitate was filtrated. Washing with H₂O and subsequent desiccation gave 290 mg of the title product. ¹H NMR (DMSO-d₆) δ 4.68 (sbr, 2H), 5.30 (s, 2H), 6.44-6.55 (m, 3H), 7.04 (m, 2H), 7.29 (s, 1H), 7.33 (t, 1H), 7.61 (d, 1H): LC/MS: m/z 389 (M), 391 (M+2).

### c. 5-(2-Amino-3H-benzoimidazol-5-ylmethylene)-2-(2-bromo-phenylimino)-thiazolidin-4-one

A mixture of 2-(2-bromo-phenylamino)-5-(3,4-diamino-benzylidene)-thiazol-4-one (130 mg) and BrCN (120 mg) in methanol (1.5 ml) was heated at 60° C. for 6 h. Treatment with aq. NaOH yielded a precipitate, which then is purified by prep LC-MS to afford the title product (30 mg). ¹H NMR (DMSO-d₆) δ 7.07-7.20 (m, 5H), 7.40 (t, 1H), 7.64 (s, 1H), 7.67 (d, 1H): LC/MS: m/z 414 (M), 416 (M+2)

### d. N-{6-[2-(2-Bromo-phenylimino)-4-oxo-thiazolidin-5-ylidenemethyl]-1H-benzoimidazol-2-yl}-2-dimethylamino-acetamide

A mixture of 5-(2-amino-3H-benzoimidazol-5-ylmethylene)-2-(2-bromo-phenylimino)-thiazolidin-4-one (40 mg), dimethylaminoacetic acid (13 mg), HBTU (45 mg), and triethylamine (25 mg) in dry DMF (1 ml) was stirred at rt for 6 hours. It was washed with water and the formed yellowish solid was collected by filtration. Prep LC-MS purification afforded the title product (10 mg). ¹H NMR (DMSO-d₆) δ 2.30 (s, 6H), 3.24 (s, 2H), 7.10 (m, 2H), 7.29 (m, 1H), 7.39 (m, 1H), 7.46 (m, 1H), 7.63-7.68 (m, 3H): LC/MS: m/z 500 (M+1)

### EXAMPLE 88

### Methyl (5-{(Z)-[2-[(2-bromophenyl)amino]-4-oxo-1,3-thiazol-5(4H)-ylidene]methyl}-1H-benzimidazol-2-yl)carbamate

A mixture of 2-(2-bromo-phenylamino)-5-(3,4-diamino-benzylidene)-thiazol-4-one (88 mg, 0.23 mmol) and 1,3-bis(methoxycarbonyl)methyl-2-thiopsuedourea (46 mg, 0.23 mmol) in dry methanol (1.5 mL) was heated overnight at 60° C. with a air-cooling condenser. The formed yellowish solid was filtered and then washed with H₂O and MeOH to provide the title product (39 mg). ¹H NMR (DMSO-d₆) 3.75 (s, 3H), 7.12-7.16 (m, 2H), 7.28 (d, 1H), 7.41-7.46 (m, 2H), 7.57 (s, 1H), 7.71 (d, 1H), 7.74 (s, 1H), 12.0 (brs, 2H).

### EXAMPLE 89

### (5Z)-2-[(2-chlorophenyl)amino]-5-{[1-(3,3-dimethylbutyl)-1H-benzimidazol-6-yl]methylidene}-1,3-thiazol-4(5H)-one

3-[(3,3-Dimethylbutyl)amino]-4-nitrobenzonitrile. A mixture of 3-(methyloxy)-4-nitrobenzonitrile (1.0 g; 5.6 mmol.) and (3,3-dimethylbutyl)amine (1.5 g; 14.8 mmol.) was stirred and heated at 100° C. for 18 h. The mixture was cooled and the solid mass slurried with hexanes and filtered to afford the title compound (0.75 g; 54%) as a yellow solid. C₁₃H₁₇N₃O₂ requires: % C, 63.2; % H, 6.9; % N, 17.0; Found: % C, 63.1; % H, 6.7; % N, 16.7. 1H NMR (400 MHz, DMSO-d₆) □ ppm 0.97 (s, 9H) 1.53-1.60 (m, 2H) 3.34-3.41 (m, 2H) 7.02 (dd, J=8.72, 1.64 Hz, 1H) 7.59 (d, J=1.77 Hz, 1H) 8.04 (t, J=5.31 Hz, 1H) 8.19 (d, J=8.59 Hz, 1H).

4-Amino-3-[(3,3-dimethylbutyl)amino]benzonitrile. A solution of the compound from Example 89a) (0.69 g; 2.8 mmol.) in ethyl acetate/methanol (3:1) (50.0 mL) was hydrogenated over 10% palladium-on-carbon (100 mg) at room temperature and atmospheric pressure for 30 min. The mixture was filtered through a pad of celite and the filtrate evaporated to afford the title compound (0.60 g; 99%) as a tan oil. 1H NMR (400 MHz, DMSO-d₆) □ ppm 0.92 (s, 9H) 1.62-1.66 (m, 2H) 3.13-3.21 (m, 2H) 6.88 (d, J=8.54 Hz, 1H) 7.31 (dd, J=8.54, 1.79 Hz, 1H) 7.48 (d, J=1.79 Hz, 1H) 8.0-10.5 (br s, 3H).

1-(3,3-Dimethylbutyl)-1H-benzimidazole-6-carbaldehyde. A solution of the compound from Example 89c) (600 mg; 2.8 mmol.) in formic acid (15.0 mL) was stirred and heated under reflux for 1 h. The solution was then cooled to room temperature for the addition of a 50% aqueous suspension of Raney-nickel (1.0 mL) and water (3.0 mL). The mixture was then stirred and heated at 100° C. for 30 min. The mixture was filtered through a pad of celite and evaporated. The residue was treated with water (10.0 mL) then basified with sat. aqu. Sodium hydrogen carbonate and extracted with ethyl acetate (3×50.0 mL). The organic layers were dried and evaporated and the residue purified by flash-chromatography (silica gel, 5% methanol in chloroform) to afford the title compound (380 mg) contaminated with approximately 20% of the [1-(3,3-dimethylbutyl)-1H-benzimidazol-6-yl]methanol. 1H NMR (400 MHz, CHLOROFORM-d) □ ppm 1.09 (s, 9H) 1.81-1.88 (m, 2H) 4.24-4.30 (m, 2H) 7.84 (dd, J=8.34, 1.52 Hz, 1H) 7.93 (d, J=8.34 Hz, 1H) 7.99 (d, J=0.76 Hz, 1H) 8.12 (s, 1H) 10.14 (s, 1H).

(5Z)-2-[(2-chlorophenyl)amino]-5-{[1-(3,3-dimethylbutyl)-1H-benzimidazol-6-yl]methylidene}-1,3-thiazol-4(5H)-one. A solution of the compound from Example 89c) (138 mg; 0.60 mmol.), 2-[(2-chlorophenyl)amino]-1,3-thiazol-4(5H)-one (130 mg; 0.57 mmol.) and piperidine (70 μL; 0.70 mmol.) in ethanol (1.0 mL) was stirred and heated in a microwave reactor at 150° C. for 20 min. The mixture was cooled and filtered to afford the title compound (49.0 mg, 19%) as a pale-yellow powder. C₂₃H₂₃ClN₄OS requires: % C, 62.9; % H, 5.3; % N, 12.8; found: % C, 62.9; % H, 4.9; % N, 12.5. 1H NMR (400 MHz, DMSO-d₆) □ ppm 0.89 (s, 9H) 1.57-1.66 (m, 2H) 4.19-4.29 (m, 2H) 7.15 (dd, J=7.83, 1.26 Hz, 1H) 7.21 (td, J=7.77, 1.39 Hz, 1H) 7.37 (ddd, J=17.94, 8.21, 1.39 Hz, 2H) 7.54 (dd, J=8.08, 1.26 Hz, 1H) 7.66 (s, 1H) 7.73 (d, J=8.34 Hz, 1H) 7.86 (s, 1H) 8.39 (s, 1H) 12.64 (s, 1H).

### EXAMPLE 90

### (5Z)-2-[(2,6-dichlorophenyl)amino]-5-{[1-(3,3-dimethyl butyl)-1H-benzimidazol-6-yl]methylidene}-1,3-thiazol-4(5H)-one, trifluoroacetate salt

Following the procedure of Example 89d) except substituting 2-[(2,6-dichlorophenyl)amino]-1,3-thiazol-4(5H)-one for 2-[(2-chlorophenyl)amino]-1,3-thiazol-4(5H)-one followed by purification by chromatography (ODS silica, gradient 10-100% acetonitrile/water (0.1% TFA)), the title compound was prepared (69.0 mg, 20%). C₂₃H₂₂Cl₂N₄OS. C₂HF₃O₂ requires: % C, 51.1; % H, 3.9; % N, 9.5; found: % C, 50.9; % H, 3.8; % N, 9.4.1H NMR (400 MHz, DMSO-d₆) □ ppm 0.90 (s, 9H) 1.61-1.69 (m, 2H) 4.28-4.39 (m, 2H) 7.23 (t, J=8.21 Hz, 1H) 7.51-7.59 (m, 3H) 7.77-7.87 (m, 2H) 7.95 (s, 1H) 8.87 (s, 1H) 12.97 (s, 1H).

### EXAMPLE 91

### (5Z)-5-{[1-(2-cyclopropylethyl)-1H-benzimidazol-6-yl]methylidene}-2-[(2,6-difluorophenyl)amino]-1,3-thiazol-4(5H)-one

4-amino-3-[(2-cyclopropylethyl)amino]benzonitrile. A mixture of 3-(methyloxy)-4-nitrobenzonitrile (0.5 g; 2.8 mmol.) and (2-cyclopropylethyl)amine (0.24 g; 2.8 mmol.) in DMSO (2.5 mL) was stirred and heated in a microwave reactor at 125° C. for 90 min. The mixture turned bright orange and was diluted with ethyl acetate (20 mL) and washed with sat. aqu. sodium hydrogen carbonate (20 mL) and brine (20 mL). The organic layer was dried over MgSO₄, filtered and rotary evaporated down to residue. The crude residue was dissolved in methanol (10 mL) and ethyl acetate (10 mL) and treated with 10% palladium on carbon (20 mg) and hydrogenated at 25 psi for 1 h. The mixture was filtered through a pad of celite and the filtrate evaporated. Purification by flash-chromatography (silica gel, 5-50% ethyl acetate in hexanes) afforded the title compound (0.230 g; 41%) as a brown crystalline solid. C₁₂H₁₅N₃ MS (ES+) m/e 202 [M+H]⁺

1-(2-cyclopropylethyl)-1H-benzimidazole-6-carbaldehyde. A solution of the compound from Example 91a) (230 mg; 1.14 mmol.) in formic acid (7.0 mL) was stirred and heated under reflux for 2 h. The solution was then cooled to room temperature for the addition of a 50% aqueous suspension of Raney-nickel (1.0 mL) and water (1.0 mL). The mixture was then stirred and heated at 110° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated to afford the title compound (236 mg;) as an impure oil that was used in the next step without further purification. MS (ES+) m/e 215 [M+H]⁺

(5Z)-5-{[1-(2-cyclopropylethyl)-1H-benzimidazol-6-yl]methylidene}-2-[(2,6-difluorophenyl)amino]-1,3-thiazol-4(5H)-one. A solution of the compound from Example 91b) (236 mg; 1.10 mmol.), 2-[(2,6-difluorophenyl)amino]-1,3-thiazol-4(5H)-one (252 mg; 1.10 mmol.) and piperidine (109 μL; 1.10 mmol.) in ethanol (2.0 mL) was stirred and heated in a microwave reactor at 170° C. for 20 min. The mixture was diluted with ethyl acetate (20 mL) and water (10 mL). The organic layer was separated dried and filtered then purified by flash-chromatography (silica gel, 5-50% ethyl acetate in hexanes) to afford the title compound (50.0 mg, 11%) as a pale-yellow powder. C₂₂H₁₈F₂N₄OS MS (ES+) m/e 425 [M+H]⁺. 1H NMR (400 MHz, DMSO-d₆) □ ppm 12.95 (s, 1H) 8.91 (s, 1H) 8.09 (s, 1H) 8.00 (s, 1H) 7.91 (d, J=8.59 Hz, 1H) 7.54 (d, J=8.84 Hz, 1H) 7.29-7.39 (m, 3H) 4.48 (t, J=7.07 Hz, 2H) 1.75-1.82 (m, 2H) 0.69 (s, 1H) 0.37-0.42 (m, 2H) 0.01 (q, J=4.72 Hz, 2H)

### EXAMPLE 92

### (5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt

2-(2-cyclohexylethyl)-1H-isoindole-1,3(2H)-dione. A solution of cyclohexylethanol (20.0 g; 0.156 mol.), triphenylphosphine (40.9 g; 1.1 equiv.) and phthalimide (22.9 g; 1.1 equiv.) in anhydrous tetrahydrofuran (300 mL) was stirred and cooled to 5° C. for the dropwise addition of diisopropyl azodicarboxylate (34.7 g; 1.1 equiv.) in anhydrous tetrahydrofuran (100 mL). The mixture was then stirred at room temperature for 18 h. The mixture was evaporated and the residue washed with diethyl ether (500 mL) and filtered, then the filtrate evaporated and purified by chromatography (silica gel, hexanes/ethyl acetate (4:1)) to afford the title compound (23.8 g; 6-0%) asa colorless oil. 1H NMR (400 MHz, CHLOROFORM-d) □ ppm 0.91-1.02 (m, J=12.06, 11.84, 11.84, 3.16 Hz, 2H) 1.19-1.31 (m, 3H) 1.54-1.63 (m, 2H) 1.64-1.71 (m, 2H) 1.71-1.76 (m, 1H) 1.79 (s, 1H) 1.83 (d, J=2.02 Hz, 1H) 3.69-3.74 (m, 2H) 7.72 (dd, J=5.56, 3.03 Hz, 2H) 7.85 (dd, J=5.43, 2.91 Hz, 2H).

(2-Cyclohexylethyl)amine, hydrochloride salt. A solution of the compound from Example 92a) (23.8 g; 0.092 mol.) and hydrazine hydrate (5.0 mL; 1.1 equiv.) in methanol (250 mL) was stirred and heated under reflux for 3 h. The mixture was cooled and evaporated and the residue slurried with diethyl ether (500 mL) and filtered. The filtrate was then evaporated and dissolved in diethyl ether (200 mL) then the solution saturated with gaseous hydrochloric acid. The mixture was filtered to afford the title compound (1.7 g; 14%) as a yellow powder. 1H NMR (400 MHz, DMSO-d₆) □ ppm 0.83-1.67 (m, 13H), 2.72-2.80 (m, 2H), 8.02 (br s, 3H). 3-[(2-cyclohexylethyl)amino]-4-nitrobenzonitrile. A mixture of the compound from Example 92b) (1.23 g; 7.5 mmol.), 3-(methyloxy)-4-nitrobenzonitrile (1.12 g; 6.3 mmol.) and potassium carbonate (1.1 g; 8.0 mmol.) were well mixed then heated neat at 150° C. with stirring for 18 h. To the resulting cooled, solid mass was added 1M aqu. Hydrochloric acid (50.0 mL) and ethyl acetate (50.0 mL) and the mixture separated and the organic layer dried and evaporated. The residue was purified by chromatography (silica gel, hexanes/ethyl acetate (9:1)) to afford the title compound (0.27 g; 16%).

(5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt. Following the procedures of Examples 91b), 91c) and 91d) except substituting the compound from Example 92c) for the compound from Example 89a) and substituting 2-[(2,6-dichlorophenyl)amino]-1,3-thiazol-4(5H)-one for 2-[(2,6-difluorophenyl)amino]-1,3-thiazol-4(5H)-one followed by purification by chromatography (ODS silica, gradient 10-100% acetonitrile/water (0.1% TFA)), the title compound was prepared (21.1 mg). C₂₅H₂₄Cl₂N₄OS. C₂HF₃O₂ requires: % C, 52.9; % H, 4.1; % N, 9.1; found: % C, 53.4; % H, 4.3; % N, 9.1. 1H NMR (400 MHz, DMSO-d₆) □ ppm 0.85-0.95 (m, 2H) 1.10-1.21 (m, 4H) 1.60-1.71 (m, 7H) 4.26-4.36 (m, 2H) 7.23 (t, J=8.08 Hz, 1H) 7.42 (d, J=8.59 Hz, 1H) 7.56 (d, J=8.08 Hz, 2H) 7.80 (d, J=8.34 Hz, 1H) 7.92-7.96 (m, 2H) 8.77 (s, 1H) 12.93 (s, 1H).

### EXAMPLE 93

### (2Z,5Z)-2-[(2,6-dichlorophenyl)imino]-5-[(2-phenyl-1H-benzimidazol-6-yl)methylidene]-1,3-thiazolidin-4-one, piperidine salt

methyl 2-phenyl-1H-benzimidazole-6-carboxylate. A solution of benzaldehyde (2 mL, 20.0 mmol) and 40% aq. sodium hydrogen sulfite (21 mL) was stirred at room temperature for 1 h. To this mixture is added a solution of methyl 3,4-diaminobenzoate (3.32 g, 20.0 mmol) in ethanol (2 mL). The resulting solution is heated to reflux overnight. The mixture was diluted in water and the resulting precipitate was collected by filtration to obtain 4.90 g of the desired product in 97% yield. The crude was used without further purification. 1H NMR (400 MHz, CHLOROFORM-d) □ ppm 8.35 (s, 1H) 8.06-8.15 (m, 2H) 7.97 (dd, J=8.6, 1.5 Hz, 1H) 7.64 (s, 1H) 7.41-7.50 (m, 3H).

2-phenyl-1H-benzimidazole-5-carbaldehyde. A solution of methyl 2-phenyl-1H-benzimidazole-5-carboxylate (0.300 g, 1.18 mmol) was treated with lithium aluminum hydride (2.4 mL, 2.38 mmol, 1 M solution in THF) under a nitrogen atmosphere at room temperature. The solution was stirred for 1 h and the dumped into ice, treated with saturated aq. ammonium chloride and diluted with brine. Extraction with three volumes of ethyl acetate afforded the title compound as a yellow solid in 54% yield [MS (ES+) m/e 225 [M+H]+. The crude was dissolved in acetone (15 mL) and immediately treated using manganese oxide (1.17 g, 13.4 mmol). The black solution was stirred at room temperature for 36 h. The residual black solid was filtered using a celite pad and washing with three volumes of acetone. The filtrate was concentrated under high vacuum to give a glue-like residue. The residue was washed with three volumes of ether to afford 0.139 g of the desired aldehyde as a yellow powder (52%). The crude material was used without further purification. [MS (ES+) m/e 223 [M+H]+. 1H NMR (400 MHz, MeOD-d₄) □ 10.04 (s, 1H) 8.17 (s, 1H) 8.09-8.16 (m, 2H) 7.86 (dd, J=8.3, 1.5 Hz, 1H) 7.74 (d, J=8.3 Hz, 1H) 7.54-7.61 (m, 3H).

(2Z,5Z)-2-[(2,6-dichlorophenyl)imino]-5-[(2-phenyl-1H-benzimidazol-6-yl)methylidene]-1,3-thiazolidin-4-one. A microwave vial was charged with the compound from example 93b) (0.156 g, 0.702 mmol) and (2Z)-2-[(2,6-dichlorophenyl)imino]-1,3-thiazolidin-4-one (0.183 g, 0.702 mmol) in ethanol. The solution was treated with piperidine (0.07 mL, 0.702 mmol) and the contents were irradiated at 150° C. for 1 h in a microwave reactor. The mixture was allowed to cool to room temperature, taken up in water (15 mL) and extracted with ethyl acetate (3×10 mL). The organic layers were combined, dried over MgSO₄ and evaporated. The crude was purified by flash-chromatography (silica gel, 10% methanol in chloroform) to afford the title compound in 28% yield. [MS (ES+) m/e 465 [M+H]+. 1H NMR (400 MHz, DMSO-d₆) □ ppm 13.09 (s, 1H) 8.17 (d, J=7.3 Hz, 2H) 7.61 (s, 2H) 7.47-7.59 (m, 5H) 7.37 (m, 1H) 7.15 (s, 1H) 3.02 (m, 2H) 1.60-1.68 (m, 2H) 1.51-1.59 (m, 1H).

### EXAMPLE 94

### (5Z)-2-[(2-chlorophenyl)amino]-5-{[1-(2-cyclopropylethyl)-1H-benzimidazol-6-yl]methylidene}-1,3-thiazol-4(5H)-one

Following the procedure of Example 91c) except substituting 2-[(2-chlorophenyl)amino]-1,3-thiazol-4(5H)-one for 2-[(2,6-difluorophenyl)amino]-1,3-thiazol-4(5H)-one followed by purification by flash-chromatography (silica gel, 5-100% ethyl acetate in hex), the title compound was prepared (81.0 mg, 16%). C₂₂H₁₉ClN₄OS MS (ES+) m/e 423 [M+H]⁺. 1H NMR (400 MHz, DMSO-d₆) d ppm 12.70 (bs, 1H) 8.45 (s, 1H) 7.91-7.96 (m, 2H) 7.82 (d, J=8.59 Hz, 1H) 7.64 (dd, J=7.96, 1.14 Hz, 1H) 7.43-7.50 (m, 1H) 7.40 (d, J=8.34 Hz, 1H) 7.28-7.34 (m, 1H) 7.25 (d, J=7.83 Hz, 1H) 4.39 (t, J=6.95 Hz, 2H) 1.68-1.78 (m, 2H) 0.65 (ddd, J=12.19, 7.39, 4.93 Hz, 1H) 0.36-0.44 (m, 2H)-0.01 (q, J=4.80 Hz, 2H).

### EXAMPLE 95

### (5Z)-5-{[1-(2-cyclopropylethyl)-1H-benzimidazol-6-yl]methylidene}-2-[(2,6-dichlorophenyl)amino]-1,3-thiazol-4(5H)-one

Following the procedure of Example 91c) except substituting 2-[(2,6-dichlorophenyl)amino]-1,3-thiazol-4(5H)-one for 2-[(2,6-difluorophenyl)amino]-1,3-thiazol-4(5H)-one followed by purification by chromatography (ODS silica, gradient 10-100% acetonitrile/water (0.1% TFA)), the title compound was prepared (74.0 mg, 18%). C₂₂H₁₈Cl₂N₄OS MS (ES+) m/e 456 [M+H]⁺. 1H NMR (400 MHz, DMSO-d₆) 1H NMR (400 MHz, DMSO-d₆) d ppm 12.98 (s, 1H) 8.79 (s, 1H) 8.04 (s, 1H) 7.99 (s, 1H) 7.88 (d, J=8.59 Hz, 1H) 7.65 (d, J=8.08 Hz, 2H) 7.47 (d, J=8.08 Hz, 1H) 7.32 (t, J=8.08 Hz, 1H) 4.45 (t, J=6.95 Hz, 2H) 1.73-1.81 (m, 2H) 0.67 (s, 1H) 0.35-0.42 (m, 2H)-0.00 (t, J=4.80 Hz, 2H)

### EXAMPLE 96

### (2Z,5Z)-2-[(2,6-dichlorophenyl)imino]-5-{[2-(1-methylethyl)-1H-benzimidazol-6-yl]methylidene}-1,3-thiazolidin-4-one

methyl 2-(1-methylethyl)-1H-benzimidazole-6-carboxylate. A solution of isobutyraldehyde (0.22 mL, 2.41 mmol) and 40% aq. sodium hydrogen sulfite (2.6 mL) was stirred at room temperature for 1 h. To this mixture is added a solution of methyl 3,4-diaminobenzoate (0.400 g, 2.41 mmol) in ethanol (2 mL). The resulting solution is heated to reflux overnight. The mixture was diluted in water and the resulting precipitate was collected by filtration to obtain 0.524 g of the desired product in >99% yield. The crude was used without further purification. [MS (ES+) m/e 219 [M+H]+. 1H NMR (400 MHz, CHLOROFORM-d) [symbol] ppm 8.30 (d, J=1.0 Hz, 1H) 7.93 (dd, J=8.6, 1.5 Hz, 1H) 7.54 (d, J=8.6 Hz, 1H) 3.90 (s, 3H) 3.30-3.41 (m, 1H) 1.48 (d, J=7.1 Hz, 6H).

[2-(1-methylethyl)-1H-benzimidazol-6-yl]methanol. A THF solution of the compound in example 96a) (0.524 g, 2.40 mmol) was treated with lithium aluminum hydride (4.8 mL, 4.80 mmol, 1 M solution in THF) under a nitrogen atmosphere at room temperature. The solution was stirred for 1 h and the dumped into ice, treated with saturated aq. ammonium chloride and diluted with brine. Extraction with three volumes of ethyl acetate afforded 0.354 g of the title compound (78%). [MS (ES+) m/e 191 [M+H]+. 1H NMR (400 MHz, METHANOL-d₄) □ ppm 7.53 (br. s., 1H) 7.44-7.51 (m, 1H) 7.20-7.24 (m, 1H) 4.69 (s, 2H) 3.16-3.27 (m, 1H) 1.44 (d, J=7.1 Hz, 6H).

2-(1-methylethyl)-1H-benzimidazole-6-carbaldehyde. A solution of the compound from example 96b) (0.354 g, 1.86 mmol) in acetone (5 mL) was treated with manganese oxide (1.60 g, 18.6 mmol). The solution was stirred overnight at room temperature. The mixture was filtered using a celite pad and was washed with acetone three times. The combined washings were combined to give 0.212 g of a white solid as the desired compound (61%). [MS (ES+) m/e 189 [M+H]+. 1H NMR (400 MHz, CHLOROFORM-d) □ ppm 10.06 (s, 1H) 8.11 (d, J=1.0 Hz 1H) 7.81 (dd, J=8.3 Hz, 1.5 Hz, 1H) 7.67 (d, J=8.4 Hz, 1H) 3.30-3.41 (m, 1H) 1.53 (d, J=7.0 Hz, 6H).

(2Z,5Z)-2-[(2,6-dichlorophenyl)imino]-5-{[2-(1-methylethyl)-1H-benzimidazol-6-yl]methylidene}-1,3-thiazolidin-4-one. Following the procedure of Example 93c) except substituting 2-(1-methylethyl)-1H-benzimidazole-6-carbaldehyde (0.212 g, 1.13 mmol) for 2-phenyl-1H-benzimidazole-5-carbaldehyde followed by flash-chromatography (silica gel, 10% methanol in chloroform), the title compound was obtained as a yellow solid in 21% yield (0.104 g). [MS (ES+) m/e 431 [M+H]+. 1H NMR (400 MHz, DMSO-d₆) □ ppm 7.84 (br. s., 1H) 7.52-7.66 (m, 4H) 7.30 (dd, J=8.5 Hz, 1.3 Hz1 H) 7.23 (t, J=8.1 Hz, 1H) 3.06-3.21 (m, 1H) 1.32 (d, J=6.8 Hz, 6H).

### EXAMPLE 97

### (2Z,5Z)-2-[(2,6-dichlorophenyl)imino]-5-{[2-(2-methylpropyl)-1H-benzimidazol-6-yl]methylidene}-1,3-thiazolidin-4-one, piperidine salt

methyl 2-(2-methylpropyl)-1H-benzimidazole-5-carboxylate. Following the procedure of Example 96a) except substituting isovaleraldehyde (0.258 g, 3.00 mmol) for isobutyraldehyde and employing aq work up with extraction using ethyl acetate, the title compound was prepared in >99% yield 0.759 g. [MS (ES+) m/e 233 [M+H]+.

[2-(2-methylpropyl)-1H-benzimidazol-5-yl]methanol. Following the procedure employed in Example 96b) except substituting methyl 2-(2-methylpropyl)-1H-benzimidazole-5-carboxylate (0.759 g, 3.27 mmol) for methyl 2-(1-methylethyl)-1H-benzimidazole-6-carboxylate afforded the desired compound as a white solid (0.44 g) in 66% yield. [MS (ES+) m/e 205 [M+H]+.

2-(2-methylpropyl)-1H-benzimidazole-5-carbaldehyde. Following the procedure employed for Example 96c) but substituting [2-(2-methylpropyl)-1H-benzimidazol-5-yl]methanol (0.44 g, 2.15 mmol) for [2-(1-methylethyl)-1H-benzimidazol-6-yl]methanol afforded 0.395 g of the desired compound as a dark orange material in 92% yield. [MS (ES+) m/e 203 [M+H]+.

(2Z,5Z)-2-[(2,6-dichlorophenyl)imino]-5-{[2-(2-methylpropyl)-1H-benzimidazol-6-yl]methylidene}-1,3-thiazolidin-4-one, piperidine salt. Following the procedure for Example 93c) except substituting 2-(2-methylpropyl)-1H-benzimidazole-5-carbaldehyde (0.395 g, 1.95 mmol) for 2-phenyl-1H-benzimidazole-5-carbaldehyde, the desired compound was obtained in 13% (0.11 g) as a yellow powder. [MS (ES+) m/e 445 [M+H]+. 1H NMR (400 MHz, METHANOL-d₄) □ ppm 7.65 (s, 2H) 7.50-7.56 (m, 1H) 7.43 (d, J=8.1 Hz, 2H) 7.32-7.38 (m, 1H) 7.12 (t, J=8.1 Hz, 1H) 3.09-3.14 (m, 4H) 2.75 (d, J=7.3 Hz, 2H) 2.24-2.16 (m, 1H) 1.74-1.82 (m, 4H) 1.65-1.74 (m, 2H) 0.99 (d, J=6.8 Hz, 6H).

### EXAMPLE 98

### (2Z,5Z)-2-[(2,6-dichlorophenyl)imino]-5-{[2-(3-pyridinyl)-1H-benzimidazol-6-yl]methylidene}-1,3-thiazolidin-4 one

methyl 2-(3-pyridinyl)-1H-benzimidazole-5-carboxylate. The procedure for Example 96a) except substituting 3-pyridinecarbaldehyde (0.400 g, 3.70 mmol) for isobutyraldehyde was used. The filtrated residue was purified using flash-chromatography (silica gel, 10% methanol in chloroform) to yield 0.727 g of a yellow solid (78%). [MS (ES+) m/e 254 [M+H]+.

[2-(3-pyridinyl)-1H-benzimidazol-5-yl]methanol. A solution of the compound from Example 98a) (0.727 g, 2.87 mmol.) in THF was treated as the compound in Example 96b). The desired product was obtained as a yellow solid in 87% yield (0.560 g) and was used in the next step without further purification. [MS (ES+) m/e 226 [M+H]+.

2-(3-pyridinyl)-1H-benzimidazole-5-carbaldehyde. A procedure similar for Example 96c) except substituting [2-(3-pyridinyl)-1H-benzimidazol-5-yl]methanol (0.560 g, 2.49 mmol) was used to obtain the desired compound in 20% yield (0.104 g) as a yellow powder. [MS (ES+) m/e 224 [M+H]+.

(2Z,5Z)-2-[(2,6-dichlorophenyl)imino]-5-{[2-(3-pyridinyl)-1H-benzimidazol-6-yl]methylidene}-1,3-thiazolidin-4-one. The procedure for Example 93c) was used except for substituting 2-(3-pyridinyl)-1H-benzimidazole-5-carbaldehyde (0.104 g, 0.466 mmol) instead of 2-phenyl-1H-benzimidazole-5-carbaldehyde. The title compound was obtained as a yellow solid in 28% yield (0.061 g) after work up and purification. [MS (ES+) m/e 466 [M+H]+. 1H NMR (400 MHz, DMSO-d₆) □ ppm 13.43 (d, J=46.5 Hz, 1H) 12.89 (br. s., 1H) 9.34 (br. s., 1H) 8.65-8.75 (m, 1H) 8.50 (s, 1H) 7.75-7.96 (m, 2H) 7.68 (s, 1H) 7.54-7.64 (m, 3H) 7.42 (m, 1H) 7.25 (t, J=8.2 Hz, 1H).

### EXAMPLE 99

### (2Z,5Z)-2-[(2,6-dichlorophenyl)imino]-5-{[2-(hydroxymethyl)-1H-benzimidazol-5-yl]methylidene}-1,3-thiazolidin-4-one

methyl 2-({[(1,1-dimethylethyl)(dimethyl)silyl]oxy}methyl)-1H-benzimidazole-5-carboxylate. The procedure used for Example 96a) was used except substituting {[(1,1-dimethylethyl)(dimethyl)silyl]oxy}acetaldehyde (1.00 g, 5.70 mmol) for isobutyraldehyde. After diluting with water the mixture was extracted using three volumes of ethyl acetate. The combined organic portions were dried over magnesium sulfate and the whole was filtrated and concentrated. The crude was purified using flash chromatography (silica gel, 60% Ethyl acetate, hexane) to obtain a mixture of the desired TBS protected alcohol 99a (0.02 g) [MS (ES+) m/e 321 [M+H]+ and the deprotected alcohol 99a2) (0.386 g) for 22% yield [MS (ES+) m/e 207 [M+H]+.

[2-({[(1,1-dimethylethyl)(dimethyl)silyl]oxy}methyl)-1H-benzimidazol-5-yl]methanol. A solution of the compound from Example 99a) (0.02 g, 0.062 mmol.) in dichloromethane (3.00 mL) was kept at −78° C. and treated with diisobutylaluminum hydride (0.075 mL, 0.075 mmol) dropwise. After 1 h, the solution was quenched with methanol while keeping the temperature at −78° C. The cold bath is removed and the mixture was treated with sat. aq. Rochelle salt. The solution was stirred for 1 h, then extracted using three volumes of ethyl acetate, dried over magnesium sulfate, filtrated and concentrated to afford 58% yield. The crude was used without further purification in the next step. [MS (ES+) m/e 293 [M+H]+].

2-({[(1,1-dimethylethyl)(dimethyl)silyl]oxy}methyl)-1H-benzimidazole-5-carbaldehyde. Following the procedure for Example 96c) but substituting 99b) (0.02 g, 0.07 mmol) for the compound in 96c), the desired compound was obtained as an oil in >99% yield [MS (ES+) m/e 291 [M+H]+.

(2Z,5Z)-2-[(2,6-dichlorophenyl)imino]-5-{[2-({[(1,1-dimethylethyl)(dimethyl)silyl]oxy}methyl)-1H-benzimidazol-5-yl]methylidene}-1,3-thiazolidin-4-one. A microwave vial was charged with 2-({[(1,1-dimethylethyl)(dimethyl)silyl]oxy}methyl)-1H-benzimidazole-5-carbaldehyde (0.0312 g, 0.11 mmol), (2Z)-2-[(2,6-dichlorophenyl)imino]-1,3-thiazolidin-4-one (0.029 g, 0.11 mmol), piperidine (0.02 mL, 0.11 mol) and ethanol (1 mL). The contents were irradiated at 150° C. for 1 h. The resulting solution was treated with 1 N HCl (5 ml) and the resulting precipitate was filtered, washed with 5×5 mL portions of water and dried under high vacuum to afford 0.032 g (55%) of the desired material. The crude was used without further purification. [MS (ES+) m/e 534 [M+H]+.

(2Z,5Z)-2-[(2,6-dichlorophenyl)imino]-5-{[2-(hydroxymethyl)-1H-benzimidazol-5-yl]methylidene}-1,3-thiazolidin-4-one. A solution of the compound in Example 99d) (0.032 g, 0.06 mmol) was taken up in a 1:1 mixture of THF:water (2 mL) and was treated with aq. acetic acid (0.004 mL, 0.06 mL). The resulting solution was stirred overnight at room temperature. The mixture was diluted in 3 mL of ethyl acetate and extracted three times. The combined organic portions were washed with sat. aq. sodium bicarbonate, dried over magnesium sulfate, filtrated and concentrated to afford the title compound as a yellow solid in 38% yield (9.6 mg). [MS (ES+) m/e 419 [M+H]+. 1H NMR (400 MHz, METHANOL-d₄) □ ppm 7.85 (s, 1H) 7.69 (s, 1H) 7.61 (d, J=8.3 Hz, 1H) 7.44-7.51 (m, 2H) 7.39 (dd, J=8.5, 1.1 Hz, 1H) 7.18 (t, J=8.2 Hz, 1H) 4.84 (s, 2H).

### EXAMPLE 100

### (2Z,5Z)-2-[(2,6-dichlorophenyl)imino]-5-{[2-(2-hydroxyethyl)-1H-benzimidazol-5-yl]methylidene}-1,3-thiazolidin-4-one

methyl 2-(2-{[(1,1-dimethylethyl)(dimethyl)silyl]oxy}ethyl)-1H-benzimidazole-5-carboxylate. Using the procedure for Example 96a) except substituting 3-{[(1,1-dimethylethyl)(dimethyl)silyl]oxy}propanal (2.00 g, 10.6 mmol) for isobutyraldehyde, followed by purification using flash chromatography (silica gel, 60% ethyl acetate/hexane) the desired product 100a1) was obtained in 42% yield, along with the deprotected alcohol 100a2) (0.205 g, 8%). The protected alcohol 100a1) was carried on to the next step [MS (ES+) m/e 335 [M+H]+].

[2-(2-{[(1,1-dimethylethyl)(dimethyl)silyl]oxy}ethyl)-1H-benzimidazol-5-yl]methanol. A solution of the compound in Example 100a1) (0.210 g, 0.0.63 mmol) was taken up in dichloromethane (3 mL) and treated with diisobutylaluminum hydride (0.63 mL, 0.63 mmol) at −78° C. The mixture was allowed to reach room temperature over 1 h. Then it was quenched with methanol (1 mL), treated with sat. aq. Rochelle salt (10 mL) and the slurry was stirred overnight. Extraction with 3×10 mL ethyl acetate, drying over magnesium sulfate and concentration under high vacuum afforded 0.25 g (>99%) of the title compound. The crude was used without further purification [MS (ES+) m/e 307 [M+H]+].

2-({2-[(1,1-dimethylethyl)(dimethyl)silyl]oxy}ethyl)-1H-benzimidazole-5-carbaldehyde. Following the same procedure as in Example 96c) but substituting Example 100b) (0.25 g, 0.81 mmol) for the compound in Example 96c), The desired product was obtained 38% yield. The crude was used in the next step. [MS (ES+) m/e 305 [M+H]+]

(2Z,5Z)-2-[(2,6-dichlorophenyl)imino]-5-{[2-(2-hydroxyethyl)-1H-benzimidazol-5-yl]methylidene}-1,3-thiazolidin-4-one. A microwave vial was charged with the compound from Example 100c) (0.095 g, 0.31 mmol), (2Z)-2-[(2,6-dichlorophenyl)imino]-1,3-thiazolidin-4-one (0.082 g, 0.31 mmol), piperidine (0.03 mL, 0.31 mmol) and ethanol (3 mL). The vial was sealed and irradiated for 1 h at 150° C. in a microwave reactor. The crude was treated with aq. 1 N hydrochloric acid (3 mL) and the resulting precipitate was filtered off. The remaining solid was washed with water and dried under high vacuum. The desired compound was obtained after flash chromatography (silica gel, 10% methanol, dichloromethane) as a brown solid in 26% yield (0.035 g) [MS (ES+) m/e 433 [M+H]+. 1H NMR (400 MHz, DMSO-d₆) □ ppm 13.03 (br. s., 1H) 7.94 (s, 1H) 7.88 (s, 1H) 7.84 (d, J=8.6 Hz, 1H) 7.62 (d, J=7.6 Hz, 1H) 7.58 (d, J=8.1 Hz, 2H) 7.25 (t, J=8.2 Hz, 1H) 3.88 (t, J=5.7 Hz, 2H) 3.22 (t, J=5.8 Hz, 2H).

### EXAMPLE 101

### (2Z,5Z)-2-[(2,6-dichlorophenyl)imino]-5-{[2-(2-pyridinyl)-1H-benzimidazol-6-yl]methylidene}-1,3-thiazolidin-4-one

methyl 2-(2-pyridinyl)-1H-benzimidazole-5-carboxylate. A solution of 2-pyridinecarbaldehyde (0.19 g, 1.80 mmol) in 40% aq. sodium hydrogen sulfite was stirred for 1 h at room temperature before being added to a solution of methyl 3,4-diaminobenzoate (0.300 g, 1.80 mmol) in ethanol (3 mL). The resulting mixture was stirred under reflux overnight. Then it was taken in 10 mL of water and extracted with 3×10 mL of ethyl acetate, dried over magnesium sulfate, filtrated and concentrated. Flash chromatography (silica gel, 10% methanol, dichloromethane) afforded the desired compound as white powder in very low yield (13%) [MS (ES+) m/e 254 [M+H]+].

[2-(2-pyridinyl)-1H-benzimidazol-5-yl]methanol. Following the procedure from Example 96a) but substituting 101a) (0.058 g, 0.23 mmol) for 96a), the desired compound was obtained in quantitative yield. The crude was used in the following step [MS (ES+) m/e 226 [M+H]+].

2-(2-pyridinyl)-1H-benzimidazole-5-carbaldehyde. Following the same procedure used in Example 96c) but substituting 96b) with 101b) (0.052 g, 0.23 mmol), the desired product was obtained as a yellow solid in 40% yield. The crude was used without further purification [MS (ES+) m/e 224 [M+H]+].

(2Z,5Z)-2-[(2,6-dichlorophenyl)imino]-5-{[2-(2-pyridinyl)-1H-benzimidazol-6-yl]methylidene}-1,3-thiazolidin-4-one. A microwave vial was charged with the compound from Example 100c) (0.02 g, 0.09 mmol), (2Z)-2-[(2,6-dichlorophenyl)imino]-1,3-thiazolidin-4-one (0.023 g, 0.09 mmol), piperidine (0.01 mL, 0.09 mL) and ethanol (2 mL). The vial was irradiated for 2 h at 150° C. in a microwave reactor. The solution was cooled to room temperature and diluted in water (4 mL). The aq. phase was extracted using 3×4 mL ethyl acetate. The combined organic portions were dried over magnesium sulfate, filtrated and concentrated to afford the desired product as a yellow solid in 81% yield. [MS (ES+) m/e 466 [M+H]+]. 1H NMR (400 MHz, DMSO-d₆) □ ppm 13.33 (d, J=71.7 Hz, 1H) 12.88 (br. s., 1H) 8.75 (s, 1H) 8.31-8.36 (m, 1H) 8.02 (t, J=7.7 Hz, 1H) 7.84-7.90 (m, 1H) 7.79 (d, J=8.3 Hz, 1H) 7.69 (s, 1H) 7.52-7.65 (m, 3H) 7.42 (d, J=8.8 Hz, 1H) 7.25 (t, J=8.1 Hz, 1H).

### EXAMPLE 102

### (5Z)-5-{[1-(2-cyclopentylethyl)-1H-benzimidazol-6-yl]methylidene}-2-[(2,6-dichlorophenyl)amino]-1,3-thiazol-4(5H)-one

4-amino-3-[(2-cyclopentylethyl)amino]benzonitrile. A mixture of 3-(methyloxy)-4-nitrobenzonitrile (1.77 g; 9.9 mmol.) and (2-cyclopentylethyl)amine (4 mL; excess.) in DMSO (2.5 mL) was stirred and heated in a microwave reactor at 125° C. for 65 min. The mixture turned bright orange and was diluted with ethyl acetate (50 mL) and washed with sat. aqu. sodium hydrogen carbonate (50 mL) and brine (50 mL). The organic layer was dried over MgSO₄, filtered and rotary evaporated down to residue. Following purification by flash-chromatography (silica gel, 5-100% ethyl acetate in hexanes), the crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.400 g; 18%) as a brown solid. MS (ES+) m/e 230 [M+H]⁺

1-(2-cyclopentylethyl)-1H-benzimidazole-6-carbaldehyde. A solution of the compound from Example 102a) (400 mg; 1.76 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated with the major product being [1-(2-cyclopentylethyl)-1H-benzimidazol-6-yl]methanol. The crude alcohol was dissolved in acetone (5 mL), treated with manganese dioxide (300 mg) and stirred at room temperature for 3 h. The mixture was filtered through a pad of celite and evaporated to afford the title compound (300 mg;) as an oil that was used in the next step without further purification. MS (ES+) m/e 243 [M+H]⁺

(5Z)-5-{[1-(2-cyclopentylethyl)-1H-benzimidazol-6-yl]methylidene}-2-[(2,6-dichlorophenyl)amino]-1,3-thiazol-4(5H)-one. A solution of the compound from Example 102b) (120 mg; 0.496 mmol.), 2-[(2,6-dichlorophenyl)amino]-1,3-thiazol-4(5H)-one (129 mg; 0.496 mmol.) and piperidine (49 μL; 0.496 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (21.0 mg, 8%) as a pale-yellow powder. C₂₄H₂₂Cl₂N₄OS MS (ES+) m/e 485 [M+H]⁺. 1H NMR (400 MHz, DMSO-d₆) d ppm 12.91 (s, 1H) 8.72 (s, 1H) 7.92 (s, 2H) 7.80 (d, J=8.34 Hz, 1H) 7.56 (d, J=8.08 Hz, 2H) 7.40 (d, J=8.84 Hz, 1H) 7.23 (t, J=8.21 Hz, 1H) 4.29 (t, J=7.20 Hz, 2H) 1.76-1.84 (m, 2H) 1.67 (m, 2H) 1.51-1.60 (m, 3H) 1.40-1.49 (m, 2H) 1.07 (m, 2H)

### EXAMPLE 103

### (5Z)-2-[(2-chlorophenyl)amino]-5-{[1-(2-cyclopentylethyl)-1H-benzimidazol-6-yl]methylidene}-1,3-thiazol-4(5H)-one

Following the procedure of Example 102c) except substituting 2-[(2-chlorophenyl)amino]-1,3-thiazol-4(5H)-one for 2-[(2,6-dichlorophenyl)amino]-1,3-thiazol-4(5H)-one, the title compound was prepared (15.0 mg, 8%). C₂₄H₂₃ClN₄OS MS (ES+) m/e 451 [M+H]⁺. 1H NMR (400 MHz, DMSO-d₆) d ppm 12.88 (s, 1H) 8.68 (s, 1H) 7.92 (s, 1H) 7.88 (s, 1H) 7.79 (d, J=8.08 Hz, 1H) 7.55 (d, J=7.33 Hz, 1H) 7.35-7.44 (m, 2H) 7.22 (s, 2H) 4.28 (s, 2H) 1.79 (s, 2H) 1.67 (s, 3H) 1.55 (s, 2H) 1.44 (s, 2H) 1.07 (s, 2H)

### EXAMPLE 104

### (2Z,5Z)-2-[(2,6-dichlorophenyl)imino]-5-[(2-methyl-1H-benzimidazol-6-yl)methylidene]-1,3-thiazolidin-4-one

methyl 2-methyl-1H-benzimidazole-6-carboxylate. Following the procedure as in compound 101a) except substituting with acetaldehyde (0.10 mL, 1.81 mmol) instead of 2-pyridinecarbaldehyde, followed by flash chromatography (silica gel, 60% ethyl acetate, hexane) the desired compound was obtained as a yellow residue in 45% yield. [MS (ES+) m/e 191 [M+H]+]. (2-methyl-1H-benzimidazol-6-yl)methanol. To a solution of 104a) (0.156 g, 0.820 mmol) in THF (5 mL) is held at 0° C. while adding lithium aluminum hydride (1.00 mL, 0.984 mmol). The solution was allowed to reach room temperature over 2 h. Then the mixture was dumped in water followed by treatment with Rochelle salt (5 mL). After extraction with ethyl acetate (3×10 mL) the washings were combined, dried and filtrated to yield 0.102 g (77%) of the desired compound. The crude was taken up to the next step. [MS (ES+) m/e 163 [M+H]+].

2-methyl-1H-benzimidazole-6-carbaldehyde. Following the procedure in Example 96c) but using 104b) (0.102 g, 0.63 mmol) instead of 96b), the desired compound was delivered in 60% yield. The crude was used without further purification. [MS (ES+) m/e 161 [M+H]+].

(2Z,5Z)-2-[(2,6-dichlorophenyl)imino]-5-[(2-methyl-1H-benzimidazol-6-yl)methylidene]-1,3-thiazolidin-4-one. Following the procedure for Example 100d) but substituting 100c) with 2-methyl-1H-benzimidazole-6-carbaldehyde (0.069 g, 0.38 mmol) the desired compound was prepared in 73% yield as a yellow solid. [MS (ES+) m/e 403 [M+H]+]. 1H NMR (400 MHz, METHANOL-d₄) □ ppm 7.87 (s, 1H) 7.79 (s, 1H) 7.75 (d, J=8.6 Hz, 1H) 7.62 (d, J=8.6 Hz, 1H) 7.47 (d, J=8.1 Hz, 2H) 7.19 (t, J=8.2 Hz, 1H) 2.81 (s, 3H).

### EXAMPLE 105

### (2Z,5Z)-2-[(2,6-dichlorophenyl)imino]-5-{[2-(4-pyridinyl)-1H-benzimidazol-5-yl]methylidene}-1,3-thiazolidin-4-one

methyl 2-(4-pyridinyl)-1H-benzimidazole-5-carboxylate. Following the procedure for 101a) except substituting 2-pyridinecarbaldehyde with 4-pyridinecarbaldehyde (0.19 g, 1.77 mmol) the desired compound was obtained in 60% yield. The crude was used in the next step. [MS (ES+) m/e 254 [M+H]+].

[2-(4-pyridinyl)-1H-benzimidazol-5-yl]methanol. The same procedure used in Example 104b) except substituting 104a) for 105a) (0.43 g, 1.70 mmol) afforded the desired compound in 46% yield. The crude was used without further purification. [MS (ES+) m/e 226 [M+H]+].

2-(4-pyridinyl)-1H-benzimidazole-5-carbaldehyde. The procedure used for Example 96c) but using 105b) (0.175 g, 0.78 mmol) instead of 96b) was employed to prepare the desired compound. After concentration the compound was obtained in 74% yield. The crude was carried on to the next step. [MS (ES+) m/e 224 [M+H]+]. (2Z,5Z)-2-[(2,6-dichlorophenyl)imino]-5-{[2-(4-pyridinyl)-1H-benzimidazol-5-yl]methylidene}-1,3-thiazolidin-4-one. The procedure used to prepare Example 100) was used to prepare the desired compound. Substituting of 100c) for 105c) (0.065 g, 0.29 mmol) afforded 31% of a deep orange solid. [MS (ES+) m/e 466 [M+H]+]. 1H NMR (400 MHz, DMSO-d₆) □ ppm 12.94 (br. s., 1H) 8.88 (d, J=6.3 Hz, 2H) 8.28 (d, J=6.1 Hz, 2H) 7.93 (s, 1H) 7.79-7.85 (m, 2H) 7.60 (d, J=8.3 Hz, 2H) 7.45-7.51 (m, 1H) 7.26 (t, J=8.2 Hz, 1H)

### EXAMPLE 106

### (2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one

3-[(2-cyclopropylethyl)amino]-4-nitrobenzonitrile. A microwave vial was charged with 3-(methyloxy)-4-nitrobenzonitrile (0.430 g, 2.4 mmol) and 2-cyclopropylethanamine (2.00 g, 24 mmol), sealed and irradiated at 125° C. for 4000 seconds. The solution was diluted in ethyl acetate (10 mL) and washed with sat. aq. sodium hydrogen carbonate. Flash chromatography yielded the desired compound as a bright orange solid in 56% yield. [MS (ES+) m/e 232 [M+H]+].

4-amino-3-[(2-cyclopropylethyl)amino]benzonitrile. A solution of 106a) (0.313 g, 1.35 mmol) in ethyl acetate (100 mL) was hydrogenated over 20% w/w palladium-on-carbon ((0.003 g, 0.27 mmol) at room temperature and 40 psi of hydrogen (g). The mixture was filtered through a celite pad and evaporated to afford the title compound as an orange oil (95%). The crude was used without further purification. [MS (ES+) m/e 202 [M+H]+].

1-(2-cyclopropylethyl)-2-(3-pyridinyl)-1H-benzimidazole-6-carbonitrile. The procedure used in Example 96a) except substituting isobutyraldehyde for 3-pyridinecarbaldehyde (0.139 mL, 1.48 mmol) and methyl 3,4-diaminobenzoate for 106b) (0.298 g, 1.48 mmol). The resulting precipitate was filtered off and washed with water. The phases were separated and the organic phase was dried over magnesium sulfate, filtrated and concentrated to give the desired product in 92% yield as a yellow solid. [MS (ES+) m/e 289 [M+H]+].

1-(2-cyclopropylethyl)-2-(3-pyridinyl)-1H-benzimidazole-6-carbaldehyde. A solution of 115c) (0.406 g, 1.41 mmol) in formic acid (10 mL) was treated with an aq. suspension of Raney-Ni (1 mL, 2800 slurry in water). The resulting mixture was refluxed for 1 h. The mixture was filtered, washed with ethanol, diluted in water (15 mL), alkalinized using portions of sodium carbonate, extracted with 3×15 mL dichloromethane, dried and concentrated. The desired aldehyde was obtained after flash chromatography (silica, 60% ethyl acetate, hexane) in 10% yield. [MS (ES+) m/e 292 [M+H]+].

(2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one. Following the procedure for Example 100d) except substituting 100c) for 106d) (0.040 g, 0.137 mmol) the desired compound was obtained as a yellow solid (0.028 g, 38%). 1H NMR (400 MHz, DMSO-d₆) □ ppm 12.94 (br. s., 1H) 8.79 (d, J=4.3 Hz, 1H) 8.34 (d, J=8.1 Hz, 1H) 8.06 (td, J=7.8, 1.8 Hz, 1H) 7.97 (br. s., 1H) 7.94 (s, 1H) 7.85 (d, J=8.3 Hz, 1H) 7.55-7.62 (m, 3H) 7.50 (d, J=8.1 Hz, 1H) 7.44 (d, J=8.3 Hz, 1H) 7.24 (t, J=8.2 Hz, 1H) 4.87 (t, J=7.1 Hz, 2H) 1.67 (q, J=7.1 Hz, 2H) 0.47-0.65 (m, 1H) 0.16-0.31 (m, 2H)-0.11 (q, J=4.9 Hz, 2H).

### EXAMPLE 107

### (2Z,5Z)-2-[(2,6-dichlorophenyl)imino]-5-{[1-methyl-2-(3-pyridinyl)-1H-benzimidazol-5-yl]methylidene}-1,3-thiazolidin-4-one

1-methyl-2-(3-pyridinyl)-1H-benzimidazole-5-carbonitrile. The procedure used in Example 96a) except substituting methyl 3,4-diaminobenzoate for 4-amino-3-(methylamino)benzonitrile (0.102 g, 0.69 mmol) and isobutyraldehyde for 3-pyridinecarbaldehyde (0.06 mL, 0.69 mmol) was used to prepare the desired compound as a white solid in 80% yield. The crude was used without further purification. [MS (ES+) m/e 235 [M+H]+].

1-methyl-2-(3-pyridinyl)-1H-benzimidazole-5-carbaldehyde. A similar procedure used to prepare 115d) was employed except substituting 115c) with 117c) (0.130 g, 0.55 mmol). The crude was purified using flash chromatography (silica gel, 60% ethyl acetate, hexane) to yield a colorless oil as the desired product in 17% yield. The crude was immediately treated with manganese oxide (0.079 g, 0.91 mmol) at room temperature for 12 h. The mixture was filtered off through a celite pad. The filtrate was concentrated to afford the desired product as a colorless solid in quantitative yield. [MS (ES+) m/e 238 [M+H]+].

(2Z,5Z)-2-[(2,6-dichlorophenyl)imino]-5-{[1-methyl-2-(3-pyridinyl)-1H-benzimidazol-6-yl]methylidene}-1,3-thiazolidin-4-one. The procedure used in Example 100d) except substituting 100c) for 107e) (0.023 g, 0.097 mmol) was used. After treatment with aq. 1 N hydrochloric acid and extraction with 3×5 mL ethyl acetate, drying over magnesium sulfate, filtration and concentration, the desired product was obtained as a deep orange solid in 27% yield. [MS (ES+) m/e 480 [M+H]+]. 1H NMR (400 MHz, METHANOL-d₄) □ ppm 9.02 (br. s., 1H) 8.78 (br. s., 1H) 8.26-8.38 (m, 1H) 7.66-7.81 (m, 3H) 7.57 (d, J=8.3 Hz, 2H) 7.37-7.52 (m, 4H) 7.17 (t, J=8.1 Hz, 1H) 3.38 (d, J=7.3 Hz, 3H).

### EXAMPLE 108

### (2Z,5Z)-5-{[2-(aminomethyl)-1H-benzimidazol-5-yl]methylidene}-2-[(2,6-dichlorophenyl)imino]-1,3-thiazolidin-4-one

methyl 2-(azidomethyl)-1H-benzimidazole-5-carboxylate. A solution of the deprotected alcohol 99a2) (0.150 g, 0.73 mmol) in THF (10 mL) was treated with {[bis(phenyloxy)phosphanyl]oxy}azide (0.22 mL, 1.02 mmol) at 0° C. After 5 min 1,8-diazabicyclo[5.4.0]undec-7-ene (0.132 g, 0.88 mmol) was added and the mixture was stirred for 2 h at 0° C., then at room temperature for 20 h. The mixture was diluted in 10 mL of ethyl acetate, quenched using 10 mL of water and extracted 3×10 mL ethyl acetate. The organic layer was dried over sodium sulfate, filtrated and concentration. After purification (silica gel, 60% ethyl acetate, hexane) the desired product was obtained in quantitative yield. [MS (ES+) m/e 232 [M+H]+].

methyl 2-(aminomethyl)-1H-benzimidazole-5-carboxylate. To a solution of 108a) (0.170 g, 0.737 mmol) in 12 mL of tetrahydrofuran was added triphenylphosphine (0.270 g, 1.03 mmol). After 2 min water (2 mL) was added and the mixture was stirred at room temperature for 3 h. Then it was treated with aq. 28% ammonium hydroxide (2 ml) and stirred for an additional 1 h. The mixture was separated into layers and the aq. portion was extracted with 3×10 mL of ethyl acetate. The combined organic portions were dried over magnesium sulfate, filtrated, concentrated and purified (silica gel, 60% ethyl acetate, hexane). The desired product was obtained in 38% yield. [MS (ES+) m/e 206 [M+H]+].

1,1-dimethylethyl {[5-(hydroxymethyl)-1H-benzimidazol-2-yl]methyl}carbamate. To a solution of 108b) (0.058 g, 0.28 mmol) in dimethylformamide (2 mL) was added BOC-anhydride (0.300 mL, 0.28 mmol) and triethylamine (0.04 mL. 0.28 mmol). The solution was irradiated at 150° C. for 300 sec in a microwave reactor (90% LCMS yield, [MS (ES+) m/e 306 [M+H]+]). The mixture was evaporated and the crude was immediately dissolved in 2 mL of tetrahydrofuran and treated with lithium aluminum hydride (0.30 mL, 0.28 mmol, 1 M solution in tetrahydrofuran). The mixture was stirred overnight and then quenched with 3 mL of methanol. The salts were washed with a sat. aq. solution of Rochelle salt (5 mL) and stirred for an additional 1 h. The solution was extracted using 3×10 mL ethyl acetate. The combined organic layers were dried over magnesium sulfate, filtrated and concentrated to give the desired product as a yellow oil in quantitative yield. 1H NMR (400 MHz, CHLOROFORM-d) □ ppm 7.29-7.36 (m, 1H) 7.08 (d, J=8.3 Hz, 1H) 6.33 (t, J=5.8 Hz, 1H) 4.65 (s, 2H) 4.41 (d, J=5.8 Hz, 2H) 2.06 (s, 1H) 1.38 (s, 9H).

1,1-dimethylethyl[(5-formyl-1H-benzimidazol-2-yl)methyl]carbamate. A solution of 108c) (0.09 g, 0.32 mmol) in ethyl acetate (5 mL) was treated with manganese oxide (0.28 g, 3.20 mmol) at room temperature for 2 h. The mixture was filtered using a celite pad. After concentration, the desired product was obtained as a colorless oil in 58% yield. [MS (ES+) m/e 276 [M+H]+].

(2Z,5Z)-5-{[2-(aminomethyl)-1H-benzimidazol-5-yl]methylidene}-2-[(2,6-dichlorophenyl)imino]-1,3-thiazolidin-4-one. A microwave vial was charged with 108d) (0.047 g, 0.170 mmol), (2Z)-2-[(2,6-dichlorophenyl)imino]-1,3-thiazolidin-4-one (0.044 g, 0.170 mmol), piperidine (0.17 mL, 0.170 mmol), dissolved in ethanol (3 mL). The mixture was irradiated at 150° C. for 3600 sec, cooled and treated with aq. 1 N hydrochloric acid. The precipitate was collected by filtration, washed with water and dried under vacuum. The orange powder was immediately dissolved in ethyl acetate (5 mL) and treated with aq. 3 N hydrochloric acid for 18 h. The mixture was separated into layers by dissolving in 5 mL of water and extracted with 3×5 mL of ethyl acetate. The combined organic portions were neutralized using sat. aq. sodium hydrogen carbonate, the dried over magnesium sulfate, filtrated and concentrated to give a yellow solid as the final product (41% yield). [MS (ES+) m/e 418 [M+H]+]. 1H NMR (400 MHz, METHANOL-d₄) □ ppm 7.84 (s, 1H) 7.71 (s, 1H) 7.62 (s, 1H) 7.47 (d, J=8.3 Hz, 2H) 7.39 (s, 1H) 7.18 (t, J=8.2 Hz, 1H) 4.65 (br. s., 2H).

### EXAMPLE 109

### (2Z,5Z)-5-(1H-benzimidazol-5-ylmethylidene)-2-[(2,6-dichlorophenyl)imino]-1,3-thiazolidin-4-one

methyl 1H-benzimidazole-5-carboxylate. Thionyl Chloride (0.135 mL, 1.85 mmol) was added dropwise to a solution of 1H-benzimidazole-5-carboxylic acid (0.300 g, 1.85 mmol) in methanol (50.0 mL). The solution was refluxed for 12 h and then cooled to room temperature. The mixture was slurried into sat. aq. sodium hydrogen carbonate, separated into layers and extracted using ethyl acetate (3×15 mL). The combined organic layers are dried over magnesium sulfate, filtrated and concentrated to give a purple solid as the desired product in 90% yield. The crude was used without further purification. [MS (ES+) m/e 177 [M+H]+].

1H-benzimidazol-5-ylmethanol. A solution of 109a) (0.056 g, 0.317 mmol) was treated as 104a) in Example 104b). The desired product was obtained as a pink oil in >99% yield. The crude was used immediately. [MS (ES+) m/e 149 [M+H]+]. 1H-benzimidazole-5-carbaldehyde. A solution of 109b) (0.069 g, 0.466 mmol) in acetone was treated as 107d) in Example 107e). The desired product was obtained as a white powder in 48% yield. [MS (ES+) m/e 147 [M+H]+].

(2Z,5Z)-5-(1H-benzimidazol-5-ylmethylidene)-2-[(2,6-dichlorophenyl)imino]-1,3-thiazolidin-4-one. Following the procedure in Example 100d) substituting 100c) for 109c) (0.033 g, 0.22 mmol). After filtration, washing and drying under vacuum the desired product was obtained as a bright yellow powder in 19% yield. [MS (ES+) m/e 389 [M+H]+]. 1H NMR (400 MHz, DMSO-d₆) □ ppm 12.94 (br. s., 1H) 8.87 (s, 1H) 7.91 (s, 1H) 7.85 (s, 1H) 7.80 (d, J=8.6 Hz, 1H) 7.58 (d, J=8.1 Hz, 2H) 7.52 (dd, J=8.6, 1.0 Hz, 1H) 7.24 (t, J=8.1 Hz, 1H).

### EXAMPLE 110

### (2Z,5Z)-2-[(2,6-dichlorophenyl)imino]-5-{[2-(hydroxymethyl)-1-methyl-1H-benzimidazol-6-yl]methylidene}-1,3-thiazolidin-4-one

3-(methylamino)-4-nitrobenzoic acid. A microwave vial was charged with 3-(methyloxy)-4-nitrobenzoic acid (1.00 g, 5.07 mmol) and potassium carbonate (1.40 g, 10.1 mmol). Then water (2 mL) was added followed by methylamine (2.50 ml, 5.07 mmol, 2 M solution in methanol). The vial was irradiated at 160° C. during 300 seconds. The solution was allowed to reach room temperature and dissolved in ethyl acetate. The resulting precipitate was collected by filtration to give 68% of the desired product as a red-orange solid. The crude was used without further purification. [MS (ES+) m/e 197 [M+H]+].

4-amino-N-methyl-3-(methylamino)-N-(methyloxy)benzamide. A mixture of 110a) (0.552 g, 2.81 mmol) and thionyl chloride (1.65 mL) in excess was heated to 80° C. for 18 h and then concentrated under reduced pressure. Dry toluene was added and the mixture was evaporated (3 times). The acyl chloride was taken up in dichloromethane (20 mL) and the solution was cooled to 0° C. Then pyridine (0.68 mL, 8.43 mmol) and N,O-dimethylhydroxylamine hydrochloride (0.411 g, 4.22 mmol) were added and the solution was allowed to reach room temperature overnight. The resulting solution was diluted in dichloromethane, separated into layers and washed twice with brine. The combined organic washings were dried over sodium sulfate, filtrated and concentrated. The crude was immediately dissolved in ethanol (50 mL), transferred to a hydrogenation vessel and treated with 20% palladium-over-carbon (0.10 g, 0.10 mmol). The solution was purged under a stream of nitrogen and then exposed to 50 psi of hydrogen for 1.5 h. The mixture was filtrated and concentration under reduced pressure afforded the desired compound as a yellow oil in 58% yield. [MS (ES+) m/e 210 [M+H]+].

2-(hydroxymethyl)-N,1-dimethyl-N-(methyloxy)-1H-benzimidazole-6-carboxamide. A solution of sodium metabisulfite (0.10 g, 0.53 mmol) in water (0.5 mL) was introduced directly into a mixture of 110c) (0.22 g, 1.06 mmol) and {[(1,1-dimethylethyl)(dimethyl)silyl]oxy}acetaldehyde (0.20 mL, 1.06 mmol) in ethanol (5 mL). The solution was stirred overnight under reflux and then allowed to cool down to room temperature. The mixture was concentrated and the residue was extracted using ethyl acetate, dried over magnesium sulfate, filtrated and concentrated to give 4% of the desired alcohol as a clear oil. [MS (ES+) m/e 250 [M+H]+].

2-(hydroxymethyl)-1-methyl-1H-benzimidazole-6-carbaldehyde. To a solution of 110d) (0.012 g, 0.046 mmol) in dry tetrahydrofuran (1 mL) at −60° C. was added lithium aluminum hydride (0.05 mL, 0.046 mmol). Stirring was continued at −60° C. for 1 h and then methanol was added until bubbling ceased, followed by sat. aq. solution of Rochelle salt (2 mL). The solution was stirred overnight and then it was separated into layers and extracted using 3×5 mL ethyl acetate. The organic phase was dried over magnesium sulfate, filtrated and concentrated to give a yellow powder in 75% yield. 1H NMR (400 MHz, CHLOROFORM-d) □ ppm 10.14 (s, 1H) 8.07 (s, 1H) 7.95 (s, 2H) 5.16 (s, 2H) 4.06 (s, 3H).

(2Z,5Z)-2-[(2,6-dichlorophenyl)imino]-5-{[2-(hydroxymethyl)-1-methyl-1H-benzimidazol-6-yl]methylidene}-1,3-thiazolidin-4-one. The procedure employed for Example 100d) was use but substituting 100c) for 110e) (0.006 g, 0.032 mmol). The expected product was obtained in 26% yield as a yellow solid. [MS (ES+) m/e 43.3 [symbol] [symbol] [symbol] [symbol] ppm 7.91 (s, 1H) 7.64-7.72 (m, 2H) 7.47 (d, J=8.1 Hz, 2H) 7.40 (d, J=8.6 Hz, 1H) 7.18 (t, J=8.2 Hz, 1H) 4.88 (s, 2H) 3.93 (s, 3H).

### EXAMPLE 111

### (5Z)-2-[(2,6-dichlorophenyl)amino]-5-({1-[2-(3-pyridinyl)ethyl]-1H-benzimidazol-6-yl}methylidene)-1,3-thiazol-4(5H)-one

4-amino-3-{[2-(3-pyridinyl)ethyl]amino}benzonitrile. A mixture of 3-(methyloxy)-4-nitrobenzonitrile (1.0 g; 5.62 mmol.) and [2-(3-pyridinyl)ethyl]amine (754 mg; 6.18 mmol.) in DMSO (1.0 mL) was stirred and heated in a microwave reactor at 125° C. for 65 min. The mixture turned bright brown-orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.394 g; 29%) as a light brown solid which was used in the next step without further purification. MS (ES+) m/e 239 [M+H]⁺

1-[2-(3-pyridinyl)ethyl]-1H-benzimidazole-6-carbaldehyde. A solution of the compound from Example 111a) (394 mg; 1.66 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. sodium hydrogen carbonate and extracted with dichloromethane (2×25.0 mL). The organic layers were dried and evaporated to afford the title compound as a mixture with its corresponding alcohol as a minor product (222 mg) as an oil that was used in the next step without further purification. MS (ES+) m/e 252 [M+H]⁺

(5Z)-2-[(2,6-dichlorophenyl)amino]-5-({1-[2-(3-pyridinyl)ethyl]-1H-benzimidazol-6-yl}methylidene)-1,3-thiazol-4(5H)-one. A solution of the compound from Example 111b) (222 mg; 0.884 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (6.0 mg, 3%) as a pale-yellow powder. C₂₄H₁₇Cl₂N₅OS MS (ES+) m/e 493 [M+H]⁺. 1H NMR (400 MHz, DMSO-d₆) d ppm 12.99 (bs, 1H) 8.64-8.73 (m, 3H) 8.09-8.16 (m, 2H) 7.89 (s, 1H) 7.81 (d, J=8.59 Hz, 1H) 7.70-7.76 (m, 1H) 7.57 (d, J=8.34 Hz, 2H) 7.37 (d, J=8.84 Hz, 1H) 7.23 (t, J=8.08 Hz, 1H) 4.65 (t, J=7.07 Hz, 2H) 3.31 (t, J=7.07 Hz, 2H)

### EXAMPLE 112

### (5Z)-5-{[1-(cyclopropylmethyl)-1H-benzimidazol-6-yl]methylidene}-2-[(2,6-dichlorophenyl)amino]-1,3-thiazol-4(5H)-one

4-amino-3-(cyclopropylmethylamino)benzonitrile. A mixture of 3-(methyloxy)-4-nitrobenzonitrile (1.0 g; 5.62 mmol.) and cyclopropylmethylamine (438 mg; 6.18 mmol.) in DMSO (1.0 mL) was stirred and heated in a microwave reactor at 125° C. for 65 min. The mixture turned bright orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.317 g; 30%) as a light brown solid which was used in the next step without further purification. MS (ES+) m/e 188 [M+H]⁺

1-(cyclopropylmethyl)-1H-benzimidazole-6-carbaldehyde. A solution of the compound from Example 112a) (317 mg; 1.67 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated to afford the title compound as a mixture with its corresponding alcohol as a minor product (182 mg) as an oil that was used in the next step without further purification. MS (ES+) m/e 201 [M+H]⁺

(5Z)-5-{[1-(cyclopropylmethyl)-1H-benzimidazol-6-yl]methylidene}-2-[(2,6-dichlorophenyl)amino]-1,3-thiazol-4(5H)-one. A solution of the compound from Example 112b) (182 mg; 0.910 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (4.0 mg, 3%) as a orange powder. C₂₁H₁₆Cl₂N₄OS MS (ES+) m/e 443 [M+H]⁺.

### EXAMPLE 113

### (5Z)-5-[(1-cyclopentyl-1H-benzimidazol-6-yl)methylidene]-2-[(2,6-dichlorophenyl)amino]-1,3-thiazol-4(5H)-one

4-amino-3-(cyclopentylamino)benzonitrile. A mixture of 3-(methyloxy)-4-nitrobenzonitrile (1.0 g; 5.62 mmol.) and cyclopentylamine (526 mg; 6.18 mmol.) in DMSO (1.0 mL) was stirred and heated in a microwave reactor at 125° C. for 65 min. The mixture turned orange and a precipitate formed upon cooling and was filtered off and dried. The crude residue was dissolved in methanol (10 mL) and ethyl acetate (10 mL) and treated with 10% palladium on carbon (20 mg) and hydrogenated at 40 psi for 1 h. The mixture was filtered through a pad of celite and the filtrate evaporated to give the title compound (0.331 g; 29%) as a light brown solid which was used in the next step without further purification. MS (ES+) m/e 202 [M+H]⁺

1-cyclopentyl-1H-benzimidazole-6-carbaldehyde. A solution of the compound from Example 113a) (331 mg; 1.65 mmol.) in formic acid (10.0 mL) was stirred and heated under reflux for 2 h. The solution was then cooled to room temperature for the addition of a 50% aqueous suspension of Raney-nickel (2.0 mL) and water (2.0 mL). The mixture was then stirred and heated at 70° C. for 45 min. The mixture was cooled to 45° C. and then filtered through a pad of celite and evaporated. The residue was diluted with water (5.0 mL) then taken to pH=8 with sat. aqu. Sodium hydrogen carbonate and extracted with dichloromethane (2×25.0 mL). The organic layers were dried and evaporated to afford the title compound as a mixture with its corresponding alcohol as a minor product (206 mg) as an oil that was used in the next step without further purification. MS (ES+) m/e 215 [M+H]⁺

(5Z)-5-[(1-cyclopentyl-1H-benzimidazol-6-yl)methylidene]-2-[(2,6-dichlorophenyl)amino]-1,3-thiazol-4(5H)-one. A solution of the compound from Example 111b) (206 mg; 0.962 mmol.), 2-[(2,6-dichlorophenyl)amino]-1,3-thiazol-4(5H)-one (100 mg; 0.385 mmol.) and piperidine (38 μL; 0.385 mmol.) in ethanol (2.0 mL) was stirred and heated in a microwave reactor at 150° C. for 20 min. The mixture was by purified directly by chromatography (ODS silica, gradient 10-100% acetonitrile/water (0.1% TFA)) to afford the title compound (5.0 mg, 3%) as a yellow powder. C₂₂H₁₈Cl₂N₄OS MS (ES+) m/e 457 [M+H]⁺.

### EXAMPLE 114

### (5Z)-5-(1,3-Benzoxazol-6-ylmethylidene)-2-[(2,6-dichlorophenyl)amino]-1,3-thiazol-4(5H)-one

(5Z)-2-[(2,6-Dichlorophenyl)amino]-5-[(3-hydroxy-4-nitrophenyl)methylidene]-1,3-thiazol-4(5H)-one. A mixture of 2-[(2,6-dichlorophenyl)amino]-1,3-thiazol-4(5H)-one (2.61 g; 0.01 mol.), 3-hydroxy-4-nitrobenzaldehyde (1.67 g; 0.01 mol.) and piperidine (1.0 mL; 0.01 mol.) in ethanol (5.0 mL) was stirred and heated in a microwave reactor at 150° C. for 20 min. The mixture was cooled and poured into 1M aqu. hydrochloric acid (50.0 mL) then extracted with ethyl acetate (200 mL). The organic layer was dried and evaporated to afford the title compound (3.2 g; 78%) as an orange powder. C₁₆H₉Cl₂N₃O₄S requires: % C, 46.9; % H, 2.2; % N, 10.2; found: % C, 46.9; % H, 2.1; % N, 10.0. 1H NMR (400 MHz, DMSO-d₆) □ ppm 7.14 (dd, J=8.59, 1.52 Hz, 1H) 7.20 (d, J=1.52 Hz, 1H) 7.24 (t, J=8.21 Hz, 1H) 7.58 (d, J=8.08 Hz, 2H) 7.69 (s, 1H) 7.94 (d, J=8.59 Hz, 1H) 11.31 (s, 1H) 13.11 (s, 1H).

(5Z)-5-[(4-Amino-3-hydroxyphenyl)methylidene]-2-[(2,6-dichlorophenyl)amino]-1,3-thiazol-4(5H)-one. A solution of the compound from Example 113a) (3.1 g; 7.5 mmol.) in methanol (200 mL) was hydrogenated over 10% w/w palladium-on-carbon (0.60 g) at room temperature and atmospheric pressure for 20 h. The mixture was filtered through celite and evaporated to afford the title compound (2.85 g; quantitative). 1H NMR (400 MHz, DMSO-d₆) □ ppm 5.46 (s, 2H) 6.61 (d, J=8.34 Hz, 1H) 6.74 (s, 1H) 6.83 (d, J=8.34 Hz, 1H) 7.19 (s, 1H) 7.35-7.45 (m, 1H) 7.53 (d, J=7.33 Hz, 2H) 9.49 (s, 1H) 12.55 (s, 1H).

(5Z)-5-(1,3-Benzoxazol-6-ylmethylidene)-2-[(2,6-dichlorophenyl)amino]-1,3-thiazol-4(5H)-one. A solution of the compound from Example 113b) (0.42 g; 1.1 mmol.) in triethyl orthoformate (2.0 mL) was stirred and heated in a microwave reactor at 125° C. for 15 min. The mixture was cooled and directly purified by chromatography (silica gel, hexanes/ethyl acetate (7:3)) to afford the title compound (161 mg; 38%) as a cream powder. C₁₇H₉Cl₂N₃O₂S requires: % C, 52.3; % H, 2.3; % N, 10.8; found: % C, 52.0; % H, 2.3; % N, 10.1. 1H NMR (400 MHz, DMSO-d₆) □ ppm 7.24 (t, J=8.08 Hz, 1H) 7.51 (dd, J=8.34, 1.26 Hz, 1H) 7.58 (d, J=8.08 Hz, 2H) 7.86-7.94 (m, 2H) 8.00 (d, J=1.26 Hz, 1H) 8.88 (s, 1H) 13.00 (s, 1H).

### EXAMPLE 115 CAPSULE COMPOSITION

An oral dosage form for administering the present invention is produced by filing a standard two piece hard gelatin capsule with the ingredients in the proportions shown in Table I, below.

TABLE I

| INGREDIENTS | AMOUNTS |
|---|---|
| (5Z)-2-[(2-Chlorophenyl)amino]-5-[(1-methyl-1H- | 25 mg |
| benzimidazol-6-yl)methylidene]-1,3-thiazol-4(5H)-one | |
| (compound of Ex. 1) | |
| Lactose | 55 mg |
| Talc | 16 mg |
| Magnesium Stearate | 4 mg |

### EXAMPLE 116

### Injectable Parenteral Composition

An injectable form for administering the present invention is produced by stirring 1.5% by weight of (5Z)-2-[(2-Chlorophenyl)amino]-5-[(1,2-dimethyl-1H-benzimidazol-6-yl)methylidene]-1,3-thiazol-4(5H)-one (compound of Ex. 9) in 10% by volume propylene glycol in water.

### EXAMPLE 117

### Tablet Composition

The sucrose, calcium sulfate dihydrate and an Akt inhibitor as shown in Table II below, are mixed and granulated in the proportions shown with a 10% gelatin solution. The wet granules are screened, dried, mixed with the starch, talc and stearic acid, screened and compressed into a tablet.

TABLE II

| INGREDIENTS | AMOUNTS |
|---|---|
| (5Z)-2-[(2,6-dichlorophenyl)amino]-5-({1-[2-(3- | 20 mg |
| pyridinyl)ethyl]-1H-benzimidazol-6-yl}methylidene)-1,3- | |
| thiazol-4(5H)-one (compound of Ex. 111) | |
| calcium sulfate dihydrate | 30 mg |
| sucrose | 4 mg |
| starch | 2 mg |
| talc | 1 mg |
| stearic acid | 0.5 mg |

While the suitable embodiments of the invention are illustrated by the above, it is to be understood that the invention is not limited to the precise instructions herein disclosed and that the right to all modifications coming within the scope of the following claims is reserved.

What is claimed is:

1. A method of inhibiting one or more phosphatoinositides 3-kinases (PI3Ks) in a mammal; comprising administering to the mammal a therapeutically effective amount of a compound of Formula (I): 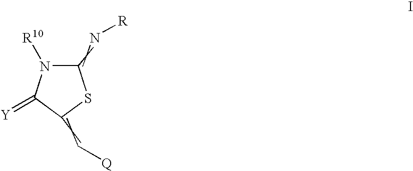
wherein:
R is selected from: hydrogen, aryl, substituted aryl, cycloalkyl, substituted cycloalkyl, C₁₋₆alkyl and substituted C₁₋₆alkyl;
R¹⁰ is selected from: hydrogen, C₁₋₆alkyl, —(CH₂)ₘOH and —(CH₂)ₘCOOH,
where m is 0 to 6;
Y is selected from: ═O, ═S and ═NR¹¹,
where R¹¹ is selected from: hydrogen, C₁₋₆alkyl, —(CH₂)ₚOH and —(CH₂)ₚCOOH,
where p is 0 to 6; and
Q is a radical or substituted radical of the formula, 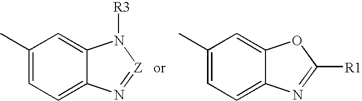
in which Z is N or C—R²;
wherein R² is hydrogen, —NH₂, —C₁₋₆alkyl, substituted —C₁₋₆alkyl, —CF₃, aryl or a radical or substituted radical of the formula 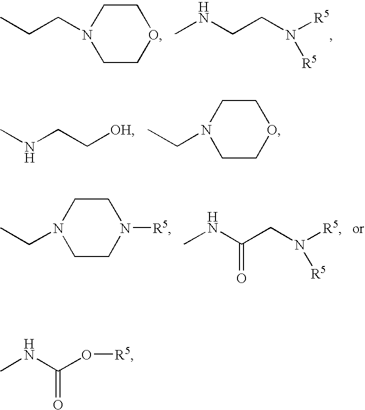
wherein R⁵ is selected from: hydrogen, —C₁₋₆alkyl and substituted —C₁₋₆alkyl; and
R³ is hydrogen, —C₁₋₆alkyl, substituted —C₁₋₆alkyl or C₃₋₁₂cycloalkyl; and
R¹ is hydrogen, —C₁₋₆alkyl, substituted —C₁₋₆alkyl, amino, mono substituted amino, disubstituted amino and trifluoromethyl,
and/or a pharmaceutically acceptable salt, hydrate, solvate or pro-drug thereof.

2. A method of treating one or more disease state selected from the group consisting of: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, in a mammal, which method comprises administering to such mammal, a therapeutically effective amount of a compound according to claim 1.

3. A method of treating cancer comprises co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

4. The method of claim 3, wherein the disease state is selected from the group consisting of: multiple sclerosis, psoriasis, rheumatoid arthritis, systemic lupus erythematosis, inflammatory bowel disease, lung inflammation, thrombosis, brain infection/inflammation, meningitis and encephalitis.

5. The method of claim 3, wherein the disease state is selected from the group consisting of: Alzheimer's disease, Huntington's disease, CNS trauma, stroke and ischemic conditions.

6. The method of claim 3, wherein the disease state is selected from the group consisting of: atherosclerosis, heart hypertrophy, cardiac myocyte dysfunction, elevated blood pressure and vasoconstriction.

7. The method of claim 3, wherein the disease state is selected from the group consisting of: chronic obstructive pulmonary disease, anaphylactic shock fibrosis, psoriasis, allergic diseases, asthma, stroke, ischemia-reperfusion, platelets aggregation/activation, skeletal muscle atrophy/hypertrophy, leukocyte recruitment in cancer tissue, antiogenesis, invasion metastasis, melanoma, Karposi's sarcoma, acute and chronic bacterial and viral infections, sepsis, transplantation rejection, graft rejection, glomerulo sclerosis, glomerulo nephritis, progressive renal fibrosis, endothelial and epithelial injuries in the lung, and lung airways inflammation.

8. The method of claim 3 wherein the disease is cancer.

9. The method of claim 3 wherein the disease is selected from a group consisting of: ovarian cancer, pancreatic cancer, breast cancer, prostate cancer and leukemia.

10. The method of claim 3 wherein the mammal is human.

11. The method of claim 1, wherein said PI3 kinase is a PI3α.

12. The method of claim 1, wherein said PI3 kinase is a PI3γ.

13. The method of claim 1, wherein said compound is selected from:
(5Z)-2-[(2-Chlorophenyl)amino]-5-[(1-methyl-1H-benzimidazol-6-yl)methylidene]-1,3-thiazol-4(5H)-one;
(5Z)-2-[(2,6-dichlorophenyl)amino]-5-[(1-methyl-1H-benzimidazol-6-yl)methylidene]-1,3-thiazol-4(5H)-one;
(5Z)-2-[(2,6-difluorophenyl)amino]-5-[(1-methyl-1H-benzimidazol-6-yl)methylidene]-1,3-thiazol-4(5H)-one;
(5Z)-2-[(2,4-dimethylphenyl)amino]-5-[(1-methyl-1H-benzimidazol-6-yl)methylidene]-1,3-thiazol-4(5H)-one;
(5Z)-5-[(1-methyl-1H-benzimidazol-6-yl)methylidene]-2-{[2-(methyloxy)phenyl]amino}-1,3-thiazol-4(5H)-one;
(5Z)-5-[(1-methyl-1H-benzimidazol-6-yl)methylidene]-2-{[2-(trifluoromethyl)phenyl]amino}-1,3-thiazol-4(5H)-one;
(5Z)-2-[(2,4-difluorophenyl)amino]-5-[(1-methyl-1H-benzimidazol-6-yl)methylidene]-1,3-thiazol-4(5H)-one;
(5Z)-2-[(2-chloro-4-fluorophenyl)amino]-5-[(1-methyl-1H-benzimidazol-6-yl)methylidene]-1,3-thiazol-4(5H)-one;
(5Z)-2-[(2-Chlorophenyl)-amino]-5-[(1,2-dimethyl-1H-benzimidazol-6-yl)-methylidene]-1,3-thiazol-4(5H)-one;
(5Z)-2-[(2,6-dichlorophenyl)amino]-5-[(1,2-dimethyl-1H-benzimidazol-6-yl)methylidene]-1,3-thiazol-4(5H)-one;
(5Z)-2-[(2,6-difluorophenyl)amino]-5-[(1,2-dimethyl-1H-benzimidazol-6-yl)methylidene]-1,3-thiazol-4(5H)-one;
(5Z)-5-[(1,2-dimethyl-1H-benzimidazol-6-yl)methylidene]-2-[(2,4-dimethylphenyl)amino]-1,3-thiazol-4(5H)-one;
(5Z)-2-[(2,4-difluorophenyl)amino]-5-[(1,2-dimethyl-1H-benzimidazol-6-yl)methylidene]-1,3-thiazol-4(5H)-one;
(5Z)-2-[(2-chloro-4-fluorophenyl)amino]-5-[(1,2-dimethyl-1H-benzimidazol-6-yl)methylidene]-1,3-thiazol-4(5H)-one;
(5Z)-2-[(2-Chlorophenyl)-amino]-5-({1-[2-(4-morpholinyl)ethyl]-1H-benzimidazol-6-yl}-methylidene)-1,3-thiazol-4(5H)-one;
(5Z)-2-[(2,6-dichlorophenyl)amino]-5-({1-[2-(4-morpholinyl)ethyl]-1H-benzimidazol-6-yl}methylidene)-1,3-thiazol-4(5H)-one;
(5Z)-2-[(2-chloro-4-fluorophenyl)amino]-5-({1-[2-(4-morpholinyl)ethyl]-1H-benzimidazol-6-yl}methylidene)-1,3-thiazol-4(5H)-one;
(5Z)-2-[(2-chlorophenyl)amino]-5-({1-[2-(dimethylamino)ethyl]-1H-benzimidazol-6-yl}methylidene)-1,3-thiazol-4(5H)-one;
(5Z)-2-[(2,6-dichlorophenyl)amino]-5-({1-[2-(dimethylamino)ethyl]-1H-benzimidazol-6-yl}methylidene)-1,3-thiazol-4(5H)-one;
(5Z)-2-[(2,4-difluorophenyl)amino]-5-({1-[2-(dimethylamino)ethyl]-1H-benzimidazol-6-yl}methylidene)-1,3-thiazol-4(5H)-one;
(5Z)-5-({1-[2-(dimethylamino)ethyl]-1H-benzimidazol-6-yl}methylidene)-2-(phenylamino)-1,3-thiazol-4(5H)-one;
(5Z)-2-[(2-chlorophenyl)amino]-5-({1-[2-(diethylamino)ethyl]-1H-benzimidazol-6-yl}methylidene)-1,3-thiazol-4(5H)-one;
(5Z)-2-[(2,6-dichlorophenyl)amino]-5-({1-[2-(diethylamino)ethyl]-1H-benzimidazol-6-yl}methylidene)-1,3-thiazol-4(5H)-one;
(5Z)-2-[(2-chlorophenyl)amino]-5-({1-[3-(4-morpholinyl)propyl]-1H-benzimidazol-6-yl}methylidene)-1,3-thiazol-4(5H)-one;
(5Z)-2-[(2,6-dichlorophenyl)amino]-5-({1-[3-(4-morpholinyl)propyl]-1H-benzimidazol-6-yl}methylidene)-1,3-thiazol-4(5H)-one;
(5Z)-2-[(2-chlorophenyl)amino]-5-({1-[3-(4-methyl-1-piperazinyl)propyl]-1H-benzimidazol-6-yl}methylidene)-1,3-thiazol-4(5H)-one;
(5Z)-2-[(2,6-dichlorophenyl)amino]-5-({1-[3-(4-methyl-1-piperazinyl)propyl]-1H-benzimidazol-6-yl}methylidene)-1,3-thiazol-4(5H)-one;
(5Z)-2-[(2-chlorophenyl)amino]-5-({1-[2-(1-pyrrolidinyl)ethyl]-1H-benzimidazol-6-yl}methylidene)-1,3-thiazol-4(5H)-one;
(5Z)-2-[(2,6-dichlorophenyl)amino]-5-({1-[2-(1-pyrrolidinyl)ethyl]-1H-benzimidazol-6-yl}methylidene)-1,3-thiazol-4(5H)-one;
(5Z)-2-[(2-chlorophenyl)amino]-5-({1-[2-(1-piperidinyl)ethyl]-1H-benzimidazol-6-yl}methylidene)-1,3-thiazol-4(5H)-one;
(5Z)-2-[(2,6-dichlorophenyl)amino]-5-({1-[2-(1-piperidinyl)ethyl]-1H-benzimidazol-6-yl}methylidene)-1,3-thiazol-4(5H)-one;
(5Z)-2-[(2,6-difluorophenyl)amino]-5-({1-[2-(1-piperidinyl)ethyl]-1H-benzimidazol-6-yl}methylidene)-1,3-thiazol-4(5H)-one;
(5Z)-2-[(2-Chlorophenyl)amino]-5-({1-[2-(dimethylamino)ethyl]-2-methyl-1H-benzimidazol-6-yl}methylidene)-1,3-thiazol-4(5H)-one;
(5Z)-2-[(2,6-dichlorophenyl)amino]-5-({1-[2-(dimethylamino)ethyl]-2-methyl-1H-benzimidazol-6-yl}methylidene)-1,3-thiazol-4(5H)-one;
(5Z)-2-[(2,4-difluorophenyl)amino]-5-({1-[2-(dimethylamino)ethyl]-2-methyl-1H-benzimidazol-6-yl}methylidene)-1,3-thiazol-4(5H)-one;
(5Z)-5-({1-[2-(dimethylamino)ethyl]-2-methyl-1H-benzimidazol-6-yl}methylidene)-2-(phenylamino)-1,3-thiazol-4(5H)-one;
(5Z)-2-[(2,6-dichlorophenyl)amino]-5-{[1-(2-hydroxyethyl)-2-methyl-1H-benzimidazol-6-yl]methylidene}-1,3-thiazol-4(5H)-one;
(5Z)-2-[(2-chlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-dichlorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2,6-difluorophenyl)amino]-5-({2-methyl-1-[2-(1-piperidinyl)ethyl]-1H-benzimidazol-6-yl}methylidene)-1,3-thiazol-4(5H)-one;
(5Z)-2-[(2-chlorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2,6-dichlorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2,4-difluorophenyl)amino]-5-[(1-methyl-2-{[2-(4-morpholinyl)ethyl]amino}-1H-benzimidazol-6-yl)methylidene]-1,3-thiazol-4(5H)-one;
(5Z)-2-[(2-chlorophenyl)amino]-5-[(2-{[2-(dimethylamino)ethyl]amino}-1-methyl-1H-benzimidazol-6-yl)methylidene]-1,3-thiazol-4(5H)-one;
(5Z)-2-[(2-chlorophenyl)amino]-5-({2-[(2-hydroxyethyl)amino]-1-methyl-1H-benzimidazol-6-yl}methylidene)-1,3-thiazol-4(5H)-one;
(5Z)-2-[(2-chlorophenyl)amino]-5-{[1-methyl-2-(4-morpholinyl methyl)-1H-benzimidazol-6-yl]methylidene}-1,3-thiazol-4(5H)-one;
(5Z)-2-[(2,6-dichlorophenyl)amino]-5-{[1-methyl-2-(4-morpholinyl methyl)-1H-benzimidazol-6-yl]methylidene}-1,3-thiazol-4(5H)-one;
(5Z)-2-[(2-chlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2,6-dichlorophenyl)amino]-5-({1-methyl-2-[(4-methyl-1-piperazinyl)methyl]-1H-benzimidazol-6-yl}methylidene)-1,3-thiazol-4(5H)-one;
(5Z)-2-[(2-Chlorophenyl)-amino]-5-{[1-methyl-2-(trifluoromethyl)-1H-benzimidazol-6-yl]-methylidene}-1,3-thiazol-4(5H)-one;
(5Z)-2-[(2,6-dichlorophenyl)amino]-5-{[1-methyl-2-(trifluoromethyl)-1H-benzimidazol-6-yl]methylidene}-1,3-thiazol-4(5H)-one;
(5Z)-2-[(2,6-dichlorophenyl)amino]-5-{[1-[2-(dimethylamino)ethyl]-2-(trifluoromethyl)-1H-benzimidazol-6-yl]methylidene}-1,3-thiazol-4(5H)-one;
(5Z)-2-[(2-chlorophenyl)amino]-5-{[2-(1,1-dimethylethyl)-1-methyl-1H-benzimidazol-6-yl]methylidene}-1,3-thiazol-4(5H)-one;
(5Z)-2-[(2,6-dichlorophenyl)amino]-5-{[2-(1,1-dimethylethyl)-1-methyl-1H-benzimidazol-6-yl]methylidene}-1,3-thiazol-4(5H)-one;
(5Z)-2-[(2-Chlorophenyl)amino]-5-[(1-methyl-1H-1,2,3-benzotriazol-6-yl)methylidene]-1,3-thiazol-4(5H)-one;
(5Z)-2-[(2,6-dichlorophenyl)amino]-5-[(1-methyl-1H-1,2,3-benzotriazol-6-yl)methylidene]-1,3-thiazol-4(5H)-one;
(5Z)-2-[(2-chlorophenyl)amino]-5-({1-[2-(dimethylamino)ethyl]-1H-1,2,3-benzotriazol-6-yl}methylidene)-1,3-thiazol-4(5H)-one;
(5Z)-2-[(2,6-dichlorophenyl)amino]-5-({1-[2-(dimethylamino)ethyl]-1H-1,2,3-benzotriazol-6-yl}methylidene)-1,3-thiazol-4(5H)-one;
2-(2,6-Dichloro-phenylimino)-5-(2-methyl-benzooxazol-6-yl-methylene)-thiazolidin-4-one;
2-(2,6-Difluoro-phenylimino)-5-(2-methyl-benzooxazol-6-yl-methylene)-thiazolidin-4-one;
2-(2-Fluoro-phenylimino)-5-(2-methyl-benzooxazol-6-yl-methylene)-thiazolidin-4-one;
2-(2-Chloro-phenylimino)-5-(2-methyl-benzooxazol-6-ylmethylene)-thiazolidin-4-one;
2-(2-Trifluromethyl-phenylimino)-5-(2-methyl-benzooxazol-6-ylmethylene)-thiazolidin-4-one;
2-(2,4-Difluoro-phenylimino)-5-(2-methyl-benzooxazol-6-ylmethylene)-thiazolidin-4-one;
2-(2,5-Dichloro-phenylimino)-5-(2-methyl-benzooxazol-6-ylmethylene)-thiazolidin-4-one;
2-(2,4-Dimethyl-phenylimino)-5-(2-methyl-benzooxazol-6-ylmethylene)-thiazolidin-4-one;
2-(4-Cyano-phenylimino)-5-(2-methyl-benzooxazol-6-ylmethylene)-thiazolidin-4-one;
4-[5-(2-Methyl-benzooxazol-6-ylmethylene)-4-oxo-thiazolidin-2-ylideneamino]-benzoic acid;
2-(2,4-Dichloro-phenylimino)-5-(2-methyl-benzooxazol-6-ylmethylene)-thiazolidin-4-one;
2-(2,5-Difluoro-phenylimino)-5-(2-methyl-benzooxazol-6-ylmethylene)-thiazolidin-4-one;
5-(2-Methyl-benzooxazol-6-ylmethylene)-2-phenylimino-thiazolidin-4-one;
5-(2-Methyl-benzooxazol-6-ylmethylene)-2-(2-piperidin-1-yl-ethylimino)-thiazolidin-4-one;
2-(2-Methoxy-ethylimino)-5-(2-methyl-benzooxazol-6-ylmethylene)-thiazolidin-4-one;
5-(2-Methyl-benzooxazol-6-ylmethylene)-2-(3-morpholin-4-yl-propylimino)-thiazolidin-4-one;
3-[5-(2-Methyl-benzooxazol-6-ylmethylene)-4-oxo-thiazolidin-2-ylideneamino]-benzenesulfonamide;
2-(4-Hydroxy-butylimino)-5-(2-methyl-benzooxazol-6-ylmethylene)-thiazolidin-4-one;
2-(trans-4-Hydroxy-cyclohexylimino)-5-(2-methyl-benzooxazol-6-ylmethylene)-thiazolidin-4-one;
5-(2-Methyl-benzooxazol-6-ylmethylene)-2-phenethylimino-thiazolidin-4-one;
4-{2-[5-(2-Methyl-benzooxazol-6-ylmethylene)-4-oxo-thiazolidin-2-ylideneamino]-ethyl}-benzenesulfonamide;
2-(2-Benzo[1,3]dioxol-5-yl-ethylimino)-5-(2-methyl-benzooxazol-6-ylmethylene)-thiazolidin-4-one;
2-(4-Chloro-phenylimino)-5-(2-methyl-benzooxazol-6-ylmethylene)-thiazolidin-4-one;
5-(2-Methyl-benzooxazol-6-ylmethylene)-2-(pyridin-3-ylimino)-thiazolidin-4-one;
3-[5-(2-Methyl-benzooxazol-6-ylmethylene)-4-oxo-thiazolidin-2-ylideneamino]-benzamide;
2-(2-Hydroxy-ethylimino)-5-(2-methyl-benzooxazol-6-ylmethylene)-thiazolidin-4-one;
2-(1-Hydroxymethyl-2-phenyl-ethylimino)-5-(2-methyl-benzooxazol-6-ylmethylene)-thiazolidin-4-one;
N-{6-[2-(2-Bromo-phenylimino)-4-oxo-thiazolidin-5-ylidenemethyl]-1H-benzoimidazol-2-yl}-2-dimethylamino-acetamide;
Methyl (5-{(Z)-[2-[(2-bromophenyl)amino]-4-oxo-1,3-thiazol-5(4H)-ylidene]methyl}-1H-benzimidazol-2-yl)carbamate;
(5Z)-2-[(2-chlorophenyl)amino]-5-{[1-(3,3-dimethylbutyl)-1H-benzimidazol-6-yl]methylidene}-1,3-thiazol-4(5H)-one;
(5Z)-2-[(2,6-dichlorophenyl)amino]-5-{[1-(3,3-dimethylbutyl)-1H-benzimidazol-6-yl]methylidene}-1,3-thiazol-4(5H)-one, trifluoroacetate salt;
(5Z)-5-{[1-(2-cyclopropylethyl)-1H-benzimidazol-6-yl]methylidene}-2-[(2,6-difluorophenyl)amino]-1,3-thiazol-4(5H)-one;
(5Z)-5-{[1-(2-cyclohexylethyl)-1H-benzimidazol-6-yl]methylidene}-2-[(2,6-dichlorophenyl)amino]-1,3-thiazol-4(5H)-one, trifluoroacetate salt;
(2Z,5Z)-2-[(2,6-dichlorophenyl)imino]-5-[(2-phenyl-1H-benzimidazol-6-yl)methylidene]-1,3-thiazolidin-4-one, piperidine salt;
(5Z)-2-[(2-chlorophenyl)amino]-5-{[1-(2-cyclopropylethyl)-1H-benzimidazol-6-yl]methylidene}-1,3-thiazol-4(5H)-one;
(5Z)-5-{[1-(2-cyclopropylethyl)-1H-benzimidazol-6-yl]methylidene}-2-[(2,6-dichlorophenyl)amino]-1,3-thiazol-4(5H)-one;
(2Z,5Z)-2-[(2,6-dichlorophenyl)imino]-5-{[2-(1-methylethyl)-1H-benzimidazol-6-yl]methylidene}-1,3-thiazolidin-4-one;
(2Z,5Z)-2-[(2,6-dichlorophenyl)imino]-5-{[2-(2-methylpropyl)-1H-benzimidazol-6-yl]methylidene}-1,3-thiazolidin-4-one, piperidine salt;
(2Z,5Z)-2-[(2,6-dichlorophenyl)imino]-5-{[2-(3-pyridinyl)-1H-benzimidazol-6-yl]methylidene}-1,3-thiazolidin-4 one;
(2Z,5Z)-2-[(2,6-dichlorophenyl)imino]-5-{[2-(hydroxymethyl)-1H-benzimidazol-5-yl]methylidene}-1,3-thiazolidin-4-one;
(2Z,5Z)-2-[(2,6-dichlorophenyl)imino]-5-{[2-(2-hydroxyethyl)-1H-benzimidazol-5-yl]methylidene}-1,3-thiazolidin-4-one;
(2Z,5Z)-2-[(2,6-dichlorophenyl)imino]-5-{[2-(2-pyridinyl)-1H-benzimidazol-6-yl]methylidene}-1,3-thiazolidin-4-one;
(5Z)-5-{[1-(2-cyclopentylethyl)-1H-benzimidazol-6-yl]methylidene}-2-[(2,6-dichlorophenyl)amino]-1,3-thiazol-4(5H)-one;
(5Z)-2-[(2-chlorophenyl)amino]-5-{[1-(2-cyclopentylethyl)-1H-benzimidazol-6-yl]methylidene}-1,3-thiazol-4(5H)-one;
(2Z,5Z)-2-[(2,6-dichlorophenyl)imino]-5-[(2-methyl-1H-benzimidazol-6-yl)methylidene]-1,3-thiazolidin-4-one;
(2Z,5Z)-2-[(2,6-dichlorophenyl)imino]-5-{[2-(4-pyridinyl)-1H-benzimidazol-5-yl]methylidene}-1,3-thiazolidin-4-one;
(2Z,5Z)-5-{[1-(2-cyclopropylethyl)-2-(3-pyridinyl)-1H-benzimidazol-6-yl]methylidene}-2-[(2,6-dichlorophenyl)imino]-1,3-thiazolidin-4-one;
(2Z,5Z)-2-[(2,6-dichlorophenyl)imino]-5-{[1-methyl-2-(3-pyridinyl)-1H-benzimidazol-5-yl]methylidene}-1,3-thiazolidin-4-one;
(2Z,5Z)-5-{[2-(aminomethyl)-1H-benzimidazol-5-yl]methylidene}-2-[(2,6-dichlorophenyl)imino]-1,3-thiazolidin-4-one;
(2Z,5Z)-5-(1H-benzimidazol-5-ylmethylidene)-2-[(2,6-dichlorophenyl)imino]-1,3-thiazolidin-4-one;
(2Z,5Z)-2-[(2,6-dichlorophenyl)imino]-5-{[2-(hydroxymethyl)-1-methyl-1H-benzimidazol-6-yl]methylidene}-1,3-thiazolidin-4-one;
(5Z)-2-[(2,6-dichlorophenyl)amino]-5-({1-[2-(3-pyridinyl)ethyl]-1H-benzimidazol-6-yl}methylidene)-1,3-thiazol-4(5H)-one;
(5Z)-5-{[1-(cyclopropylmethyl)-1H-benzimidazol-6-yl]methylidene}-2-[(2,6-dichlorophenyl)amino]-1,3-thiazol-4(5H)-one;
(5Z)-5-[(1-cyclopentyl-1H-benzimidazol-6-yl)methylidene]-2-[(2,6-dichlorophenyl)amino]-1,3-thiazol-4(5H)-one; and
(5Z)-5-(1,3-Benzoxazol-6-ylmethylidene)-2-[(2,6-dichlorophenyl)amino]-1,3-thiazol-4(5H)-one;
and/or a pharmaceutically acceptable salt, hydrate, solvate or pro-drug thereof.

14. A method of claim 1 wherein the compound of formula (I), and/or a pharmaceutically acceptable salt, hydrate, solvate or pro-drug thereof, is administered in a pharmaceutical composition.
